SEQUENCE LISTING (176 sequences)

SEQ ID NO: 1 - protein, 500 aa, Artificial Sequence
  source: /note=“Description of Artificial Sequence Synthetic polypeptide”
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 2 - protein, 507 aa, Artificial Sequence
  source: /note=“Description of Artificial Sequence Synthetic polypeptide”
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 3 - protein, 529 aa, Artificial Sequence
  source: /note=“Description of Artificial Sequence Synthetic polypeptide”
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 726 aa, Artificial Sequence
  source: /note=“Description of Artificial Sequence Synthetic polypeptide”
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 5 - protein, 517 aa, Artificial Sequence
  source: /note=“Description of Artificial Sequence Synthetic polypeptide”
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 6 - protein, 481 aa, Artificial Sequence
  source: /note=“Description of Artificial Sequence Synthetic polypeptide”
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 7 - protein, 358 aa, Artificial Sequence
  source: /note=“Description of Artificial Sequence Synthetic polypeptide”
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 8 - protein, 507 aa, Artificial Sequence
  source: /note=“Description of Artificial Sequence Synthetic polypeptide”
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 9 - protein, 534 aa, Artificial Sequence
  source: /note=“Description of Artificial Sequence Synthetic polypeptide”
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 10 - protein, 537 aa, Artificial Sequence
  source: /note=“Description of Artificial Sequence Synthetic polypeptide”
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 11 - protein, 529 aa, Artificial Sequence
  source: /note=“Description of Artificial Sequence Synthetic polypeptide”
MPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 12 - protein, 542 aa, Artificial Sequence
  source: /note=“Description of Artificial Sequence Synthetic polypeptide”
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 13 - protein, 564 aa, Artificial Sequence
  source: /note=“Description of Artificial Sequence Synthetic polypeptide”
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 14 - protein, 610 aa, Artificial Sequence
  source: /note=“Description of Artificial Sequence Synthetic polypeptide”
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 15 - protein, 369 aa, Artificial Sequence
  source: /note=“Description of Artificial Sequence Synthetic polypeptide”
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 16 - protein, 552 aa, Artificial Sequence
  source: /note=“Description of Artificial Sequence Synthetic polypeptide”
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 17 - protein, 534 aa, Artificial Sequence
  source: /note=“Description of Artificial Sequence Synthetic polypeptide”
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 18 - protein, 577 aa, Artificial Sequence
  source: /note=“Description of Artificial Sequence Synthetic polypeptide”
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 19 - protein, 613 aa, Artificial Sequence
  source: /note=“Description of Artificial Sequence Synthetic polypeptide”
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 20 - protein, 615 aa, Artificial Sequence
  source: /note=“Description of Artificial Sequence Synthetic polypeptide”
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 21 - protein, 775 aa, Artificial Sequence
  source: /note=“Description of Artificial Sequence Synthetic polypeptide”
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 22 - protein, 777 aa, Artificial Sequence
  source: /note=“Description of Artificial Sequence Synthetic polypeptide”
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 23 - protein, 610 aa, Artificial Sequence
  source: /note=“Description of Artificial Sequence Synthetic polypeptide”
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 24 - protein, 632 aa, Artificial Sequence
  source: /note=“Description of Artificial Sequence Synthetic polypeptide”
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 25 - protein, 625 aa, Artificial Sequence
  source: /note=“Description of Artificial Sequence Synthetic polypeptide”
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 26 - protein, 517 aa, Artificial Sequence
  source: /note=“Description of Artificial Sequence Synthetic polypeptide”
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 27 - protein, 410 aa, Artificial Sequence
  source: /note=“Description of Artificial Sequence Synthetic polypeptide”
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 28 - protein, 486 aa, Artificial Sequence
  source: /note=“Description of Artificial Sequence Synthetic polypeptide”
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 29 - protein, 602 aa, Artificial Sequence
  source: /note=“Description of Artificial Sequence Synthetic polypeptide”
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 30 - protein, 494 aa, Artificial Sequence
  source: /note=“Description of Artificial Sequence Synthetic polypeptide”
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 31 - protein, 526 aa, Artificial Sequence
  source: /note=“Description of Artificial Sequence Synthetic polypeptide”
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 32 - protein, 649 aa, Artificial Sequence
  source: /note=“Description of Artificial Sequence Synthetic polypeptide”
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 33 - protein, 414 aa, Artificial Sequence
  source: /note=“Description of Artificial Sequence Synthetic polypeptide”
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 34 - protein, 413 aa, Artificial Sequence
  source: /note=“Description of Artificial Sequence Synthetic polypeptide”
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 35 - protein, 449 aa, Artificial Sequence
  source: /note=“Description of Artificial Sequence Synthetic polypeptide”
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 36 - protein, 711 aa, Artificial Sequence
  source: /note=“Description of Artificial Sequence Synthetic polypeptide”
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 37 - protein, 574 aa, Artificial Sequence
  source: /note=“Description of Artificial Sequence Synthetic polypeptide”
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 38 - protein, 400 aa, Artificial Sequence
  source: /note=“Description of Artificial Sequence Synthetic polypeptide”
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 39 - protein, 743 aa, Artificial Sequence
  source: /note=“Description of Artificial Sequence Synthetic polypeptide”
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 40 - protein, 769 aa, Artificial Sequence
  source: /note=“Description of Artificial Sequence Synthetic polypeptide”
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 41 - protein, 666 aa, Artificial Sequence
  source: /note=“Description of Artificial Sequence Synthetic polypeptide”
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 42 - protein, 564 aa, Artificial Sequence
  source: /note=“Description of Artificial Sequence Synthetic polypeptide”
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 43 - protein, 565 aa, Artificial Sequence
  source: /note=“Description of Artificial Sequence Synthetic polypeptide”
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 44 - protein, 499 aa, Artificial Sequence
  source: /note=“Description of Artificial Sequence Synthetic polypeptide”
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 45 - protein, 560 aa, Artificial Sequence
  source: /note=“Description of Artificial Sequence Synthetic polypeptide”
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 46 - protein, 404 aa, Artificial Sequence
  source: /note=“Description of Artificial Sequence Synthetic polypeptide”
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 47 - protein, 392 aa, Artificial Sequence
  source: /note=“Description of Artificial Sequence Synthetic polypeptide”
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 48 - protein, 500 aa, Artificial Sequence
  source: /note=“Description of Artificial Sequence Synthetic polypeptide”
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 49 - protein, 507 aa, Artificial Sequence
  source: /note=“Description of Artificial Sequence Synthetic polypeptide”
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 50 - protein, 529 aa, Artificial Sequence
  source: /note=“Description of Artificial Sequence Synthetic polypeptide”
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 51 - protein, 517 aa, Artificial Sequence
  source: /note=“Description of Artificial Sequence Synthetic polypeptide”
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 52 - protein, 481 aa, Artificial Sequence
  source: /note=“Description of Artificial Sequence Synthetic polypeptide”
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 53 - protein, 534 aa, Artificial Sequence
  source: /note=“Description of Artificial Sequence Synthetic polypeptide”
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 54 - protein, 537 aa, Artificial Sequence
  source: /note=“Description of Artificial Sequence Synthetic polypeptide”
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 55 - protein, 541 aa, Artificial Sequence
  source: /note=“Description of Artificial Sequence Synthetic polypeptide”
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 56 - protein, 542 aa, Artificial Sequence
  source: /note=“Description of Artificial Sequence Synthetic polypeptide”
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 57 - protein, 564 aa, Artificial Sequence
  source: /note=“Description of Artificial Sequence Synthetic polypeptide”
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 58 - protein, 610 aa, Artificial Sequence
  source: /note=“Description of Artificial Sequence Synthetic polypeptide”
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 59 - protein, 552 aa, Artificial Sequence
  source: /note=“Description of Artificial Sequence Synthetic polypeptide”
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 60 - protein, 534 aa, Artificial Sequence
  source: /note=“Description of Artificial Sequence Synthetic polypeptide”
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 61 - protein, 577 aa, Artificial Sequence
  source: /note=“Description of Artificial Sequence Synthetic polypeptide”
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 62 - protein, 613 aa, Artificial Sequence
  source: /note=“Description of Artificial Sequence Synthetic polypeptide”
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 63 - protein, 615 aa, Artificial Sequence
  source: /note=“Description of Artificial Sequence Synthetic polypeptide”
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 64 - protein, 775 aa, Artificial Sequence
  source: /note=“Description of Artificial Sequence Synthetic polypeptide”
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 65 - protein, 777 aa, Artificial Sequence
  source: /note=“Description of Artificial Sequence Synthetic polypeptide”
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 66 - protein, 610 aa, Artificial Sequence
  source: /note=“Description of Artificial Sequence Synthetic polypeptide”
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 67 - protein, 632 aa, Artificial Sequence
  source: /note=“Description of Artificial Sequence Synthetic polypeptide”
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 68 - protein, 625 aa, Artificial Sequence
  source: /note=“Description of Artificial Sequence Synthetic polypeptide”
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 69 - protein, 517 aa, Artificial Sequence
  source: /note=“Description of Artificial Sequence Synthetic polypeptide”
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 70 - protein, 410 aa, Artificial Sequence
  source: /note=“Description of Artificial Sequence Synthetic polypeptide”
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 71 - protein, 602 aa, Artificial Sequence
  source: /note=“Description of Artificial Sequence Synthetic polypeptide”
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 72 - protein, 494 aa, Artificial Sequence
  source: /note=“Description of Artificial Sequence Synthetic polypeptide”
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 73 - protein, 649 aa, Artificial Sequence
  source: /note=“Description of Artificial Sequence Synthetic polypeptide”
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 74 - protein, 414 aa, Artificial Sequence
  source: /note=“Description of Artificial Sequence Synthetic polypeptide”
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 75 - protein, 413 aa, Artificial Sequence
  source: /note=“Description of Artificial Sequence Synthetic polypeptide”
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 76 - protein, 449 aa, Artificial Sequence
  source: /note=“Description of Artificial Sequence Synthetic polypeptide”
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 77 - protein, 711 aa, Artificial Sequence
  source: /note=“Description of Artificial Sequence Synthetic polypeptide”
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 78 - protein, 574 aa, Artificial Sequence
  source: /note=“Description of Artificial Sequence Synthetic polypeptide”
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 79 - protein, 743 aa, Artificial Sequence
  source: /note=“Description of Artificial Sequence Synthetic polypeptide”
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 80 - protein, 769 aa, Artificial Sequence
  source: /note=“Description of Artificial Sequence Synthetic polypeptide”
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 81 - protein, 564 aa, Artificial Sequence
  source: /note=“Description of Artificial Sequence Synthetic polypeptide”
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 82 - protein, 565 aa, Artificial Sequence
  source: /note=“Description of Artificial Sequence Synthetic polypeptide”
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 83 - protein, 499 aa, Artificial Sequence
  source: /note=“Description of Artificial Sequence Synthetic polypeptide”
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 84 - protein, 358 aa, Artificial Sequence
  source: /note=“Description of Artificial Sequence Synthetic polypeptide”
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 85 - protein, 369 aa, Artificial Sequence
  source: /note=“Description of Artificial Sequence Synthetic polypeptide”
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 86 - protein, 486 aa, Artificial Sequence
  source: /note=“Description of Artificial Sequence Synthetic polypeptide”
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 87 - protein, 400 aa, Artificial Sequence
  source: /note=“Description of Artificial Sequence Synthetic polypeptide”
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 88 - protein, 666 aa, Artificial Sequence
  source: /note=“Description of Artificial Sequence Synthetic polypeptide”
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 89 - protein, 560 aa, Artificial Sequence
  source: /note=“Description of Artificial Sequence Synthetic polypeptide”
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 90 - protein, 404 aa, Artificial Sequence
  source: /note=“Description of Artificial Sequence Synthetic polypeptide”
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 91 - protein, 392 aa, Artificial Sequence
  source: /note=“Description of Artificial Sequence Synthetic polypeptide”
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 92 - DNA, 1503 bp, Artificial Sequence
  source: /note=“Description of Artificial Sequence Synthetic polynucleotide”
atggaagtgcagaaaaccgtgatgaaaaccctgagcctgcggatcctgcggcctctgtactctcaagagatcgagaaagagatcaaagaggaaaaagagcgccggaagcaggctggcggaacaggcgaacttgatggcggcttctacaagaagctggaaaagaaacacagcgagatgttcagcttcgaccggctgaacctgctgctgaatcagctgcagcgggaaatcgccaaggtgtacaaccacgccatcagcgagctgtatatcgccacaatcgcccagggcaacaagagcaacaagcactacatcagcagcatcgtgtacaacagagcctacggctacttctacaacgcctatatcgccctgggcatctgcagcaaggtggaagccaacttcagaagcaacgagctgctgacccagcagagcgcactgcctacagccaagagcgacaacttccccatcgtgctgcacaagcagaaaggcgccgaaggcgaggatggcggattcagaatcagcaccgagggcagcgacctgatcttcgagatccccattccattctacgagtacaacggcgagaaccggaaagaaccctacaaatgggtcaagaaaggcgggcagaaaccagtgctgaagctgatcctgagcaccttccggcggcagagaaacaaaggctgggccaaagatgagggcaccgacgccgagatcagaaaagtgacagagggcaagtaccaggtgtcccagatcgagatcaaccggggcaagaaactgggcgagcaccagaagtggttcgccaatttcagcatcgagcagcccatctacgagcggaagcccaacagatctatcgtcggcggcctggacgtgggcattagatctccactcgtgtgcgccatcaacaacagcttctcccggtacagcgtggacagcaacgacgtgttcaagttcagcaaacaggtgttcgccttcagacggcggctgctgagcaagaacagcctgaagagaaaaggccacggcgctgcccacaagctggaacctatcaccgagatgaccgagaagaacgacaagttccggaagaaaatcatcgagcgctgggctaaagaagtgaccaacttcttcgtgaagaatcaagtgggcattgtgcagatcgaggacctgtccaccatgaaggacagagaggaccacttcttcaaccagtacctgagaggcttctggccctactaccagatgcagaccctgatcgagaacaagctgaaagaatacggcatcgaagtgaagcgcgtgcaggccaagtacaccagccagctgtgcagcaaccccaactgccggtactggaacaactacttcaacttcgagtatcggaaagtgaacaagttccccaagttcaagtgcgagaagtgcaacctggaaatcagcgccgactacaatgccgccagaaacctgagcacacccgacatcgagaagttcgtggccaaggccaccaagggcatcaacctgcctgagaagtga

SEQ ID NO: 93 - DNA, 1614 bp, Artificial Sequence
  source: /note=“Description of Artificial Sequence Synthetic polynucleotide”
atggaagagtccatcatcaccggcgtgaagttcaagctgcggatcgacaaagaaaccaccaagaagctgaacgagtacttcgacgagtacggcaaggccatcaacttcgccgtgaagatcatccagaaagagctggccgacgacagattcgccggaaaggccaagctggaccagaacaagaaccccatcctggacgagaatggcaagaagatctacgagttccccgacgagttctgcagctgcggcaagcaagtgaacaaatacgtgaacaacaagcccttctgccaagagtgctacaagatccggttcaccgagaacggcatccggaagagaatgtacagcgccaagggcagaaaggccgagcacaagatcaacatcctgaacagcaccaacaagatcagcaagacccacttcaactacgccatcagagaggccttcatcctggataagagcatcaagaagcagcggaagaagcgcaacgagcggctgagagagagcaagaagagactgcagcagttcatcgacatgcgcgacggcaagagagagatctgccctaccatcaagggccagaaggtggaccggttcattcaccccagctggatcaccaaggacaagaagctcgaggacttccggggctacaccctgagcatcatcaacagcaagatcaagattctggaccggaacatcaagcgggaagagaagtccctgaaagagaaggggcagatcatcttcaaggccaagagactgatgctggacaagtccatcagattcgtgggcgacagaaaggtgctgtttaccatctccaagacactgcccaaagagtacgagctggacctgcctagcaaagagaagcggctgaactggctgaaagaaaagatcgagatcatcaagaaccagaagccgaagtacgcctacctgctgcggaagaacatcgagagcgagaagaagcccaattacgagtactacctgcagtacaccctggaaatcaagcccgagctgaaggacttctacgacggcgccatcggcatcgacagaggcatcaatcacattgccgtgtgcaccttcatcagcaacgacggcaaagtgacccctcctaagttcttcagcagcggcgagatcctgagactgaagaacctgcagaaagaacgcgaccggttcctgctgagaaagcacaacaagaatcggaagaaaggcaacatgcgcgtgatcgagaacaagattaacctgatcctgcaccggtacagcaagcagatcgtggacatggccaaaaagctgaatgccagcatcgtgttcgaggaactgggcagaatcggcaagagccggaccaagatgaagaagtcccagcggtacaagctgagcctgttcatctttaagaaactgagcgacctggtggactacaagtctcggagagaaggcatcagagtgacctacgtgccaccagagtacaccagcaaagagtgctctcactgcggagagaaagtgaacacccagcggcctttcaacggcaactactccctgttcaagtgcaacaagtgcggcatccagctgaacagcgactacaacgccagcatcaatatcgccaagaagggcctgaagatccccaactccacctga

SEQ ID NO: 94 - DNA, 1593 bp, Artificial Sequence
  source: /note=“Description of Artificial Sequence Synthetic polynucleotide”
atgcccaagcaggatctggtcaccaccggcatcaagttcaagctggacgtggacaaagagacacggaagaaactggacgactacttcgacgagtacggcaaggccatcaacttcgccgtgaagatcatccagaagaacctgaaagaggaccgcttcgccggcaagattgccctgggcgaagataagaagcccctgctggacaaggacggcaagaagatctacaactaccccaacgagagctgctcctgcggcaatcaagtgcggagatacgtgaacgccaagcctttctgcgtggactgctacaagctgaagttcaccgagaacggcatccggaagcggatgtactctgccagaggaagaaaggccgacagcgacatcaacatcaagaacagcaccaacaagatcagcaagacccacttcaactacgccatcagagagggcttcatcctggacaagagcctgaagaagcagcggagcaagcggatcaagaagctgctggaactgaagcggaagctgcaagagttcatcgacatccggcagggccagatggtgctgtgccccaagatcaagaaccagagagtggacaagttcattcaccccagctggctgaagagagacaaaaagctggaagaattccggggctacagcctgagcgtggtggaaggcaagattaagatcttcaaccggaacatcctgcgcgaagaggactccctgagacagaggggccacgtgaactttaaggccaaccggatcatgctggataagagcgtgcggttcctggacggcggcaaagtgaatttcaacctgaacaagggcctgccgaaagagtacctgctggatctgcccaagaaagagaacaagctgtcctggctgaacgaaaagatcagcctgatcaagctgcagaagcctaagtacgcctacctgctgagaagagagggcagctttttcatccagtacaccatcgagaacgtgcccaagaccttcagcgattacctgggcgccatcggcatcgacagaggcatctctcacattgccgtgtgcaccttcgtgtccaagaacggcgtgaacaaggcccctgtgttcttcagctctggcgagatcctgaagctgaaaagcctgcagaaacagagggacctgttcctgcggggcaagcacaacaaaatccggaagaaaagcaacatgcggaacatcgacaacaagattaacctgatcctgcacaagtacagccgcaacatcgtgaacctggccaagagcgagaaggcctttatcgtgttcgagaagctcgagaagatcaaaaagtcccggttcaagatgagcaagtccctgcagtataagctgagccagttcaccttcaagaaactgagcgacctggtcgagtacaaggccaagatcgagggcatcaaggtggactacgtgccacctgagtacaccagcaaagagtgctctcactgcggcgagaaggtggacacccagaggcctttcaacggcaacagcagcctgttcaagtgtaacaagtgccgggtgcagctgaacgccgactacaacgccagcatcaatatcgccaagaagtccctgaacatcagcaacaactga

SEQ ID NO: 95 - DNA, 1605 bp, Artificial Sequence
  source: /note=“Description of Artificial Sequence Synthetic polynucleotide”
atggaaaagaacaacagcgagcagaccagcatcaccaccggcatcaagttcaagctgaagctggacaaagagacaaaagagaagctgaacaactacttcgacgagtacggcaaggccatcaacttcgccgtgcggatcatccagatgcagctgaacgacgatagactggccggcaagtacaagcgggacgagaagggaaagcctatcctgggcgaagatggcaagaagatcctggaaatccccaacgacttctgcagctgcggcaatcaagtgaaccactacgtgaacggcgtgtccttctgccaagagtgctacaagaagcggttcagcgagaacggcatccggaagagaatgtacagcgccaagggcagaaaggccgagcaggacatcaacatcaagaacagcaccaacaagatcagcaagacccacttcaactacgccatcagagaggccttcaacctggacaagagcatcaagaagcagagggaaaagcgcttcaagaaactgaaggacatgaagcggaagctgcaagagttcctcgagatccgcgacggcaagagagtgatctgccccaagatcgagaagcagaaggtggaacggtacattcaccccagctggatcaacaaagagaagaagctggaagaattccggggctacagcctgagcatcgtgaacagcaagatcaagagcttcgaccggaacatccagcgcgaggaaaagagcctgaaagagaagggccagatcaacttcaaggcccagcggctgatgctggataagagcgtgaagttcctcaaggacaacaaggtgtccttcaccatcagcaaagagctgcccaagaccttcgagctggacctgcctaagaaagagaaaaaactgaactggctgaacgagaagctcgagatcattaagaaccagaagccgaagtacgcctacctgctgcgcaaagagaacaacatcttcctgcagtacaccctggacagcatccccgagatccacagcgaatattctggcgccgtgggcatcgatagaggcgtgtcacatatcgccgtgtacaccttcctggataaggacggaaagaacgagcggccattcttcctgagcagcagcggcatcctgcggctgaagaacctgcagaaagagcgggacaagttcctgcggaagaagcacaacaaaatccggaaaaagggcaacatgcggaacatcgagcagaagatcaacctgatcctgcacgagtactccaagcagatcgtgaactttgccaaggacaagaacgccttcatcgtgttcgagctgctggaaaagcccaagaaaagccgcgagcggatgagcaagaaaatccagtacaagctgtcccagttcaccttcaaaaagctgagcgacctggtggactacaaggccaagcgcgagggcatcaaagtgatctacgtggaacccgcctacaccagcaaggactgttctcactgtggcgagagagtgaacacccagcggcctttcaacggcaacttcagcctgttcaagtgcaacaagtgcggcatcgtgctgaacagcgactacaacgccagcctgaatatcgcccggaagggcctgaacatcagcgccaattga

SEQ ID NO: 96 - DNA, 30 bp, Artificial Sequence
  source: /note=“Description of Artificial Sequence Synthetic oligonucleotide”
tattaaatactcgtattgctgttcgattat

SEQ ID NO: 97 - RNA, 59 nt, Artificial Sequence
  source: /note=“Description of Artificial Sequence Synthetic oligonucleotide”
guugcagaacccgaauagacgaaugaaggaaugcaacuauuaaauacucguauugcugu

SEQ ID NO: 98 - RNA, 135 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuuc

SEQ ID NO: 99 - RNA, 180 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuucgguucaaaguucugcacaaaacaggugaguccuuauaaaccggug

SEQ ID NO: 100 - RNA, 59 nt, Artificial Sequence
  source: /note=“Description of Artificial Sequence Synthetic oligonucleotide”
cuuucauacucagaacaaagggauuaaggaaugcaacuauuaaauacucguauugcugu

SEQ ID NO: 101 - RNA, 112 nt, Artificial Sequence
  source: /note=“Description of Artificial Sequence Synthetic polynucleotide”
gaacagaccaaucuuuaauuccguucugauuuaaaaaaucagaaucucuuuauaaauaguauuacaaaaaguguacauuccaaaauccgaaagcagaauugaccuuuuuaag

SEQ ID NO: 102 - RNA, 59 nt, Artificial Sequence
  source: /note=“Description of Artificial Sequence Synthetic oligonucleotide”
auuucauacucagaacaaagggauuaaggaaugcaacuauuaaauacucguauugcugu

SEQ ID NO: 103 - RNA, 217 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 104 - RNA, 59 nt, Artificial Sequence
  source: /note=“Description of Artificial Sequence Synthetic oligonucleotide”
guugcgcgaauagaauaaaggaauuaaggaaugcaacuauuaaauacucguauugcugu

SEQ ID NO: 105 - RNA, 217 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 106 - RNA, 86 nt, Artificial Sequence
  source: /note=“Description of Artificial Sequence Synthetic oligonucleotide”
cgauuccucccuacaguaguuagguauagccgaaagguagagacuaaaucuguaguuggagugggccgcuugcaucggccuaaagu

SEQ ID NO: 107 - RNA, 50 nt, Artificial Sequence
  source: /note=“Description of Artificial Sequence Synthetic oligonucleotide”
cgauuccucccuacaguaguuagguauagccgaaagguagagacuaaauc

SEQ ID NO: 108 - RNA, 86 nt, Artificial Sequence
  source: /note=“Description of Artificial Sequence Synthetic oligonucleotide”
guuggagugggccgcuugcaucggccuaaaguugagaagugucagacucugauaacccucaacgacgauauucuuuauuucgguuc

SEQ ID NO: 109 - RNA, 97 nt, Artificial Sequence
  source: /note=“Description of Artificial Sequence Synthetic oligonucleotide”
cgauuccucccuacaguaguuagguauagccgaaagguagagacuaaaucuguaguuggagugggccgcuugcaucggccuaaaguugagaaguguc

SEQ ID NO: 110 - RNA, 140 nt, Artificial Sequence
  source: /note=“Description of Artificial Sequence Synthetic polynucleotide”
caucggccuaaaguugagaagugucagacucugauaacccucaacgacgauauucuuuauuucgguucaaaguucugcacaaaacaggugaguccuuauaaaccggugugcagaacgccggcucaccuuuuuccuucauc

SEQ ID NO: 111 - RNA, 115 nt, Artificial Sequence
  source: /note=“Description of Artificial Sequence Synthetic polynucleotide”
uagguauagccgaaagguagagacuaaaucuguaguuggagugggccgcuugcaucggccuaaaguugagaagugucagacucugauaacccucaacgacgauauucuuuauuuc

SEQ ID NO: 112 - RNA, 125 nt, Artificial Sequence
  source: /note=“Description of Artificial Sequence Synthetic polynucleotide”
cuacaguaguuagguauagccgaaagguagagacuaaaucuguaguuggagugggccgcuugcaucggccuaaaguugagaagugucagacucugauaacccucaacgacgauauucuuuauuuc

SEQ ID NO: 113 - RNA, 129 nt, Artificial Sequence
  source: /note=“Description of Artificial Sequence Synthetic polynucleotide”
cucccuacaguaguuagguauagccgaaagguagagacuaaaucuguaguuggagugggccgcuugcaucggccuaaaguugagaagugucagacucugauaacccucaacgacgauauucuuuauuuc

SEQ ID NO: 114 - RNA, 140 nt, Artificial Sequence
  source: /note=“Description of Artificial Sequence Synthetic polynucleotide”
gcacacgauuccucccuacaguaguuagguauagccgaaagguagagacuaaaucuguaguuggagugggccgcuugcaucggccuaaaguugagaagugucagacucugauaacccucaacgacgauauucuuuauuuc

SEQ ID NO: 115 - RNA, 145 nt, Artificial Sequence
  source: /note=“Description of Artificial Sequence Synthetic polynucleotide”
uugaugcacacgauuccucccuacaguaguuagguauagccgaaagguagagacuaaaucuguaguuggagugggccgcuugcaucggccuaaaguugagaagugucagacucugauaacccucaacgacgauauucuuuauuuc

SEQ ID NO: 116 - RNA, 130 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuu

SEQ ID NO: 117 - RNA, 140 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuucgguuc

SEQ ID NO: 118 - RNA, 155 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuucgguucaaaguucugcacaaa

SEQ ID NO: 119 - RNA, 81 nt, Artificial Sequence
  source: /note=“Description of Artificial Sequence Synthetic oligonucleotide”
guuggagugggccgcuugcaucggccuaaaguugagaagugucagacucugauaacccucaacgacgauauucuuuauuuc

SEQ ID NO: 120 - RNA, 170 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuucgguucaaaguucugcacaaaacaggugaguccuua

SEQ ID NO: 121 - RNA, 189 nt, Artificial Sequence
  source: /note=“Description of Artificial Sequence Synthetic polynucleotide”
cgauuccucccuacaguaguuagguauagccgaaagguagagacuaaaucuguaguuggagugggccgcuugcaucggccuaaaguugagaagugucagacucugauaacccucaacgacgauauucuuuauuucgguucaaaguucugcacaaaacaggugaguccuuauaaaccggugugcagaacg

SEQ ID NO: 122 - RNA, 170 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaa

SEQ ID NO: 123 - RNA, 151 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaac

SEQ ID NO: 124 - RNA, 125 nt, Artificial Sequence
  source: /note=“Description of Artificial Sequence Synthetic polynucleotide”
auuguaauauuaugguguugacuuaguuuagauuuaaacaaucuucgauggcuauaugcggaagguuuggcgucguuguaacgcaauagggggcgaccccgaaaagguuugauaucauaucaaac

SEQ ID NO: 125 - RNA, 227 nt, Artificial Sequence
  source: /note=“Description of Artificial Sequence Synthetic polynucleotide”
agcucuuuuguugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 126 - RNA, 237 nt, Artificial Sequence
  source: /note=“Description of Artificial Sequence Synthetic polynucleotide”
uccaauaacuagcucuuuuguugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 127 - RNA, 207 nt, Artificial Sequence
  source: /note=“Description of Artificial Sequence Synthetic polynucleotide”
caagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 128 - RNA, 197 nt, Artificial Sequence
  source: /note=“Description of Artificial Sequence Synthetic polynucleotide”
aaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 129 - RNA, 187 nt, Artificial Sequence
  source: /note=“Description of Artificial Sequence Synthetic polynucleotide”
uaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacu

SEQ ID NO: 130 - RNA, 227 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacuaaacgucaga

SEQ ID NO: 131 - RNA, 237 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacuaaacgucagaacaucuuuau

SEQ ID NO: 132 - RNA, 247 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacuaaacgucagaacaucuuuauaaaucaauuu

SEQ ID NO: 133 - RNA, 257 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccuuuaucugacuaaacgucagaacaucuuuauaaaucaauuuacaaaaauga

SEQ ID NO: 134 - RNA, 207 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauucuuaauaccu

SEQ ID NO: 135 - RNA, 197 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaaaugaacagaccaauu

SEQ ID NO: 136 - RNA, 182 nt, Artificial Sequence
  source: /note=“Description of Artificial Sequence Synthetic polynucleotide”
uugguuaagccaagauauggaaugccauuguaauauuaugguguugacuuaguuuagauuuaaacaaucuucgauggcuauaugcggaagguuuggcgucguuguaacgcaauagggggcgaccccgaaaagguuugauaucauaucaaacuuaguuuuguuuaaguuaaggcuuauucaaa

SEQ ID NO: 137 - RNA, 150 nt, Artificial Sequence
  source: /note=“Description of Artificial Sequence Synthetic polynucleotide”
auuuuauuugaguuucggcuugaguaaaaugaacagaccaauuuuuaauuucguucauaucaucgcaacuaagaauuccuuuaaaaaggaaaugcagaaaauggacauuccaaaauucaaaacaaaauucgguuuuuuaagaccagaaag

SEQ ID NO: 138 - RNA, 170 nt, Artificial Sequence
  source: /note=“Description of Artificial Sequence Synthetic polynucleotide”
acugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 139 - RNA, 207 nt, Artificial Sequence
  source: /note=“Description of Artificial Sequence Synthetic polynucleotide”
gaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 140 - RNA, 197 nt, Artificial Sequence
  source: /note=“Description of Artificial Sequence Synthetic polynucleotide”
cuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 141 - RNA, 187 nt, Artificial Sequence
  source: /note=“Description of Artificial Sequence Synthetic polynucleotide”
auuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 142 - RNA, 177 nt, Artificial Sequence
  source: /note=“Description of Artificial Sequence Synthetic polynucleotide”
aaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 143 - RNA, 237 nt, Artificial Sequence
  source: /note=“Description of Artificial Sequence Synthetic polynucleotide”
ugaacaucagcgccaauugaaguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacua

SEQ ID NO: 144 - RNA, 227 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacuaagaauuccuu

SEQ ID NO: 145 - RNA, 237 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacuaagaauuccuuuaaaaaggaa

SEQ ID NO: 146 - RNA, 247 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacuaagaauuccuuuaaaaaggaaaugcagaaaa

SEQ ID NO: 147 - RNA, 296 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauaucaucgcaacuaagaauuccuuuaaaaaggaaaugcagaaaauggacauuccaaaauucaaaacaaaauucgguuuuuuaagaccagaaag

SEQ ID NO: 148 - RNA, 207 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuucguucauauc

SEQ ID NO: 149 - RNA, 197 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaauuuuuaauuu

SEQ ID NO: 150 - RNA, 187 nt, Artificial Sequence
  source: /note=“Description of Artificial Sequence Synthetic polynucleotide”
aguguaaguugaagugugagcuuauggauuauuauuuacaaaauaauacugacuuacuaagauaucuugaggguauacccaaaaagauuggcguuguugcaacgcaauaagauguaaaucugaaaagguuugaaaucauauaaguaauuuuauuugaguuucggcuugaguaaaaugaacagaccaa

SEQ ID NO: 151 - RNA, 206 nt, Artificial Sequence
  source: /note=“Description of Artificial Sequence Synthetic polynucleotide”
augcggaagauuuggcguuguuguaacgcaauaagggguaacccugaaaagguuugaaaucauauaaaccuaguuuuauuugaguuuaggcucagauaaaaugaacagaccaaucuuuaauuccguucugauuuaaaaaaucagaaucucuuuauaaauaguauuacaaaaaguguacauuccaaaauccgaaagcagaauugacc

SEQ ID NO: 152 - DNA, 2195 bp, Artificial Sequence
  source: /note=“Description of Artificial Sequence Synthetic polynucleotide”
gtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgccatggacatgtttatattaaatactcgtattgctgttcgattatgaccgaattccctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtc

SEQ ID NO: 153 - RNA, 5 nt, Artificial Sequence
  source: /note=“Description of Artificial Sequence Synthetic oligonucleotide”
uuuuu

SEQ ID NO: 154 - RNA, 8 nt, Artificial Sequence
  source: /note=“Description of Artificial Sequence Synthetic oligonucleotide”
uuuuuuuu

SEQ ID NO: 155 - RNA, 10 nt, Artificial Sequence
  source: /note=“Description of Artificial Sequence Synthetic oligonucleotide”
uuuuuuuuuu

SEQ ID NO: 156 - DNA, 10 bp, Artificial Sequence
  source: /note=“Description of Artificial Sequence Synthetic oligonucleotide”
  source: /note=“Description of Combined DNA/RNA Molecule Synthetic oligonucleotide”
ttttuutttt

SEQ ID NO: 157 - DNA, 6 bp, Artificial Sequence
  source: /note=“Description of Artificial Sequence Synthetic oligonucleotide”
  source: /note=“Description of Combined DNA/RNA Molecule Synthetic oligonucleotide”
ttuutt

SEQ ID NO: 158 - DNA, 6 bp, Artificial Sequence
  source: /note=“Description of Artificial Sequence Synthetic oligonucleotide”
  source: /note=“Description of Combined DNA/RNA Molecule Synthetic oligonucleotide”
taaugc

SEQ ID NO: 159 - DNA, 6 bp, Artificial Sequence
  source: /note=“Description of Artificial Sequence Synthetic oligonucleotide”
  source: /note=“Description of Combined DNA/RNA Molecule Synthetic oligonucleotide”
tauggc

SEQ ID NO: 160 - RNA, 5 nt, Artificial Sequence
  source: /note=“Description of Artificial Sequence Synthetic oligonucleotide”
uuuuu

SEQ ID NO: 161 - DNA, 8 bp, Artificial Sequence
  source: /note=“Description of Artificial Sequence Synthetic oligonucleotide”
ttattatt

SEQ ID NO: 162 - DNA, 6 bp, Artificial Sequence
  source: /note=“Description of Artificial Sequence Synthetic oligonucleotide”
tttttt

SEQ ID NO: 163 - DNA, 8 bp, Artificial Sequence
  source: /note=“Description of Artificial Sequence Synthetic oligonucleotide”
tttttttt

SEQ ID NO: 164 - DNA, 10 bp, Artificial Sequence
  source: /note=“Description of Artificial Sequence Synthetic oligonucleotide”
tttttttttt

SEQ ID NO: 165 - DNA, 12 bp, Artificial Sequence
  source: /note=“Description of Artificial Sequence Synthetic oligonucleotide”
tttttttttttt

SEQ ID NO: 166 - DNA, 14 bp, Artificial Sequence
  source: /note=“Description of Artificial Sequence Synthetic oligonucleotide”
tttttttttttttt

SEQ ID NO: 167 - DNA, 6 bp, Artificial Sequence
  source: /note=“Description of Artificial Sequence Synthetic oligonucleotide”
aaaaaa

SEQ ID NO: 168 - DNA, 6 bp, Artificial Sequence
  source: /note=“Description of Artificial Sequence Synthetic oligonucleotide”
cccccc

SEQ ID NO: 169 - DNA, 6 bp, Artificial Sequence
  source: /note=“Description of Artificial Sequence Synthetic oligonucleotide”
gggggg

SEQ ID NO: 170 - protein, 726 aa, Artificial Sequence
  source: /note=“Description of Artificial Sequence Synthetic polypeptide”
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 171 - DNA, 12 bp, Artificial Sequence
  source: /note=“Description of Artificial Sequence Synthetic oligonucleotide”
aaaaaaaaaaaa

SEQ ID NO: 172 - DNA, 12 bp, Artificial Sequence
  source: /note=“Description of Artificial Sequence Synthetic oligonucleotide”
cccccccccccc

SEQ ID NO: 173 - DNA, 20 bp, Artificial Sequence
  source: /note=“Description of Artificial Sequence Synthetic oligonucleotide”
  misc_feature (1)..(20): /note=“This sequence may encompass 5-20 nucleotides”
aaaaaaaaaaaaaaaaaaaa

SEQ ID NO: 174 - DNA, 20 bp, Artificial Sequence
  source: /note=“Description of Artificial Sequence Synthetic oligonucleotide”
  misc_feature (1)..(20): /note=“This sequence may encompass 5-20 nucleotides”
tttttttttttttttttttt

SEQ ID NO: 175 - DNA, 20 bp, Artificial Sequence
  source: /note=“Description of Artificial Sequence Synthetic oligonucleotide”
  misc_feature (1)..(20): /note=“This sequence may encompass 5-20 nucleotides”
cccccccccccccccccccc

SEQ ID NO: 176 - DNA, 20 bp, Artificial Sequence
  source: /note=“Description of Artificial Sequence Synthetic oligonucleotide”
  misc_feature (1)..(20): /note=“This sequence may encompass 5-20 nucleotides”
gggggggggggggggggggg

(19) United States
(12) Patent Application Publication
(10) Pub. No.: US 2022/0049241 A1
(43) Pub. Date: Feb. 17, 2022

(54) PROGRAMMABLE NUCLEASES AND METHODS OF USE

(71) Applicant: MAMMOTH BIOSCIENCES, INC., Brisbane, CA (US)

(72) Inventors: Lucas Benjamin HARRINGTON, San Francisco, CA (US); Janice Sha CHEN, San Francisco, CA (US); Isaac Paterson WITTE, Walnut Creek, CA (US)

(21) Appl. No.: 17/516,027

(22) Filed: Nov. 1, 2021

Related U.S. Application Data
(63) Continuation of application No. PCT/US2020/031030, filed on May 1, 2020.
(60) Provisional application No. 62/841,770, filed on May 1, 2019.

(51) Int. Cl. C12N 15/10; A61K 31/7088; C12N 9/22

(52) U.S. Cl. CPC C12N 15/102; C12N 2310/20; C12N 9/22; A61K 31/7088

(57) ABSTRACT

Provided herein are programmable nucleases and methods of genome editing and detection of nucleic acids with said programmable nuclease. In some embodiments, the programmable nuclease is a programmable nickase.

### CROSS REFERENCE

This application is a continuation application of International Application No. PCT/US2020/031030, filed May 1, 2020 which claims priority to U.S. Provisional Patent Application No. 62/841,770, filed May 1, 2019 which is incorporated herein by reference in its entirety.

### SEQUENCE LISTING

This application incorporates by reference in its entirety the Computer Readable Form (“CRF”) of a Sequence Listing in ASCII text format submitted via EFS-Web. The Sequence Listing text file submitted via EFS-Web is entitled “53694-728_301_SL.txt,” was created on Nov. 1, 2021 and is 491,991 bytes in size.

### BACKGROUND

Certain programmable nucleases can be used for genome editing of nucleic acid sequences or detection of nucleic acid sequences. There is a need for high efficiency, programmable nickases that are capable of working under various sample conditions, and can be used for both genome editing and diagnostics.

### SUMMARY

In various aspects, the present disclosure provide a method of introducing a break in a target nucleic acid, the method comprising introducing the break by contacting the target nucleic acid with: (a) a first guide nucleic acid comprising a first region that binds to a first programmable nickase having a length of no more than 900 amino acids; and (b) a second guide nucleic acid comprising a first region that binds to a second programmable nickase having a length of no more than 900 amino acids, wherein the first guide nucleic acid comprises a second region that binds to the target nucleic acid and wherein the second guide nucleic acid comprises a second region that binds to the target nucleic acid and wherein the second region of the first guide nucleic acid and the second region of the second guide nucleic acid bind opposing strands of the target nucleic acid. In some aspects, the first programmable nickase and the second programmable nickase have a length of from 350 to 900 amino acids. In some aspects, the first programmable nickase and the second programmable nickase have a length of from 480 to 550 amino acids.

In some aspects, the first programmable nickase and second programmable nickase are a Type V CRISPR/Cas enzyme. In some aspects, the Type V CRISPR/Cas enzyme comprises three partial RuvC domains. In some aspects, the three partial RuvC domains are RuvC-I, RuvC-II, and RuvC-III subdomains. In some aspects, the first programmable nickase and the second programmable nickase are a Cas14 protein. In some aspects, the Cas14 protein is a Cas14a protein, a Cas14b protein, a Cas14c protein, a Cas14d protein, or a Cas14e protein. In some aspects, the Cas14 protein is a Cas14a protein. In some aspects, the Cas14 proteins is a Cas14b protein. In some aspects, the Cas14 protein is a Cas14e protein.

In some aspects, the first programmable nickase, the second programmable nickase, or both have at least 80%, at least 85%, at least 90%, at least 92%, at least 95%, at least 97%, or at least 99% sequence identity with any one of SEQ ID NO: 1-SEQ ID NO: 91 or SEQ ID NO: 170. In some aspects, the first programmable nickase, the second programmable nickase, or both are any one of SEQ ID NO: 1-SEQ ID NO: 91 or SEQ ID NO: 170.

In some aspects, the first programmable nickase, the second programmable nickase, or both have at least 80%, at least 85%, at least 90%, at least 92%, at least 95%, at least 97%, or at least 99% sequence identity with SEQ ID NO: 1. In some aspects, the first programmable nickase, the second programmable nickase, or both are SEQ ID NO: 1.

In some aspects, the first programmable nickase, the second programmable nickase, or both have at least 80%, at least 85%, at least 90%, at least 92%, at least 95%, at least 97%, or at least 99% sequence identity with SEQ ID NO: 10. In some aspects, the first programmable nickase, the second programmable nickase, or both are SEQ ID NO: 10.

In some aspects, the first programmable nickase, the second programmable nickase, or both have at least 80%, at least 85%, at least 90%, at least 92%, at least 95%, at least 97%, or at least 99% sequence identity with SEQ ID NO: 11. In some aspects, the first programmable nickase, the second programmable nickase, or both are SEQ ID NO: 11.

In some aspects, the first programmable nickase, the second programmable nickase, or both have at least 80%, at least 85%, at least 90%, at least 92%, at least 95%, at least 97%, or at least 99% sequence identity with SEQ ID NO: 17. In some aspects, the first programmable nickase, the second programmable nickase, or both are SEQ ID NO: 17.

In some aspects, the first programmable nickase, the second programmable nickase, or both have at least 80%, at least 85%, at least 90%, at least 92%, at least 95%, at least 97%, or at least 99% sequence identity with SEQ ID NO: 33. In some aspects, the first programmable nickase, the second programmable nickase, or both are SEQ ID NO: 33.

In some aspects, the first guide nucleic acid is a first guide RNA. In some aspects, the second guide nucleic acid is a second guide RNA. In some aspects, the first region is a repeat sequence and wherein the second region is a spacer sequence. In some aspects, the first guide nucleic acid and the second guide nucleic acid comprise a crRNA and a tracrRNA. In some aspects, the first guide nucleic acid and the second guide nucleic acid comprise a crRNA and a trancRNA. In some aspects, the crRNA comprises the repeat sequence and the spacer sequence. In some aspects, the repeat sequence hybridizes to a segment of the tracrRNA.

In some aspects, the tracrRNA has at least 80%, at least 85%, at least 90%, at least 92%, at least 95%, at least 97%, or at least 99% sequence identity with any one of SEQ ID NO: 98-SEQ ID NO: 99, SEQ ID NO: 101, SEQ ID NO: 103, SEQ ID NO: 105-SEQ ID NO: 151. In some aspects, the tracrRNA is any one of SEQ ID NO: 98-SEQ ID NO: 99, SEQ ID NO: 101, SEQ ID NO: 103, SEQ ID NO: 105-SEQ ID NO: 151.

In some aspects, the tracrRNA has at least 80%, at least 85%, at least 90%, at least 92%, at least 95%, at least 97%, or at least 99% sequence identity with SEQ ID NO: 99 In some aspects, the tracrRNA is SEQ ID NO: 99. In some aspects, the tracrRNA has at least 80%, at least 85%, at least 90%, at least 92%, at least 95%, at least 97%, or at least 99% sequence identity with SEQ ID NO: 101. In some aspects, the tracrRNA is SEQ ID NO: 101. In some aspects, the tracrRNA has at least 80%, at least 85%, at least 90%, at least 92%, at least 95%, at least 97%, or at least 99% sequence identity with SEQ ID NO: 103. In some aspects, the tracrRNA is SEQ ID NO: 103. In some aspects, the tracrRNA has at least 80%, at least 85%, at least 90%, at least 92%, at least 95%, at least 97%, or at least 99% sequence identity with SEQ ID NO: 119. In some aspects, the tracrRNA is SEQ ID NO: 119.

In some aspects, the first programmable nickase and the second programmable nickase exhibit 2-fold greater nicking activity as compared to double stranded cleavage activity. In some aspects, the first programmable nickase and the second programmable nickase nick the target nucleic acid at two different sites. In some aspects, the target nucleic acid comprises double stranded DNA. In some aspects, the two different sites are on opposing strands of the double stranded DNA. In some aspects, the target nucleic acid comprises a mutated sequence or a sequence is associated with a disease. In some aspects, the disease is cancer.

In some aspects, the method comprises administering the first programmable nickase and the second programmable nickase to a subject in need thereof. In some aspects, the mutated sequence is removed after the first programmable nickase and the second programmable nickase nick the target nucleic acid. In some aspects, the first programmable nickase and the second programmable nickase are the same. In some aspects, the first programmable nickase and the second programmable nickase are different. In various aspects, the present disclosure provides a method of introducing a strand break in a target nucleic acid, the method comprising introducing the strand break by contacting the target nucleic acid with: (a) a first guide RNA comprising a first region that binds to a first programmable nickase; and (b) a second guide RNA comprising a first region that binds to a second programmable nickase, wherein the first guide RNA comprises a second region that binds to the target nucleic acid and wherein the second guide RNA comprises a second region that binds to the target nucleic acid and wherein the second region of the first guide RNA and the second region of the second guide RNA bind opposing strands of the target nucleic acid.

In some aspects, the first programmable nickase and the second programmable nickase nick the target nucleic acid at two different sites. In some aspects, the target nucleic acid comprises double stranded DNA. In some aspects, the two different sites are on opposing strands of the double stranded DNA. In some aspects, the target nucleic acid comprises a mutated sequence or a sequence is associated with a disease. In further aspects, the disease is cancer.

In some aspects, the method comprises administering the first programmable nickase and the second programmable nickase to a subject in need thereof. In some aspects, the mutated sequence is removed after the first programmable nuclease and the second programmable nuclease nick the target nucleic acid. In some aspects, the first programmable nickase and the second programmable nickase comprise a Cas14 protein.

In various aspects, the present disclosure provides a method of detecting a target nucleic acid in a sample, the method comprising contacting the sample with (a) a programmable nickase; (b) a guide RNA comprising a first region that binds to the programmable nickase and a second region that binds to the target nucleic acid; and (c) a labeled, single stranded DNA reporter that does not bind the guide RNA; cleaving the labeled single stranded DNA reporter to release a detectable label; and detecting the target nucleic acid by measuring a signal from the detectable label.

In some aspects, the target nucleic acid is single stranded DNA. In some aspects, the programmable nickase comprises a Cas 14 protein. In some aspects, the target nucleic acid is in a sample. In some aspects, the sample comprises a phosphate buffer, a Tris buffer, or a HEPES buffer. In further aspects, the sample comprises a pH of 7 to 9. In still further aspects, the sample comprises a pH of 7.5 to 8. In some aspects, the sample comprises a salt concentration of 25 nM to 200 mM.

In some aspects, the single stranded DNA reporter comprises an ssDNA-fluorescence quenching DNA reporter. In further aspects, the ssDNA-fluorescence quenching DNA reporter is a universal ssDNA-fluorescence quenching DNA reporter. In some aspects, the programmable nickase exhibits PAM-independent nicking and cleaving. In some aspects, the Cas14 protein comprises a Cas14e protein. In some aspects, the Cas14 protein comprises from 400 to 800 amino acid residues.

In various aspects, the present disclosure provides a composition comprising a programmable nickase and a guide RNA comprising a first region that binds the programmable nickase and a second region that binds a target nucleic acid.

In some aspects, the target nucleic acid comprises single stranded DNA or double stranded DNA. In some aspects, the programmable nickase exhibits PAM-independent nicking and cleaving. In some aspects, the programmable nickase nicks a single strand of the double stranded DNA. In some aspects, the programmable nickase cleaves single stranded DNA. In some aspects, the programmable nickase comprises a Cas14 protein. In further aspects, the Cas14 protein comprises a Cas14e protein. In still further aspects, the Cas14 protein comprises from 400 to 800 amino acid residues.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a gel illustrating nicking of dsDNA by a programmable nickase. Four programmable nickases, which here are four Cas14e proteins, were independently added to the first four lanes along with a guide RNA (TRACR2), which forms a complex with a programmable nickase. When the guide RNA is complexed with the programmable nickase and when this complex binds to its target nucleic acid, the nickase activity of the programmable nickase is activated. This is shown in the first four lanes of the gel by the resulting two bands, in which the upper band is the nicked target dsDNA. The fifth lane is a control lane comprising a programmable nickase, but no guide RNA, in which the target dsDNA remains intact. The sixth lane shows cleavage of dsDNA by a restriction enzyme, EcoRI, which generates a double strand break. The seventh lane shows untreated target dsDNA (e.g., no programmable nickase, guide RNA, or restriction enzyme).

FIG. 2 shows the effect of salt, buffer, and temperature on a ssDNA DETECTR reaction using Cas14e. At the top left is a bar graph showing various buffer conditions and pH levels on the x-axis and the background subtracted fluorescence on the y-axis. Fluorescence indicates cleavage of a reporter. Greater fluorescence indicates more activity. At the top middle and top right are graphs showing temperatures on the x-axis (“ON” indicates the target ssDNA that can hybridize to the guide RNA was added; “OFF” indicates off-target ssDNA that does not hybridize to the guide RNA was added) versus raw fluorescence on the y-axis. The “OFF” fluorescence is used to determine background fluorescence. The bottom three line graphs show fluorescence over time in various salt conditions (25 nM NaCl, 100 nM NaCl, and 200 mM NaCl from left to right). Fluorescence indicates cleavage of a reporter. Greater fluorescence indicates more activity. The higher line, with increasing fluorescence over time, shows cleavage of reporters by Cas14e proteins complexed with guide RNAs in the presence of target ssDNA. The lower line, with flat fluorescence over time, shows Cas14e proteins complexed with guide RNAs in the presence of off-target ssDNA.

FIG. 3 shows three graphs, which from left to right assess cleavage of homopolymer fluorescence-quenching (FQ) reporters. Fluorescence indicates cleavage of a reporter. Greater fluorescence indicates higher cleavage activity. The left most graph uses a T12 (12 thymine residues) ssDNA-FQ reporter, the middle graph uses an A12 (12 adenine residues) ssDNA-FQ reporter, and the right most graph uses a C12 (12 cytosine residues) ssDNA-FQ reporter. In each graph, the top lines show Cas14e proteins complexed with guide RNAs in the presence of target ssDNA and the bottom lines show Cas14e proteins complexed with guide RNAs in the presence of off-target ssDNA.

FIG. 4 shows a graph of fluorescence over time for three DETECTR reactions using Cas14e proteins coupled to a guide RNA to detect target dsDNA. Fluorescence indicates cleavage of a reporter. Greater fluorescence indicates more activity. The top most line shows cleavage of reporters in the presence of a target dsDNA having a wild type (wt) PAM. The line immediately below the top most line shows cleavage of reporters in the presence of a target dsDNA having a mutant (mut) PAM. The lowest line shows cleavage of reporters in the presence of 500 nM of off-target ssDNA. The results showed that Cas14e is insensitive to PAM restrictions.

FIG. 5 shows the results of cis-cleavage activity assays for four programmable nickases separately complexed with four distinct guide nucleic acids. The programmable nickases were incubated for 60 minutes with plasmid DNA targeted by the guide nucleic acids. The graph shows the percentage of plasmids that developed nicks (single-stranded breaks) or double-stranded breaks during the 60 minute incubation, as measured by gel-electrophoresis.

FIG. 6 shows the results of cis-cleavage activity assays for three distinct programmable nickases complexed with 18, 16, or 15 separate guide nucleic acids. The programmable nickases were incubated for 10 minutes with plasmid DNA targeted by the guide nucleic acids. The graphs show the percentage of plasmids exhibited nicks (single-stranded breaks; “nicked”) or double-stranded breaks (“cleaved”) for each programmable nickase-guide nucleic acid pair.

FIG. 6A shows the results for assays with Cas14a.3.

FIG. 6B shows the results for assays with Cas14b.4.

FIG. 6C shows the results for assays with Cas14b.10.

### DETAILED DESCRIPTION

The present disclosure provides compositions of programmable nucleases. In some embodiments, the programmable nuclease is a programmable DNA nuclease. These programmable nucleases can be complexed with a guide RNA that can bind to a target DNA. In certain embodiments, when the programmable nuclease is complexed with the guide RNA and the target DNA hybridizes to the guide RNA, trans-cleavage of ssDNA, such as an ssDNA reporter, by the programmable nuclease is activated. Detection of trans-cleavage of ssDNA can be used to determine a target DNA is in a sample. In some embodiments, the programmable nuclease is a programmable nickase. In further embodiments, the programmable nuclease is a programmable DNA nickase.

The programmable nickases disclosed herein may exhibit cis-cleavage activity or target cleavage activity. Target cleavage activity may refer to the cleavage of a target nucleic acid by the programmable nickase. In some cases, the cis-cleavage activity results in double-stranded breaks in the target nucleic acids. In some cases, the cis-cleavage activity results in single-stranded breaks in the target nucleic acids (nickase activity). In some cases, the cis-cleavage activity produces a mixture of double- and single-stranded breaks in the target nucleic acids. In further cases, the rates of cis-cleavage double- and single-strand break formation may be dependent on the sequence of the guide nucleic acid. In some cases, the ratio of cis-cleavage double- and single-strand break formation may be dependent on the sequence of the guide nucleic acid.

### Reagents for Nicking Target Nucleic Acids and Detection of Target Nucleic Acids

A number of reagents are consistent with the compositions and methods disclosed herein. The reagents described herein may be used for nicking target nucleic acids and for detection of target nucleic acids. The reagents disclosed herein can include programmable nickases, guide nucleic acids, target nucleic acids, and buffers. As described herein, target nucleic acid comprising DNA or RNA may be modified or detected (e.g., the target DNA hybridizes to the guide nucleic) using a programmable nickase (e.g., a Cas14a, a Cas14b, or a Cas14e disclosed herein) and other reagents disclosed herein. As described herein, target nucleic acids comprising DNA may be an amplicon of a nucleic acid of interest and the amplicon can be detected (e.g., the target DNA hybridizes to the guide nucleic) using a programmable nickase and other reagents disclosed herein. Additionally, detection of multiple target nucleic acids is possible using two or more programmable nickases or a programmable nickase with a non-nickase programmable nuclease complexed to guide nucleic acids that target the multiple target nucleic acids, wherein the programmable nucleases exhibit different sequence-independent cleavage of the nucleic acid of a reporter (e.g., cleavage of an RNA reporter by a first programmable nuclease and cleavage of a DNA reporter by a second programmable nuclease).

### Programmable Nickases

In some embodiments, the programmable nickase of the present disclosure (e.g., a Cas14) is especially useful for genome editing and use in a DETECTR assay due to its small size. The smaller nature of these proteins allows for them to be more easily packaged and delivered with higher efficiency in the context of genome editing and more readily incorporated as a reagent in an assay. In some embodiments, the programmable nickase of the present disclosure are from 400 to 800 amino acid residues long, from 400 to 420 amino acid residues long, from 420 to 440 amino acid residues long, from 440 to 460 amino acid residues long, from 460 to 480 amino acid residues long, from 480 to 500 amino acid residues long, from 500 to 520 amino acid residues long, from 520 to 540 amino acid residues long, from 540 to 560 amino acid residues long, from 560 to 580 amino acid residues long, from 580 to 600 amino acid residues long, from 600 to 620 amino acid residues long, from 620 to 640 amino acid residues long, from 640 to 660 amino acid residues long, from 660 to 680 amino acid residues long, from 680 to 700 amino acid residues long, from 700 to 720 amino acid residues long, from 720 to 740 amino acid residues long, from 740 to 760 amino acid residues long, from 760 to 780 amino acid residues long, from 780 to 800 amino acid residues long, from 400 to 500 amino acid residues long, from 500 to 600 amino acid residues long, from 600 to 700 amino acid residues long, from 700 to 800 amino acid residues long, from 450 to 550 amino acid residues long, from 550 to 650 amino acid residues long, from 650 to 750 amino acid residues long, or from 750 to 800 amino acid residues long. In some embodiments, the programmable nickase of the present disclosure has a length from 350 to 900 amino acids. In some embodiments, the programmable nickase of the present disclosure has a length from 500 to 550 amino acids. In preferred embodiments, the programmable nickase of the present disclosure has a length of from 480 to 550 amino acid residues.

In some embodiments, the Type V CRISPR/Cas enzyme is a programmable Cas14 nuclease. Cas14 can be referred to as CasZ. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a target nucleic acid. A programmable Cas14 nuclease can be a Cas14a protein, a Cas14b protein, a Cas14c protein, a Cas14d protein, a Cas14e protein, a Cas 14f protein, a Cas14g protein, a Cas14h protein, Cas14j, Cas14k, Cas14l, or a Cas14u protein.

It is important to note that Cas14 is short compared to previously identified CRISPR-Cas endonucleases, and thus use of this protein as an alternative provides the advantage that the nucleotide sequence encoding the protein is relatively short. This is useful, for example, in cases where a nucleic acid encoding the Cas14 protein is desirable, e.g., in situations that employ a viral vector (e.g., an AAV vector), for delivery to a cell such as a eukaryotic cell (e.g., mammalian cell, human cell, mouse cell, in vitro, ex vivo, in vivo) for research and/or clinical applications. In addition, in their natural context, the Cas14-encoding DNA sequences are present in loci that also have a Cast protein.

Cas14s presented in TABLE 1 or variants thereof having at least 70%, at least 75%, at least 80%, at least 85%, at least 90%, at least 92%, at least 95%, at least 97%, at least 99%, or 100% sequence identity with any one of SEQ ID NO: 1-SEQ ID NO: 91 or SEQ ID NO: 170 can exhibit nicking activity. “Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

Exemplary programmable nickases (e.g., Cas14a, Cas14b, or Cas14e disclosed herein) of the present disclosure have at least 70%, at least 75%, at least 80%, at least 85%, at least 90%, at least 92%, at least 95%, at least 97%, at least 99%, or 100% sequence identity with any one of SEQ ID NO: 1-SEQ ID NO: 91 or SEQ ID NO: 170. An exemplary programmable nickase consistent with the compositions and methods disclosed herein has at least 70%, at least 75%, at least 80%, at least 85%, at least 90%, at least 92%, at least 95%, at least 97%, at least 99%, or 100% sequence identity to SEQ ID NO: 1. An exemplary programmable nickase consistent with the compositions and methods disclosed herein has at least 70%, at least 75%, at least 80%, at least 85%, at least 90%, at least 92%, at least 95%, at least 97%, at least 99%, or 100% sequence identity to SEQ ID NO: 10. An exemplary programmable nickase consistent with the compositions and methods disclosed herein has at least 70%, at least 75%, at least 80%, at least 85%, at least 90%, at least 92%, at least 95%, at least 97%, at least 99%, or 100% sequence identity to SEQ ID NO: 11. An exemplary programmable nickase consistent with the compositions and methods disclosed herein has at least 70%, at least 75%, at least 80%, at least 85%, at least 90%, at least 92%, at least 95%, at least 97%, at least 99%, or 100% sequence identity to SEQ ID NO: 17. An exemplary programmable nickase consistent with the compositions and methods disclosed herein has at least 70%, at least 75%, at least 80%, at least 85%, at least 90%, at least 92%, at least 95%, at least 97%, at least 99%, or 100% sequence identity to SEQ ID NO: 33.

TABLE 1
Cas14 Sequences

| SEQ | |
|---|---|
| ID | |
| NO | Sequence |
| SEQ | MEVQKTVMKTLSLRILRPLYSQEIEKEIKEEKERRKQAGGTGELDGGFYKKLEKKHS |
| ID | EMFSFDRLNLLLNQLQREIAKVYNHAISELYIATIAQGNKSNKHYISSIVYNRAYGYF |
| NO: | YNAYIALGICSKVEANFRSNELLTQQSALPTAKSDNFPIVLHKQKGAEGEDGGFRIST |
| 1 | EGSDLIFEIPIPFYEYNGENRKEPYKWVKKGGQKPVLKLILSTFRRQRNKGWAKDEG |
| | TDAEIRKVTEGKYQVSQIEINRGKKLGEHQKWFANFSIEQPIYERKPNRSIVGGLDVG |
| | IRSPLVCAINNSFSRYSVDSNDVFKFSKQVFAFRRRLLSKNSLKRKGHGAAHKLEPIT |
| | EMTEKNDKFRKKIIERWAKEVTNFFVKNQVGIVQIEDLSTMKDREDHFFNQYLRGF |
| | WPYYQMQTLIENKLKEYGIEVKRVQAKYTSQLCSNPNCRYWNNYFNFEYRKVNKF |
| | PKFKCEKCNLEISADYNAARNLSTPDIEKFVAKATKGINLPEK |
| SEQ | MEEAKTVSKTLSLRILRPLYSAEIEKEIKEEKERRKQGGKSGELDSGFYKKLEKKHT |
| ID | QMFGWDKLNLMLSQLQRQIARVFNQSISELYIETVIQGKKSNKHYTSKIVYNRAYSV |
| NO: | FYNAYLALGITSKVEANFRSTELLMQKSSLPTAKSDNFPILLHKQKGVEGEEGGFKIS |
| 2 | ADGNDLIFEIPIPFYEYDSANKKEPFKWIKKGGQKPTIKLILSTFRRQRNKGWAKDEG |
| | TDAEIRKVIEGKYQVSHIEINRGKKLGDHQKWFVNFTIEQPIYERKLDKNIIGGIDVGI |
| | KSPLVCAVNNSFARYSVDSNDVLKFSKQAFAFRRRLLSKNSLKRSGHGSKNKLDPIT |
| | RMTEKNDRFRKKIIERWAKEVTNFFIKNQVGTVQIEDLSTMKDRQDNFFNQYLRGF |
| | WPYYQMQNLIENKLKEYGIETKRIKARYTSQLCSNPSCRHWNSYFSFDHRKTNNFP |
| | KFKCEKCALEISADYNAARNISTPDIEKFVAKATKGINLPDKNENVILE |
| SEQ | MAKNTITKTLKLRIVRPYNSAEVEKIVADEKNNREKIALEKNKDKVKEACSKHLKV |
| ID | AAYCTTQVERNACLFCKARKLDDKFYQKLRGQFPDAVFWQEISEIFRQLQKQAAEI |
| NO: | YNQSLIELYYEIFIKGKGIANASSVEHYLSDVCYTRAAELFKNAAIASGLRSKIKSNFR |
| 3 | LKELKNMKSGLPTTKSDNFPIPLVKQKGGQYTGFEISNHNSDFIIKIPFGRWQVKKEI |
| | DKYRPWEKFDFEQVQKSPKPISLLLSTQRRKRNKGWSKDEGTEAEIKKVMNGDYQT |
| | SYIEVKRGSKIGEKSAWMLNLSIDVPKIDKGVDPSIIGGIDVGVKSPLVCAINNAFSRY |
| | SISDNDLFHFNKKMFARRRILLKKNRHKRAGHGAKNKLKPITILTEKSERFRKKLIER |
| | WACEIADFFIKNKVGTVQMENLESMKRKEDSYFNIRLRGFWPYAEMQNKIEFKLKQ |
| | YGIEIRKVAPNNTSKTCSKCGHLNNYFNFEYRKKNKFPHFKCEKCNFKENADYNAA |
| | LNISNPKLKSTKEEP |
| SEQ | MERQKVPQIRKIVRVVPLRILRPKYSDVIENALKKFKEKGDDTNTNDFWRAIRDRDT |
| ID | EFFRKELNFSEDEINQLERDTLFRVGLDNRVLFSYFDFLQEKLMKDYNKIISKLFINR |
| NO: | QSKSSFENDLTDEEVEELIEKDVTPFYGAYIGKGIKSVIKSNLGGKFIKSVKIDRETKK |
| 4 | VTKLTAINIGLMGLPVAKSDTFPIKIIKTNPDYITFQKSTKENLQKIEDYETGIEYGDLL |
| | VQITIPWFKNENKDFSLIKTKEAIEYYKLNGVGKKDLLNINLVLTTYHIRKKKSWQID |
| | GSSQSLVREMANGELEEKWKSFFDTFIKKYGDEGKSALVKRRVNKKSRAKGEKGR |
| | ELNLDERIKRLYDSIKAKSFPSEINLIPENYKWKLHFSIEIPPMVNDIDSNLYGGIDFGE |
| | QNIATLCVKNIEKDDYDFLTIYGNDLLKHAQASYARRRIMRVQDEYKARGHGKSRK |
| | TKAQEDYSERMQKLRQKITERLVKQISDFFLWRNKFHMAVCSLRYEDLNTLYKGES |
| | VKAKRMRQFINKQQLFNGIERKLKDYNSEIYVNSRYPHYTSRLCSKCGKLNLYFDFL |
| | KFRTKNIIIRKNPDGSEIKYMPFFICEFCGWKQAGDKNASANIADKDYQDKLNKEKE |
| | FCNIRKPKSKKEDIGEENEEERDYSRRFNRNSFIYNSLKKDNKLNQEKLFDEWKNQL |
| | KRKIDGRNKFEPKEYKDRFSYLFAYYQEIIKNESES |
| SEQ | MVPTELITKTLQLRVIRPLYFEEIEKELAELKEQKEKEFEETNSLLLESKKIDAKSLKK |
| ID | LKRKARSSAAVEFWKIAKEKYPDILTKPEMEFIFSEMQKMMARFYNKSMTNIFIEMN |
| NO: | NDEKVNPLSLISKASTEANQVIKCSSISSGLNRKIAGSINKTKFKQVRDGLISLPTART |
| 5 | ETFPISFYKSTANKDEIPISKINLPSEEEADLTITLPFPFFEIKKEKKGQKAYSYFNIIEKS |
| | GRSNNKIDLLLSTHRRQRRKGWKEEGGTSAEIRRLMEGEFDKEWEIYLGEAEKSEK |
| | AKNDLIKNMTRGKLSKDIKEQLEDIQVKYFSDNNVESWNDLSKEQKQELSKLRKKK |
| | VEELKDWKHVKEILKTRAKIGWVELKRGKRQRDRNKWFVNITITRPPFINKELDDT |
| | KFGGIDLGVKVPFVCAVHGSPARLIIKENEILQFNKMVSARNRQITKDSEQRKGRGK |
| | KNKFIKKEIFNERNELFRKKIIERWANQIVKFFEDQKCATVQIENLESFDRTSYK |
| SEQ | MKSDTKDKKIIIHQTKTLSLRIVKPQSIPMEEFTDLVRYHQMIIFPVYNNGAIDLYKKL |
| ID | FKAKIQKGNEARAIKYFMNKIVYAPIANTVKNSYIALGYSTKMQSSFSGKRLWDLRF |
| NO: | GEATPPTIKADFPLPFYNQSGFKVSSENGEFIIGIPFGQYTKKTVSDIEKKTSFAWDKF |
| 6 | TLEDTTKKTLIELLLSTKTRKMNEGWKNNEGTEAEIKRVMDGTYQVTSLEILQRDDS |
| | WFVNFNIAYDSLKKQPDRDKIAGIHMGITRPLTAVIYNNKYRALSIYPNTVMHLTQK |
| | QLARIKEQRTNSKYATGGHGRNAKVTGTDTLSEAYRQRRKKIIEDWIASIVKFAINN |
| | EIGTIYLEDISNTNSFFAAREQKLIYLEDISNTNSFLSTYKYPISAISDTLQHKLEEKAIQ |
| | VIRKKAYYVNQICSLCGHYNKGFTYQFRRKNKFPKMKCQGCLEATSTEFNAAANV |
| | ANPDYEKLLIKHGLLQLKK |
| SEQ | MSTITRQVRLSPTPEQSRLLMAHCQQYISTVNVLVAAFDSEVLTGKVSTKDFRAALP |
| ID | SAVKNQALRDAQSVFKRSVELGCLPVLKKPHCQWNNQNWRVEGDQLILPICKDGK |
| NO: | TQQERFRCAAVALEGKAGILRIKKKRGKWIADLTVTQEDAPESSGSAIMGVDLGIKV |
| 7 | PAVAHIGGKGTRFFGNGRSQRSMRRRFYARRKTLQKAKKLRAVRKSKGKEARWM |
| | KTINHQLSRQIVNHAHALGVGTIKIEALQGIRKGTTRKSRGAAARKNNRMTNTWSFS |
| | QLTLFITYKAQRQGITVEQVDPAYTSQDCPACRARNGAQDRTYVCSECGWRGHRD |
| | TVGAINISRRAGLSGHRRGATGA |
| SEQ | MIAQKTIKIKLNPTKEQIIKLNSIIEEYIKVSNFTAKKIAEIQESFTDSGLTQGTCSECGK |
| ID | EKTYRKYHLLKKDNKLFCITCYKRKYSQFTLQKVEFQNKTGLRNVAKLPKTYYTN |
| NO: | AIRFASDTFSGFDEIIKKKQNRLNSIQNRLNFWKELLYNPSNRNEIKIKVVKYAPKTD |
| 8 | TREHPHYYSEAEIKGRIKRLEKQLKKFKMPKYPEFTSETISLQRELYSWKNPDELKIS |
| | SITDKNESMNYYGKEYLKRYIDLINSQTPQILLEKENNSFYLCFPITKNIEMPKIDDTF |
| | EPVGIDWGITRNIAVVSILDSKTKKPKFVKFYSAGYILGKRKHYKSLRKHFGQKKRQ |
| | DKINKLGTKEDRFIDSNIHKLAFLIVKEIRNHSNKPIILMENITDNREEAEKSMRQNIL |
| | LHSVKSRLQNYIAYKALWNNIPTNLVKPEHTSQICNRCGHQDRENRPKGSKLFKCV |
| | KCNYMSNADFNASINIARKFYIGEYEPFYKDNEKMKSGVNSISM |
| SEQ | LKLSEQENITTGVKFKLKLDKETSEGLNDYFDEYGKAINFAIKVIQKELAEDRFAGK |
| ID | VRLDENKKPLLNEDGKKIWDFPNEFCSCGKQVNRYVNGKSLCQECYKNKFTEYGIR |
| NO: | KRMYSAKGRKAEQDINIKNSTNKISKTHFNYAIREAFILDKSIKKQRKERFRRLREM |
| 9 | KKKLQEFIEIRDGNKILCPKIEKQRVERYIHPSWINKEKKLEDFRGYSMSNVLGKIKIL |
| | DRNIKREEKSLKEKGQINFKARRLMLDKSVKFLNDNKISFTISKNLPKEYELDLPEKE |
| | KRLNWLKEKIKIIKNQKPKYAYLLRKDDNFYLQYTLETEFNLKEDYSGIVGIDRGVS |
| | HIAVYTFVHNNGKNERPLFLNSSEILRLKNLQKERDRFLRRKHNKKRKKSNMRNIEK |
| | KIQLILHNYSKQIVDFAKNKNAFIVFEKLEKPKKNRSKMSKKSQYKLSQFTFKKLSD |
| | LVDYKAKREGIKVLYISPEYTSKECSHCGEKVNTQRPFNGNSSLFKCNKCGVELNAD |
| | YNASINIAKKGLNILNSTN |
| SEQ | MEESIITGVKFKLRIDKETTKKLNEYFDEYGKAINFAVKIIQKELADDRFAGKAKLDQ |
| ID | NKNPILDENGKKIYEFPDEFCSCGKQVNKYVNNKPFCQECYKIRFTENGIRKRMYSA |
| NO: | KGRKAEHKINILNSTNKISKTHFNYAIREAFILDKSIKKQRKKRNERLRESKKRLQQFI |
| 10 | DMRDGKREICPTIKGQKVDRFIHPSWITKDKKLEDFRGYTLSIINSKIKILDRNIKREE |
| | KSLKEKGQIIFKAKRLMLDKSIRFVGDRKVLFTISKTLPKEYELDLPSKEKRLNWLKE |
| | KIEIIKNQKPKYAYLLRKNIESEKKPNYEYYLQYTLEIKPELKDFYDGAIGIDRGINHI |
| | AVCTFISNDGKVTPPKFFSSGEILRLKNLQKERDRFLLRKHNKNRKKGNMRVIENKI |
| | NLILHRYSKQIVDMAKKLNASIVFEELGRIGKSRTKMKKSQRYKLSLFIFKKLSDLVD |
| | YKSRREGIRVTYVPPEYTSKECSHCGEKVNTQRPFNGNYSLFKCNKCGIQLNSDYNA |
| | SINIAKKGLKIPNST |
| SEQ | MPKQDLVTTGIKFKLDVDKETRKKLDDYFDEYGKAINFAVKIIQKNLKEDRFAGKIA |
| ID | LGEDKKPLLDKDGKKIYNYPNESCSCGNQVRRYVNAKPFCVDCYKLKFTENGIRKR |
| NO: | MYSARGRKADSDINIKNSTNKISKTHFNYAIREGFILDKSLKKQRSKRIKKLLELKRK |
| 11 | LQEFIDIRQGQMVLCPKIKNQRVDKFIHPSWLKRDKKLEEFRGYSLSVVEGKIKIFNR |
| | NILREEDSLRQRGHVNFKANRIMLDKSVRFLDGGKVNFNLNKGLPKEYLLDLPKKE |
| | NKLSWLNEKISLIKLQKPKYAYLLRREGSFFIQYTIENVPKTFSDYLGAIGIDRGISHIA |
| | VCTFVSKNGVNKAPVFFSSGEILKLKSLQKQRDLFLRGKHNKIRKKSNMRNIDNKIN |
| | LILHKYSRNIVNLAKSEKAFIVFEKLEKIKKSRFKMSKSLQYKLSQFTFKKLSDLVEY |
| | KAKIEGIKVDYVPPEYTSKECSHCGEKVDTQRPFNGNSSLFKCNKCRVQLNADYNA |
| | SINIAKKSLNISN |
| SEQ | MSKTTISVKLKIIDLSSEKKEFLDNYFNEYAKATTFCQLRIRRLLRNTHWLGKKEKSS |
| ID | KKWIFESGICDLCGENKELVNEDRNSGEPAKICKRCYNGRYGNQMIRKLFVSTKKR |
| NO: | EVQENMDIRRVAKLNNTHYHRIPEEAFDMIKAADTAEKRRKKNVEYDKKRQMEFIE |
| 12 | MFNDEKKRAARPKKPNERETRYVHISKLESPSKGYTLNGIKRKIDGMGKKIERAEKG |
| | LSRKKIFGYQGNRIKLDSNWVRFDLAESEITIPSLFKEMKLRITGPTNVHSKSGQIYFA |
| | EWFERINKQPNNYCYLIRKTSSNGKYEYYLQYTYEAEVEANKEYAGCLGVDIGCSK |
| | LAAAVYYDSKNKKAQKPIEIFTNPIKKIKMRREKLIKLLSRVKVRHRRRKLMQLSKT |
| | EPIIDYTCHKTARKIVEMANTAKAFISMENLETGIKQKQQARETKKQKFYRNMFLFR |
| | KLSKLIEYKALLKGIKIVYVKPDYTSQTCSSCGADKEKTERPSQAIFRCLNPTCRYYQ |
| | RDINADFNAAVNIAKKALNNTEVVTTLL |
| SEQ | MARAKNQPYQKLTTTTGIKFKLDLSEEEGKRFDEYFSEYAKAVNFCAKVIYQLRKN |
| ID | LKFAGKKELAAKEWKFEISNCDFCNKQKEIYYKNIANGQKVCKGCHRTNFSDNAIR |
| NO: | KKMIPVKGRKVESKFNIHNTTKKISGTHRHWAFEDAADIIESMDKQRKEKQKRLRR |
| 13 | EKRKLSYFFELFGDPAKRYELPKVGKQRVPRYLHKIIDKDSLTKKRGYSLSYIKNKIK |
| | ISERNIERDEKSLRKASPIAFGARKIKMSKLDPKRAFDLENNVFKIPGKVIKGQYKFF |
| | GTNVANEHGKKFYKDRISKILAGKPKYFYLLRKKVAESDGNPIFEYYVQWSIDTETP |
| | AITSYDNILGIDAGITNLATTVLIPKNLSAEHCSHCGNNHVKPIFTKFFSGKELKAIKIK |
| | SRKQKYFLRGKHNKLVKIKRIRPIEQKVDGYCHVVSKQIVEMAKERNSCIALEKLEK |
| | PKKSKFRQRRREKYAVSMFVFKKLATFIKYKAAREGIEIIPVEPEGTSYTCSHCKNAQ |
| | NNQRPYFKPNSKKSWTSMFKCGKCGIELNSDYNAAFNIAQKALNMTSA |
| SEQ | MDEKHFFCSYCNKELKISKNLINKISKGSIREDEAVSKAISIHNKKEHSLILGIKFKLFI |
| ID | ENKLDKKKLNEYFDNYSKAVTFAARIFDKIRSPYKFIGLKDKNTKKWTFPKAKCVF |
| NO: | CLEEKEVAYANEKDNSKICTECYLKEFGENGIRKKIYSTRGRKVEPKYNIFNSTKELS |
| 14 | STHYNYAIRDAFQLLDALKKQRQKKLKSIFNQKLRLKEFEDIFSDPQKRIELSLKPHQ |
| | REKRYIHLSKSGQESINRGYTLRFVRGKIKSLTRNIEREEKSLRKKTPIHFKGNRLMIF |
| | PAGIKFDFASNKVKISISKNLPNEFNFSGTNVKNEHGKSFFKSRIELIKTQKPKYAYVL |
| | RKIKREYSKLRNYEIEKIRLENPNADLCDFYLQYTIETESRNNEEINGIIGIDRGITNLA |
| | CLVLLKKGDKKPSGVKFYKGNKILGMKIAYRKHLYLLKGKRNKLRKQRQIRAIEPK |
| | INLILHQISKDIVKIAKEKNFAIALEQLEKPKKARFAQRKKEKYKLALFTFKNLSTLIE |
| | YKSKREGIPVIYVPPEKTSQMCSHCAINGDEHVDTQRPYKKPNAQKPSYSLFKCNKC |
| | GIELNADYNAAFNIAQKGLKTLMLNHSH |
| SEQ | MLQTLLVKLDPSKEQYKMLYETMERFNEACNQIAETVFAIHSANKIEVQKTVYYPIR |
| ID | EKFGLSAQLTILAIRKVCEAYKRDKSIKPEFRLDGALVYDQRVLSWKGLDKVSLVTL |
| NO: | QGRQIIPIKFGDYQKARMDRIRGQADLILVKGVFYLCVVVEVSEESPYDPKGVLGVD |
| 15 | LGIKNLAVDSDGEVHSGEQTTNTRERLDSLKARLQSKGTKSAKRHLKKLSGRMAKF |
| | SKDVNHCISKKLVAKAKGTLMSIALEDLQGIRDRVTVRKAQRRNLHTWNFGLLRM |
| | FVDYKAKIAGVPLVFVDPRNTSRTCPSCGHVAKANRPTRDEFRCVSCGFAGAADHI |
| | AAMNIAFRAEVSQPIVTRFFVQSQAPSFRVG |
| SEQ | MDEEPDSAEPNLAPISVKLKLVKLDGEKLAALNDYFNEYAKAVNFCELKMQKIRKN |
| ID | LVNIRGTYLKEKKAWINQTGECCICKKIDELRCEDKNPDINGKICKKCYNGRYGNQ |
| NO: | MIRKLFVSTNKRAVPKSLDIRKVARLHNTHYHRIPPEAADIIKAIETAERKRRNRILFD |
| 16 | ERRYNELKDALENEEKRVARPKKPKEREVRYVPISKKDTPSKGYTMNALVRKVSG |
| | MAKKIERAKRNLNKRKKIEYLGRRILLDKNWVRFDFDKSEISIPTMKEFFGEMRFEIT |
| | GPSNVMSPNGREYFTKWFDRIKAQPDNYCYLLRKESEDETDFYLQYTWRPDAHPK |
| | KDYTGCLGIDIGGSKLASAVYFDADKNRAKQPIQIFSNPIGKWKTKRQKVIKVLSKA |
| | AVRHKTKKLESLRNIEPRIDVHCHRIARKIVGMALAANAFISMENLEGGIREKQKAK |
| | ETKKQKFSRNMFVFRKLSKLIEYKALMEGVKVVYIVPDYTSQLCSSCGTNNTKRPK |
| | QAIFMCQNTECRYFGKNINADFNAAINIAKKALNRKDIVRELS |
| SEQ | MEKNNSEQTSITTGIKFKLKLDKETKEKLNNYFDEYGKAINFAVRIIQMQLNDDRLA |
| ID | GKYKRDEKGKPILGEDGKKILEIPNDFCSCGNQVNHYVNGVSFCQECYKKRFSENGI |
| NO: | RKRMYSAKGRKAEQDINIKNSTNKISKTHFNYAIREAFNLDKSIKKQREKRFKKLKD |
| 17 | MKRKLQEFLEIRDGKRVICPKIEKQKVERYIHPSWINKEKKLEEFRGYSLSIVNSKIKS |
| | FDRNIQREEKSLKEKGQINFKAQRLMLDKSVKFLKDNKVSFTISKELPKTFELDLPKK |
| | EKKLNWLNEKLEIIKNQKPKYAYLLRKENNIFLQYTLDSIPEIHSEYSGAVGIDRGVS |
| | HIAVYTFLDKDGKNERPFFLSSSGILRLKNLQKERDKFLRKKHNKIRKKGNMRNIEQ |
| | KINLILHEYSKQIVNFAKDKNAFIVFELLEKPKKSRERMSKKIQYKLSQFTFKKLSDL |
| | VDYKAKREGIKVIYVEPAYTSKDCSHCGERVNTQRPFNGNFSLFKCNKCGIVLNSDY |
| | NASLNIARKGLNISAN |
| SEQ | MAEEKFFFCEKCNKDIKIPKNYINKQGAEEKARAKHEHRVHALILGIKFKIYPKKEDI |
| ID | SKLNDYFDEYAKAVTFTAKIVDKLKAPFLFAGKRDKDTSKKKWVFPVDKCSFCKE |
| NO: | KTEINYRTKQGKNICNSCYLTEFGEQGLLEKIYATKGRKVSSSFNLFNSTKKLTGTH |
| 18 | NNYVVKESLQLLDALKKQRSKRLKKLSNTRRKLKQFEEMFEKEDKRFQLPLKEKQR |
| | ELRFIHVSQKDRATEFKGYTMNKIKSKIKVLRRNIEREQRSLNRKSPVFFRGTRIRLSP |
| | SVQFDDKDNKIKLTLSKELPKEYSFSGLNVANEHGRKFFAEKLKLIKENKSKYAYLL |
| | RRQVNKNNKKPIYDYYLQYTVEFLPNIITNYNGILGIDRGINTLACIVLLENKKEKPSF |
| | VKFFSGKGILNLKNKRRKQLYFLKGVHNKYRKQQKIRPIEPRIDQILHDISKQIIDLAK |
| | EKRVAISLEQLEKPQKPKFRQSRKAKYKLSQFNFKTLSNYIDYKAKKEGIRVIYIAPE |
| | MTSQNCSRCAMKNDLHVNTQRPYKNTSSLFKCNKCGVELNADYNAAFNIAQKGLK |
| | ILNS |
| SEQ | MISLKLKLLPDEEQKKLLDEMFWKWASICTRVGFGRADKEDLKPPKDAEGVWFSLT |
| ID | QLNQANTDINDLREAMKHQKHRLEYEKNRLEAQRDDTQDALKNPDRREISTKRKD |
| NO: | LFRPKASVEKGFLKLKYHQERYWVRRLKEINKLIERKTKTLIKIEKGRIKFKATRITL |
| 19 | HQGSFKIRFGDKPAFLIKALSGKNQIDAPFVVVPEQPICGSVVNSKKYLDEITTNFLA |
| | YSVNAMLFGLSRSEEMLLKAKRPEKIKKKEEKLAKKQSAFENKKKELQKLLGRELT |
| | QQEEAIIEETRNQFFQDFEVKITKQYSELLSKIANELKQKNDFLKVNKYPILLRKPLK |
| | KAKSKKINNLSPSEWKYYLQFGVKPLLKQKSRRKSRNVLGIDRGLKHLLAVTVLEP |
| | DKKTFVWNKLYPNPITGWKWRRRKLLRSLKRLKRRIKSQKHETIHENQTRKKLKSL |
| | QGRIDDLLHNISRKIVETAKEYDAVIVVEDLQSMRQHGRSKGNRLKTLNYALSLFDY |
| | ANVMQLIKYKAGIEGIQIYDVKPAGTSQNCAYCLLAQRDSHEYKRSQENSKIGVCL |
| | NPNCQNHKKQIDADLNAARVIASCYALKINDSQPFGTRKRFKKRTTN |
| SEQ | METLSLKLKLNPSKEQLLVLDKMFWKWASICTRLGLKKAEMSDLEPPKDAEGVWF |
| ID | SKTQLNQANTDVNDLRKAMQHQGKRIEYELDKVENRRNEIQEMLEKPDRRDISPNR |
| NO: | KDLFRPKAAVEKGYLKLKYHKLGYWSKELKTANKLIERKRKTLAKIDAGKMKFKP |
| 20 | TRISLHTNSFRIKFGEEPKIALSTTSKHEKIELPLITSLQRPLKTSCAKKSKTYLDAAIL |
| | NFLAYSTNAALFGLSRSEEMLLKAKKPEKIEKRDRKLATKRESFDKKLKTLEKLLER |
| | KLSEKEKSVFKRKQTEFFDKFCITLDETYVEALHRIAEELVSKNKYLEIKKYPVLLRK |
| | PESRLRSKKLKNLKPEDWTYYIQFGFQPLLDTPKPIKTKTVLGIDRGVRHLLAVSIFD |
| | PRTKTFTFNRLYSNPIVDWKWRRRKLLRSIKRLKRRLKSEKHVHLHENQFKAKLRSL |
| | EGRIEDHFHNLSKEIVDLAKENNSVIVVENLGGMRQHGRGRGKWLKALNYALSHF |
| | DYAKVMQLIKYKAELAGVFVYDVAPAGTSINCAYCLLNDKDASNYTRGKVINGKK |
| | NTKIGECKTCKKEFDADLNAARVIALCYEKRLNDPQPFGTRKQFKPKKP |
| SEQ | MKALKLQLIPTRKQYKILDEMFWKWASLANRVSQKGESKETLAPKKDIQKIQFNAT |
| ID | QLNQIEKDIKDLRGAMKEQQKQKERLLLQIQERRSTISEMLNDDNNKERDPHRPLNF |
| NO: | RPKGWRKFHTSKHWVGELSKILRQEDRVKKTIERIVAGKISFKPKRIGIWSSNYKINF |
| 21 | FKRKISINPLNSKGFELTLMTEPTQDLIGKNGGKSVLNNKRYLDDSIKSLLMFALHSR |
| | FFGLNNTDTYLLGGKINPSLVKYYKKNQDMGEFGREIVEKFERKLKQEINEQQKKII |
| | MSQIKEQYSNRDSAFNKDYLGLINEFSEVFNQRKSERAEYLLDSFEDKIKQIKQEIGE |
| | SLNISDWDFLIDEAKKAYGYEEGFTEYVYSKRYLEILNKIVKAVLITDIYFDLRKYPIL |
| | LRKPLDKIKKISNLKPDEWSYYIQFGYDSINPVQLMSTDKFLGIDRGLTHLLAYSVFD |
| | KEKKEFIINQLEPNPIMGWKWKLRKVKRSLQHLERRIRAQKMVKLPENQMKKKLKS |
| | IEPKIEVHYHNISRKIVNLAKDYNASIVVESLEGGGLKQHGRKKNARNRSLNYALSL |
| | FDYGKIASLIKYKADLEGVPMYEVLPAYTSQQCAKCVLEKGSFVDPEIIGYVEDIGIK |
| | GSLLDSLFEGTELSSIQVLKKIKNKIELSARDNHNKEINLILKYNFKGLVIVRGQDKEE |
| | IAEHPIKEINGKFAILDFVYKRGKEKVGKKGNQKVRYTGNKKVGYCSKHGQVDAD |
| | LNASRVIALCKYLDINDPILFGEQRKSFK |
| SEQ | MVTRAIKLKLDPTKNQYKLLNEMFWKWASLANRFSQKGASKETLAPKDGTQKIQF |
| ID | NATQLNQIKKDVDDLRGAMEKQGKQKERLLIQIQERLLTISEILRDDSKKEKDPHRP |
| NO: | QNFRPFGWRRFHTSAYWSSEASKLTRQVDRVRRTIERIKAGKINFKPKRIGLWSSTY |
| 22 | KINFLKKKINISPLKSKSFELDLITEPQQKIIGKEGGKSVANSKKYLDDSIKSLLIFAIKS |
| | RLFGLNNKDKPLFENIITPNLVRYHKKGQEQENFKKEVIKKFENKLKKEISQKQKEII |
| | FSQIERQYENRDATFSEDYLRAISEFSEIFNQRKKERAKELLNSFNEKIRQLKKEVNG |
| | NISEEDLKILEVEAEKAYNYENGFIEWEYSEQFLGVLEKIARAVLISDNYFDLKKYPI |
| | LIRKPTNKSKKITNLKPEEWDYYIQFGYGLINSPMKIETKNFMGIDRGLTHLLAYSIF |
| | DRDSEKFTINQLELNPIKGWKWKLRKVKRSLQHLERRMRAQKGVKLPENQMKKRL |
| | KSIEPKIESYYHNLSRKIVNLAKANNASIVVESLEGGGLKQHGRKKNSRHRALNYAL |
| | SLFDYGKIASLIKYKSDLEGVPMYEVLPAYTSQQCAKCVLKKGSFVEPEIIGYIEEIGF |
| | KENLLTLLFEDTGLSSVQVLKKSKNKMTLSARDKEGKMVDLVLKYNFKGLVISQEK |
| | KKEEIVEFPIKEIDGKFAVLDSAYKRGKERISKKGNQKLVYTGNKKVGYCSVHGQV |
| | DADLNASRVIALCKYLGINEPIVFGEQRKSFK |
| SEQ | LDLITEPIQPHKSSSLRSKEFLEYQISDFLNFSLHSLFFGLASNEGPLVDFKIYDKIVIPK |
| ID | PEERFPKKESEEGKKLDSFDKRVEEYYSDKLEKKIERKLNTEEKNVIDREKTRIWGE |
| NO: | VNKLEEIRSIIDEINEIKKQKHISEKSKLLGEKWKKVNNIQETLLSQEYVSLISNLSDEL |
| 23 | TNKKKELLAKKYSKFDDKIKKIKEDYGLEFDENTIKKEGEKAFLNPDKFSKYQFSSS |
| | YLKLIGEIARSLITYKGFLDLNKYPIIFRKPINKVKKIHNLEPDEWKYYIQFGYEQINN |
| | PKLETENILGIDRGLTHILAYSVFEPRSSKFILNKLEPNPIEGWKWKLRKLRRSIQNLE |
| | RRWRAQDNVKLPENQMKKNLRSIEDKVENLYHNLSRKIVDLAKEKNACIVFEKLEG |
| | QGMKQHGRKKSDRLRGLNYKLSLFDYGKIAKLIKYKAEIEGIPIYRIDSAYTSQNCA |
| | KCVLESRRFAQPEEISCLDDFKEGDNLDKRILEGTGLVEAKIYKKLLKEKKEDFEIEE |
| | DIAMFDTKKVIKENKEKTVILDYVYTRRKEIIGTNHKKNIKGIAKYTGNTKIGYCMK |
| | HGQVDADLNASRTIALCKNFDINNPEIWK |
| SEQ | MSDESLVSSEDKLAIKIKIVPNAEQAKMLDEMFKKWSSICNRISRGKEDIETLRPDEG |
| ID | KELQFNSTQLNSATMDVSDLKKAMARQGERLEAEVSKLRGRYETIDASLRDPSRRH |
| NO: | TNPQKPSSFYPSDWDISGRLTPRFHTARHYSTELRKLKAKEDKMLKTINKIKNGKIVF |
| 24 | KPKRITLWPSSVNMAFKGSRLLLKPFANGFEMELPIVISPQKTADGKSQKASAEYMR |
| | NALLGLAGYSINQLLFGMNRSQKMLANAKKPEKVEKFLEQMKNKDANFDKKIKAL |
| | EGKWLLDRKLKESEKSSIAVVRTKFFKSGKVELNEDYLKLLKHMANEILERDGFVN |
| | LNKYPILSRKPMKRYKQKNIDNLKPNMWKYYIQFGYEPIFERKASGKPKNIMGIDRG |
| | LTHLLAVAVFSPDQQKFLFNHLESNPIMHWKWKLRKIRRSIQHMERRIRAEKNKHIH |
| | EAQLKKRLGSIEEKTEQHYHIVSSKIINWAIEYEAAIVLESLSHMKQRGGKKSVRTRA |
| | LNYALSLFDYEKVARLITYKARIRGIPVYDVLPGMTSKTCATCLLNGSQGAYVRGLE |
| | TTKAAGKATKRKNMKIGKCMVCNSSENSMIDADLNAARVIAICKYKNLNDPQPAG |
| | SRKVFKRF |
| SEQ | MLALKLKIMPTEKQAEILDAMFWKWASICSRIAKMKKKVSVKENKKELSKKIPSNS |
| ID | DIWFSKTQLCQAEVDVGDHKKALKNFEKRQESLLDELKYKVKAINEVINDESKREI |
| NO: | DPNNPSKFRIKDSTKKGNLNSPKFFTLKKWQKILQENEKRIKKKESTIEKLKRGNIFF |
| 25 | NPTKISLHEEEYSINFGSSKLUNCFYKYNKKSGINSDQLENKFNEFQNGLNIICSPLQ |
| | PIRGSSKRSFEFIRNSIINFLMYSLYAKLFGIPRSVKALMKSNKDENKLKLEEKLKKK |
| | KSSFNKTVKEFEKMIGRKLSDNESKILNDESKKFFEIIKSNNKYIPSEEYLKLLKDISEE |
| | IYNSNIDFKPYKYSILIRKPLSKFKSKKLYNLKPTDYKYYLQLSYEPFSKQLIATKTIL |
| | GIDRGLKHLLAVSVFDPSQNKFVYNKLIKNPVFKWKKRYHDLKRSIRNRERRIRALT |
| | GVHIHENQLIKKLKSMKNKINVLYHNVSKNIVDLAKKYESTIVLERLENLKQHGRSK |
| | GKRYKKLNYVLSNFDYKKIESLISYKAKKEGVPVSNINPKYTSKTCAKCLLEVNQLS |
| | ELKNEYNRDSKNSKIGICNIHGQIDADLNAARVIALCYSKNLNEPHFK |
| SEQ | VINLFGYKFALYPNKTQEELLNKHLGECGWLYNKAIEQNEYYKADSNIEEAQKKFE |
| ID | LLPDKNSDEAKVLRGNISKDNYVYRTLVKKKKSEINVQIRKAVVLRPAETIRNLAKV |
| NO: | KKKGLSVGRLKFIPIREWDVLPFKQSDQIRLEENYLILEPYGRLKFKMHRPLLGKPKT |
| 26 | FCIKRTATDRWTISFSTEYDDSNMRKNDGGQVGIDVGLKTHLRLSNENPDEDPRYPN |
| | PKIWKRYDRRLTILQRRISKSKKLGKNRTRLRLRLSRLWEKIRNSRADLIQNETYEIL |
| | SENKLIAIEDLNVKGMQEKKDKKGRKGRTRAQEKGLHRSISDAAFSEFRRVLEYKA |
| | KRFGSEVKPVSAIDSSKECHNCGNKKGMPLESRIYECPKCGLKIDRDLNSAKVILAR |
| | ATGVRPGSNARADTKISATAGASVQTEGTVSEDFRQQMETSDQKPMQGEGSKEPPM |
| | NPEHKSSGRGSKHVNIGCKNKVGLYNEDENSRSTEKQIMDENRSTTEDMVEIGALH |
| | SPVLTT |
| SEQ | MIASIDYEAVSQALIVFEFKAKGKDSQYQAIDEAIRSYRFIRNSCLRYWMDNKKVGK |
| ID | YDLNKYCKVLAKQYPFANKLNSQARQSAAECSWSAISRFYDNCKRKVSGKKGFPK |
| NO: | FKKHARSVEYKTSGWKLSENRKAITFTDKNGIGKLKLKGTYDLHFSQLEDMKRVRL |
| 27 | VRRADGYYVQFCISVDVKVETEPTGKAIGLDVGIKYFLADSSGNTIENPQFYRKAEK |
| | KLNRANRRKSKKYIRGVKPQSKNYHKARCRYARKHLRVSRQRKEYCKRVAYCVIH |
| | SNDVVAYEDLNVKGMVKNRHLAKSISDVAWSTFRHWLEYFAIKYGKLTIPVAPHN |
| | TSQNCSNCDKKVPKSLSTRTHICHHCGYSEDRDVNAAKNILKKALSTVGQTGSLKL |
| | GEIEPLLVLEQSCTRKFDL |
| SEQ | LAEENTLHLTLAMSLPLNDLPENRTRSELWRRQWLPQKKLSLLLGVNQSVRKAAA |
| ID | DCLRWFEPYQELLWWEPTDPDGKKLLDKEGRPIKRTAGHMRVLRKLEEIAPFRGYQ |
| NO: | LGSAVKNGLRHKVADLLLSYAKRKLDPQFTDKTSYPSIGDQFPIVWTGAFVCYEQSI |
| 28 | TGQLYLYLPLFPRGSHQEDITNNYDPDRGPALQVFGEKEIARLSRSTSGLLLPLQFDK |
| | WGEATFIRGENNPPTWKATHRRSDKKWLSEVLLREKDFQPKRVELLVRNGRIFVNV |
| | ACEIPTKPLLEVENFMGVSFGLEHLVTVVVINRDGNVVHQRQEPARRYEKTYFARL |
| | ERLRRRGGPFSQELETFHYRQVAQIVEEALRFKSVPAVEQVGNIPKGRYNPRLNLRL |
| | SYWPFGKLADLTSYKAVKEGLPKPYSVYSATAKMLCSTCGAANKEGDQPISLKGPT |
| | VYCGNCGTRHNTGFNTALNLARRAQELFVKGVVAR |
| SEQ | MSQSLLKWHDMAGRDKDASRSLQKSAVEGVLLHLTASHRVALEMLEKSVSQTVA |
| ID | VTMEAAQQRLVIVLEDDPTKATSRKRVISADLQFTREEFGSLPNWAQKLASTCPEIA |
| NO: | TKYADKHINSIRIAWGVAKESTNGDAVEQKLQWQIRLLDVTMFLQQLVLQLADKA |
| 29 | LLEQIPSSIRGGIGQEVAQQVTSHIQLLDSGTVLKAELPTISDRNSELARKQWEDAIQT |
| | VCTYALPFSRERARILDPGKYAAEDPRGDRLINIDPMWARVLKGPTVKSLPLLFVSG |
| | SSIRIVKLTLPRKHAAGHKHTFTATYLVLPVSREWINSLPGTVQEKVQWWKKPDVL |
| | ATQELLVGKGALKKSANTLVIPISAGKKRFFNHILPALQRGFPLQWQRIVGRSYRRP |
| | ATHRKWFAQLTIGYTNPSSLPEMALGIHFGMKDILWWALADKQGNILKDGSIPGNSI |
| | LDFSLQEKGKIERQQKAGKNVAGKKYGKSLLNATYRVVNGVLEFSKGISAEHASQP |
| | IGLGLETIRFVDKASGSSPVNARHSNWNYGQLSGIFANKAGPAGFSVTEITLKKAQR |
| | DLSDAEQARVLAIEATKRFASRIKRLATKRKDDTLFV |
| SEQ | VEPVEKERFYYRTYTFRLDGQPRTQNLTTQSGWGLLTKAVLDNTKHYWEIVHHARI |
| ID | ANQPIVFENPVIDEQGNPKLNKLGQPRFWKRPISDIVNQLRALFENQNPYQLGSSLIQ |
| NO: | GTYWDVAENLASWYALNKEYLAGTATWGEPSFPEPHPLTEINQWMPLTFSSGKVV |
| 30 | RLLKNASGRYFIGLPILGENNPCYRMRTIEKLIPCDGKGRVTSGSLILFPLVGIYAQQH |
| | RRMTDICESIRTEKGKLAWAQVSIDYVREVDKRRRMRRTRKSQGWIQGPWQEVFIL |
| | RLVLAHKAPKLYKPRCFAGISLGPKTLASCVILDQDERVVEKQQWSGSELLSLIHQG |
| | EERLRSLREQSKPTWNAAYRKQLKSLINTQVFTIVTFLRERGAAVRLESIARVRKSTP |
| | APPVNFLLSHWAYRQITERLKDLAIRNGMPLTHSNGSYGVRFTCSQCGATNQGIKDP |
| | TKYKVDIESETFLCSICSHREIAAVNTATNLAKQLLDE |
| SEQ | MNDTETSETLTSHRTVCAHLHVVGETGSLPRLVEAALAELITLNGRATQALLSLAKN |
| ID | GLVLRRDKEENLIAAELTLPCRKNKYADVAAKAGEPILATRINNKGKLVTKKWYGE |
| NO: | GNSYHIVRFTPETGMFTVRVFDRYAFDEELLHLHSEVVFGSDLPKGIKAKTDSLPAN |
| 31 | FLQAVFTSFLELPFQGFPDIVVKPAMKQAAEQLLSYVQLEAGENQQAEYPDTNERDP |
| | ELRLVEWQKSLHELSVRTEPFEFVRARDIDYYAETDRRGNRFVNITPEWTKFAESPF |
| | ARRLPLKIPPEFCILLRRKTEGHAKIPNRIYLGLQIFDGVTPDSTLGVLATAEDGKLFW |
| | WHDHLDEFSNLEGKPEPKLKNKPQLLMVSLEYDREQRFEESVGGDRKICLVTLKET |
| | RNFRRGWNGRILGIHFQHNPVITWALMDHDAEVLEKGFIEGNAFLGKALDKQALNE |
| | YLQKGGKWVGDRSFGNKLKGITHTLASLIVRLAREKDAWIALEEISWVQKQSADSV |
| | ANHEIVEQPHHSLTR |
| SEQ | MNDTETSETLTSHRTVCAHLHVVGETGSLPRLVEAALAELITLNGRATQALLSLAKN |
| ID | GLVLRRDKEENLIAAELTLPCRKNKYADVAAKAGEPILATRINNKGKLVTKKWYGE |
| NO: | GNSYHIVRFTPETGMFTVRVFDRYAFDEELLHLHSEVVFGSDLPKGIKAKTDSLPAN |
| 32 | FLQAVFTSFLELPFQGFPDIVVKPAMKQAAEQLLSYVQLEAGENQQAEYPDTNERDP |
| | ELRLVEWQKSLHELSVRTEPFEFVRARDIDYYAETDRRGNRFVNITPEWTKFAESPF |
| | ARRLPLKIPPEFCILLRRKTEGHAKIPNRIYLGLQIFDGVTPDSTLGVLATAEDGKLFW |
| | WHDHLDEFSNLEGKPEPKLKNKPQLLMVSLEYDREQRFEESVGGDRKICLVTLKET |
| | RNFRRGRHGHTRTDRLPAGNTLWRADFATSAEVAAPKWNGRILGIHFQHNPVITWA |
| | LMDHDAEVLEKGFIEGNAFLGKALDKQALNEYLQKGGKWVGDRSFGNKLKGITHT |
| | LASLIVRLAREKDAWIALEEISWVQKQSADSVANRRFSMWNYSRLATLIEWLGTDI |
| | ATRDCGTAAPLAHKVSDYLTHFTCPECGACRKAGQKKEIADTVRAGDILTCRKCGF |
| | SGPIPDNFIAEFVAKKALERMLKKKPV |
| SEQ | MAKRNFGEKSEALYRAVRFEVRPSKEELSILLAVSEVLRMLFNSALAERQQVFTEFI |
| ID | ASLYAELKSASVPEEISEIRKKLREAYKEHSISLFDQINALTARRVEDEAFASVTRNW |
| NO: | QEETLDALDGAYKSFLSLRRKGDYDAHSPRSRDSGFFQKIPGRSGFKIGEGRIALSCG |
| 33 | AGRKLSFPIPDYQQGRLAETTKLKKFELYRDQPNLAKSGRFWISVVYELPKPEATTC |
| | QSEQVAFVALGASSIGVVSQRGEEVIALWRSDKHWVPKIEAVEERMKRRVKGSRG |
| | WLRLLNSGKRRMHMISSRQHVQDEREIVDYLVRNHGSHFVVTELVVRSKEGKLAD |
| | SSKPERGGSLGLNWAAQNTGSLSRLVRQLEEKVKEHGGSVRKHKLTLTEAPPARGA |
| | ENKLWMARKLRESFLKEV |
| SEQ | LAKNDEKELLYQSVKFEIYPDESKIRVLTRVSNILVLVWNSALGERRARFELYIAPLY |
| ID | EELKKFPRKSAESNALRQKIREGYKEHIPTFFDQLKKLLTPMRKEDPALLGSVPRAY |
| NO: | QEETLNTLNGSFVSFMTLRRNNDMDAKPPKGRAEDRFHEISGRSGFKIDGSEFVLST |
| 34 | KEQKLRFPIPNYQLEKLKEAKQIKKFTLYQSRDRRFWISIAYEIELPDQRPFNPEEVIYI |
| | AFGASSIGVISPEGEKVIDFWRPDKHWKPKIKEVENRMRSCKKGSRAWKKRAAARR |
| | KMYAMTQRQQKLNHREIVASLLRLGFHFVVTEYTVRSKPGKLADGSNPKRGGAPQ |
| | GFNWSAQNTGSFGEFILWLKQKVKEQGGTVQTFRLVLGQSERPEKRGRDNKIEMVR |
| | LLREKYLESQTIVV |
| SEQ | MAKGKKKEGKPLYRAVRFEIFPTSDQITLFLRVSKNLQQVWNEAWQERQSCYEQFF |
| ID | GSIYERIGQAKKRAQEAGFSEVWENEAKKGLNKKLRQQEISMQLVSEKESLLQELSI |
| NO: | AFQEHGVTLYDQINGLTARRIIGEFALIPRNWQEETLDSLDGSFKSFLALRKNGDPDA |
| 35 | KPPRQRVSENSFYKIPGRSGFKVSNGQIYLSFGKIGQTLTSVIPEFQLKRLETAIKLKK |
| | FELCRDERDMAKPGRFWISVAYEIPKPEKVPVVSKQITYLAIGASRLGVVSPKGEFCL |
| | NLPRSDYHWKPQINALQERLEGVVKGSRKWKKRMAACTRMFAKLGHQQKQHGQ |
| | YEVVKKLLRHGVHFVVTELKVRSKPGALADASKSDRKGSPTGPNWSAQNTGNIAR |
| | LIQKLTDKASEHGGTVIKRNPPLLSLEERQLPDAQRKIFIAKKLREEFLADQK |
| SEQ | MAKREKKDDVVLRGTKMRIYPTDRQVTLMDMWRRRCISLWNLLLNLETAAYGAK |
| ID | NTRSKLGWRSIWARVVEENHAKALIVYQHGKCKKDGSFVLKRDGTVKHPPRERFP |
| NO: | GDRKILLGLFDALRHTLDKGAKCKCNVNQPYALTRAWLDETGHGARTADIIAWLK |
| 36 | DFKGECDCTAISTAAKYCPAPPTAELLTKIKRAAPADDLPVDQAILLDLFGALRGGL |
| | KQKECDHTHARTVAYFEKHELAGRAEDILAWLIAHGGTCDCKIVEEAANHCPGPRL |
| | FIWEHELAMIMARLKAEPRTEWIGDLPSHAAQTVVKDLVKALQTMLKERAKAAAG |
| | DESARKTGFPKFKKQAYAAGSVYFPNTTMFFDVAAGRVQLPNGCGSMRCEIPRQLV |
| | AELLERNLKPGLVIGAQLGLLGGRIWRQGDRWYLSCQWERPQPTLLPKTGRTAGVK |
| | IAASIVFTTYDNRGQTKEYPMPPADKKLTAVHLVAGKQNSRALEAQKEKEKKLKAR |
| | KERLRLGKLEKGHDPNALKPLKRPRVRRSKLFYKSAARLAACEAIERDRRDGFLHR |
| | VTNEIVHKFDAVSVQKMSVAPMMRRQKQKEKQIESKKNEAKKEDNGAAKKPRNL |
| | KPVRKLLRHVAMARGRQFLEYKYNDLRGPGSVLIADRLEPEVQECSRCGTKNPQM |
| | KDGRRLLRCIGVLPDGTDCDAVLPRNRNAARNAEKRLRKHREAHNA |
| SEQ | MNEVLPIPAVGEDAADTIMRGSKMRIYPSVRQAATMDLWRRRCIQLWNLLLELEQA |
| ID | AYSGENRRTQIGWRSIWATVVEDSHAEAVRVAREGKKRKDGTFRKAPSGKEIPPLD |
| NO: | PAMLAKIQRQMNGAVDVDPKTGEVTPAQPRLFMWEHELQKIMARLKQAPRTHWID |
| 37 | DLPSHAAQSVVKDLIKALQAMLRERKKRASGIGGRDTGFPKFKKNRYAAGSVYFA |
| | NTQLRFEAKRGKAGDPDAVRGEFARVKLPNGVGWMECRMPRHINAAHAYAQATL |
| | MGGRIWRQGENWYLSCQWKMPKPAPLPRAGRTAAIKIAAAIPITTVDNRGQTREYA |
| | MPPIDRERIAAHAAAGRAQSRALEARKRRAKKREAYAKKRHAKKLERGIAAKPPGR |
| | ARIKLSPGFYAAAAKLAKLEAEDANAREAWLHEITTQIVRNFDVIAVPRMEVAKLM |
| | KKPEPPEEKEEQVKAPWQGKRRSLKAARVMMRRTAMALIQTTLKYKAVDLRGPQ |
| | AYEEIAPLDVTAAACSGCGVLKPEWKMARAKGREIMRCQEPLPGGKTCNTVLTYT |
| | RNSARVIGRELAVRLAERQKA |
| SEQ | MTTQKTYNFCFYDQRFFELSKEAGEVYSRSLEEFWKIYDETGVWLSKFDLQKHMR |
| ID | NKLERKLLHSDSFLGAMQQVHANLASWKQAKKVVPDACPPRKPKFLQAILFKKSQI |
| NO: | KYKNGFLRLTLGTEKEFLYLKWDINIPLPIYGSVTYSKTRGWKINLCLETEVEQKNLS |
| 38 | ENKYLSIDLGVKRVATIFDGENTITLSGKKFMGLMHYRNKLNGKTQSRLSHKKKGS |
| | NNYKKIQRAKRKTTDRLLNIQKEMLHKYSSFIVNYAIRNDIGNIIIGDNSSTHDSPNM |
| | RGKTNQKISQNPEQKLKNYIKYKFESISGRVDIVPEPYTSRKCPHCKNIKKSSPKGRT |
| | YKCKKCGFIFDRDGVGAINIYNENVSFGQIISPGRIRSLTEPIGMKFHNEIYFKSYVAA |
| SEQ | MSVRSFQARVECDKQTMEHLWRTHKVFNERLPEIIKILFKMKRGECGQNDKQKSLY |
| ID | KSISQSILEANAQNADYLLNSVSIKGWKPGTAKKYRNASFTWADDAAKLSSQGIHV |
| NO: | YDKKQVLGDLPGMMSQMVCRQSVEAISGHIELTKKWEKEHNEWLKEKEKWESED |
| 39 | EHKKYLDLREKFEQFEQSIGGKITKRRGRWHLYLKWLSDNPDFAAWRGNKAVINPL |
| | SEKAQIRINKAKPNKKNSVERDEFFKANPEMKALDNLHGYYERNFVRRRKTKKNPD |
| | GFDHKPTFTLPHPTIHPRWFVFNKPKTNPEGYRKLILPKKAGDLGSLEMRLLTGEKN |
| | KGNYPDDWISVKFKADPRLSLIRPVKGRRVVRKGKEQGQTKETDSYEFFDKHLKK |
| | WRPAKLSGVKLIFPDKTPKAAYLYFTCDIPDEPLTETAKKIQWLETGDVTKKGKKR |
| | KKKVLPHGLVSCAVDLSMRRGTTGFATLCRYENGKIHILRSRNLWVGYKEGKGCH |
| | PYRWTEGPDLGHIAKHKREIRILRSKRGKPVKGEESHIDLQKHIDYMGEDRFKKAAR |
| | TIVNFALNTENAASKNGFYPRADVLLLENLEGLIPDAEKERGINRALAGWNRRHLVE |
| | RVIEMAKDAGFKRRVFEIPPYGTSQVCSKCGALGRRYSIIRENNRREIRFGYVEKLFA |
| | CPNCGYCANADHNASVNLNRRFLIEDSFKSYYDWKRLSEKKQKEEIETIESKLMDK |
| | LCAMHKISRGSISK |
| SEQ | MHLWRTHCVFNQRLPALLKRLFAMRRGEVGGNEAQRQVYQRVAQFVLARDAKDS |
| ID | VDLLNAVSLRKRSANSAFKKKATISCNGQAREVTGEEVFAEAVALASKGVFAYDK |
| NO: | DDMRAGLPDSLFQPLTRDAVACMRSHEELVATWKKEYREWRDRKSEWEAEPEHA |
| 40 | LYLNLRPKFEEGEAARGGRFRKRAERDHAYLDWLEANPQLAAWRRKAPPAVVPID |
| | EAGKRRIARAKAWKQASVRAEEFWKRNPELHALHKIHVQYLREFVRPRRTRRNKR |
| | REGFKQRPTFTMPDPVRHPRWCLFNAPQTSPQGYRLLRLPQSRRTVGSVELRLLTGP |
| | SDGAGFPDAWVNVRFKADPRLAQLRPVKVPRTVTRGKNKGAKVEADGFRYYDDQ |
| | LLIERDAQVSGVKLLFRDIRMAPFADKPIEDRLLSATPYLVFAVEIKDEARTERAKAI |
| | RFDETSELTKSGKKRKTLPAGLVSVAVDLDTRGVGFLTRAVIGVPEIQQTHHGVRLL |
| | QSRYVAVGQVEARASGEAEWSPGPDLAHIARHKREIRRLRQLRGKPVKGERSHVRL |
| | QAHIDRMGEDRFKKAARKIVNEALRGSNPAAGDPYTRADVLLYESLETLLPDAERE |
| | RGINRALLRWNRAKLIEHLKRMCDDAGIRHFPVSPFGTSQVCSKCGALGRRYSLAR |
| | ENGRAVIRFGWVERLFACPNPECPGRRPDRPDRPFTCNSDHNASVNLHRVFALGDQ |
| | AVAAFRALAPRDSPARTLAVKRVEDTLRPQLMRVHKLADAGVDSPF |
| SEQ | MATLVYRYGVRAHGSARQQDAVVSDPAMLEQLRLGHELRNALVGVQHRYEDGKR |
| ID | AVWSGFASVAAADHRVTTGETAVAELEKQARAEHSADRTAATRQGTAESLKAAR |
| NO: | AAVKQARADRKAAMAAVAEQAKPKIQALGDDRDAEIKDLYRRFCQDGVLLPRCG |
| 41 | RCAGDLRSDGDCTDCGAAHEPRKLYWATYNAIREDHQTAVKLVEAKRKAGQPAR |
| | LRFRRWTGDGTLTVQLQRMHGPACRCVTCAEKLTRRARKTDPQAPAVAADPAYPP |
| | TDPPRDPALLASGQGKWRNVLQLGTWIPPGEWSAMSRAERRRVGRSHIGWQLGGG |
| | RQLTLPVQLHRQMPADADVAMAQLTRVRVGGRHRMSVALTAKLPDPPQVQGLPP |
| | VALHLGWRQRPDGSLRVATWACPQPLDLPPAVADVVVSHGGRWGEVIMPARWLA |
| | DAEVPPRLLGRRDKAMEPVLEALADWLEAHTEACTARMTPALVRRWRSQGRLAG |
| | LTNRWRGQPPTGSAEILTYLEAWRIQDKLLWERESHLRRRLAARRDDAWRRVASW |
| | LARHAGVLVVDDADIAELRRRDDPADTDPTMPASAAQAARARAALAAPGRLRHLA |
| | TITATRDGLGVHTVASAGLTRLHRKCGHQAQPDPRYAASAVVTCPGCGNGYDQDY |
| | NAAMLMLDRQQQP |
| SEQ | MSRVELHRAYKFRLYPTPAQVAELAEWERQLRRLYNLAHSQRLAAMQRHVRPKSP |
| ID | GVLKSECLSCGAVAVAEIGTDGKAKKTVKHAVGCSVLECRSCGGSPDAEGRTAHT |
| NO: | AACSFVDYYRQGREMTQLLEEDDQLARVVCSARQETLRDLEKAWQRWHKMPGFG |
| 42 | KPHFKKRIDSCRIYFSTPKSWAVDLGYLSFTGVASSVGRIKIRQDRVWPGDAKFSSC |
| | HVVRDVDEWYAVFPLTFTKEIEKPKGGAVGINRGAVHAIADSTGRVVDSPKFYARS |
| | LGVIRHRARLLDRKVPFGRAVKPSPTKYHGLPKADIDAAAARVNASPGRLVYEARA |
| | RGSIAAAEAHLAALVLPAPRQTSQLPSEGRNRERARRFLALAHQRVRRQREWFLHN |
| | ESAHYAQSYTKIAIEDWSTKEMTSSEPRDAEEMKRVTRARNRSILDVGWYELGRQI |
| | AYKSEATGAEFAKVDPGLRETETHVPEAIVRERDVDVSGMLRGEAGISGTCSRCGG |
| | LLRASASGHADAECEVCLHVEVGDVNAAVNVLKRAMFPGAAPPSKEKAKVTIGIK |
| | GRKKKRAA |
| SEQ | MSRVELHRAYKFRLYPTPVQVAELSEWERQLRRLYNLGHEQRLLTLTRHLRPKSPG |
| ID | VLKGECLSCDSTQVQEVGADGRPKTTVRHAEQCPTLACRSCGALRDAEGRTAHTV |
| NO: | ACAFVDYYRQGREMTELLAADDQLARVVCSARQEVLRDLDKAWQRWRKMPGFG |
| 43 | KPRFKRRTDSCRIYFSTPKAWKLEGGHLSFTGAATTVGAIKMRQDRNWPASVQFSS |
| | CHVVRDVDEWYAVFPLTFVAEVARPKGGAVGINRGAVHAIADSTGRVVDSPRYYA |
| | RALGVIRHRARLFDRKVPSGHAVKPSPTKYRGLSAIEVDRVARATGFTPGRVVTEAL |
| | NRGGVAYAECALAAIAVLGHGPERPLTSDGRNREKARKFLALAHQRVRRQREWFL |
| | HNESAHYARTYSKIAIEDWSTKEMTASEPQGEETRRVTRSRNRSILDVGWYELGRQ |
| | LAYKTEATGAEFAQVDPGLKETETNVPKAIADARDVDVSGMLRGEAGISGTCSKCG |
| | GLLRAPASGHADAECEICLNVEVGDVNAAVNVLKRAMFPGDAPPASGEKPKVSIGI |
| | KGRQKKKKAA |
| SEQ | MEAIATGMSPERRVELGILPGSVELKRAYKFRLYPMKVQQAELSEWERQLRRLYNL |
| ID | AHEQRLAALLRYRDWDFQKGACPSCRVAVPGVHTAACDHVDYFRQAREMTQLLE |
| NO: | VDAQLSRVICCARQEVLRDLDKAWQRWRKKLGGRPRFKRRTDSCRIYLSTPKHWEI |
| 44 | AGRYLRLSGLASSVGEIRIEQDRAFPEGALLSSCSIVRDVDEWYACLPLTFTQPIERAP |
| | HRSVGLNRGVVHALADSDGRVVDSPKFFERALATVQKRSRDLARKVSGSRNAHKA |
| | RIKLAKAHQRVRRQRAAFLHQESAYYSKGFDLVALEDMSVRKMTATAGEAPEMGR |
| | GAQRDLNRGILDVGWYELARQIDYKRLAHGGELLRVDPGQTTPLACVTEEQPARGI |
| | SSACAVCGIPLARPASGNARMRCTACGSSQVGDVNAAENVLTRALSSAPSGPKSPK |
| | ASIKIKGRQKRLGTPANRAGEASGGDPPVRGPVEGGTLAYVVEPVSESQSDT |
| SEQ | MTVRTYKYRAYPTPEQAEALTSWLRFASQLYNAALEHRKNAWGRHDAHGRGFRF |
| ID | WDGDAAPRKKSDPPGRWVYRGGGGAHISKNDQGKLLTEFRREHAELLPPGMPALV |
| NO: | QHEVLARLERSMAAFFQRATKGQKAGYPRWRSEHRYDSLTFGLTSPSKERFDPETG |
| 45 | ESLGRGKTVGAGTYHNGDLRLTGLGELRILEHRRIPMGAIPKSVIVRRSGKRWFVSI |
| | AMEMPSVEPAASGRPAVGLDMGVVTWGTAFTADTSAAAALVADLRRMATDPSDC |
| | RRLEELEREAAQLSEVLAHCRARGLDPARPRRCPKELTKLYRRSLHRLGELDRACA |
| | RIRRRLQAAHDIAEPVPDEAGSAVLIEGSNAGMRHARRVARTQRRVARRTRAGHAH |
| | SNRRKKAVQAYARAKERERSARGDHRHKVSRALVRQFEEISVEALDIKQLTVAPEH |
| | NPDPQPDLPAHVQRRRNRGELDAAWGAFFAALDYKAADAGGRVARKPAPHTTQE |
| | CARCGTLVPKPISLRVHRCPACGYTAPRTVNSARNVLQRPLEEPGRAGPSGANGRG |
| | VPHAVA |
| SEQ | MNCRYRYRIYPTPGQRQSLARLFGCVRVVWNDALFLCRQSEKLPKNSELQKLCITQ |
| ID | AKKTEARGWLGQVSAIPLQQSVADLGVAFKNFFQSRSGKRKGKKVNPPRVKRRNN |
| NO: | RQGARFTRGGFKVKTSKVYLARIGDIKIKWSRPLPSEPSSVTVIKDCAGQYFLSFVVE |
| 46 | VKPEIKPPKNPSIGIDLGLKTFASCSNGEKIDSPDYSRLYRKLKRCQRRLAKRQRGSK |
| | RRERMRVKVAKLNAQIRDKRKDFLHKLSTKVVNENQVIALEDLNVGGMLKNRKLS |
| | RAISQAGWYEFRSLCEGKAEKHNRDFRVISRWEPTSQVCSECGYRWGKIDLSVRSIV |
| | CINCGVEHDRDDNASVNIEQAGLKVGVGHTHDSKRTGSACKTSNGAVCVEPSTHRE |
| | YVQLTLFDW |
| SEQ | MKSRWTFRCYPTPEQEQHLARTFGCVRFVWNWALRARTDAFRAGERIGYPATDKA |
| ID | LTLLKQQPETVWLNEVSSVCLQQALRDLQVAFSNFFDKRAAHPSFKRKEARQSANY |
| NO: | TERGFSFDHERRILKLAKIGAIKVKWSRKAIPHPSSIRLIRTASGKYFVSLVVETQPAP |
| 47 | MPETGESVGVDFGVARLATLSNGERISNPKHGAKWQRRLAFYQKRLARATKGSKR |
| | RMRIKRHVARIHEKIGNSRSDTLHKLSTDLVTRFDLICVEDLNLRGMVKNHSLARSL |
| | HDASIGSAIRMIEEKAERYGKNVVKIDRWFPSSKTCSDCGHIVEQLPLNVREWTCPE |
| | CGTTHDRDANAAANILAVGQTVSAHGGTVRRSRAKASERKSQRSANRQGVNRA |
| SEQ | KEPLNIGKTAKAVFKEIDPTSLNRAANYDASIELNCKECKFKPFKNVKRYEFNFYNN |
| ID | WYRCNPNSCLQSTYKAQVRKVEIGYEKLKNEILTQMQYYPWFGRLYQNFFHDERD |
| NO: | KMTSLDEIQVIGVQNKVFFNTVEKAWREIIKKRFKDNKETMETIPELKHAAGHGKR |
| 48 | KLSNKSLLRRRFAFVQKSFKFVDNSDVSYRSFSNNIACVLPSRIGVDLGGVISRNPKR |
| | EYIPQEISFNAFWKQHEGLKKGRNIEIQSVQYKGETVKRIEADTGEDKAWGKNRQR |
| | RFTSLILKLVPKQGGKKVWKYPEKRNEGNYEYFPIPIEFILDSGETSIRFGGDEGEAG |
| | KQKHLVIPFNDSKATPLASQQTLLENSRFNAEVKSCIGLAIYANYFYGYARNYVISSI |
| | YHKNSKNGQAITAIYLESIAHNYVKAIERQLQNLLLNLRDFSFMESHKKELKKYFGG |
| | DLEGTGGAQKRREKEEKIEKEIEQSYLPRLIRLSLTKMVTKQVEM |
| SEQ | ELIVNENKDPLNIGKTAKAVFKEIDPTSINRAANYDASIELACKECKFKPFNNTKRHD |
| ID | FSFYSNWHRCSPNSCLQSTYRAKIRKTEIGYEKLKNEILNQMQYYPWFGRLYQNFFN |
| NO: | DQRDKMTSLDEIQVTGVQNKIFFNTVEKAWREIIKKRFRDNKETMRTIPDLKNKSGH |
| 49 | GSRKLSNKSLLRRRFAFAQKSFKLVDNSDVSYRAFSNNVACVLPSKIGVDIGGIINKD |
| | LKREYIPQEITFNVFWKQHDGLKKGRNIEIHSVQYKGEIVKRIEADTGEDKAWGKNR |
| | QRRFTSLILKITPKQGGKKIWKFPEKKNASDYEYFPIPIEFILDNGDASIKFGGEEGEV |
| | GKQKHLLIPFNDSKATPLSSKQMLLETSRFNAEVKSTIGLALYANYFVSYARNYVIK |
| | STYHKNSKKGQIVTEIYLESISQNFVRAIQRQLQSLMLNLKDWGFMQTHKKELKKY |
| | FGSDLEGSKGGQKRREKEEKIEKEIEASYLPRLIRLSLTKSVTKAEEM |
| SEQ | PEEKTSKLKPNSINLAANYDANEKFNCKECKFHPFKNKKRYEFNFYNNLHGCKSCT |
| ID | KSTNNPAVKRIEIGYQKLKFEIKNQMEAYPWFGRLRINFYSDEKRKMSELNEMQVT |
| NO: | GVKNKIFFDAIECAWREILKKRFRESKETLITIPKLKNKAGHGARKHRNKKLLIRRRA |
| 50 | FMKKNFHFLDNDSISYRSFANNIACVLPSKVGVDIGGIISPDVGKDIKPVDISLNLMW |
| | ASKEGIKSGRKVEIYSTQYDGNMVKKIEAETGEDKSWGKNRKRRQTSLLLSIPKPSK |
| | QVQEFDFKEWPRYKDIEKKVQWRGFPIKIIFDSNHNSIEFGTYQGGKQKVLPIPFNDS |
| | KTTPLGSKMNKLEKLRFNSKIKSRLGSAIAANKFLEAARTYCVDSLYHEVSSANAIG |
| | KGKIFIEYYLEILSQNYIEAAQKQLQRFIESIEQWFVADPFQGRLKQYFKDDLKRAKC |
| | FLCANREVQTTCYAAVKLHKSCAEKVKDKNKELAIKERNNKEDAVIKEVEASNYPR |
| | VIRLKLTKTITNKAM |
| SEQ | SESENKIIEQYYAFLYSFRDKYEKPEFKNRGDIKRKLQNKWEDFLKEQNLKNDKKLS |
| ID | NYIFSNRNFRRSYDREEENEEGIDEKKSKPKRINCFEKEKNLKDQYDKDAINASANK |
| NO: | DGAQKWGCFECIFFPMYKIESGDPNKRIIINKTRFKLFDFYLNLKGCKSCLRSTYHPY |
| 170 | RSNVYIESNYDKLKREIGNFLQQKNIFQRMRKAKVSEGKYLTNLDEYRLSCVAMHF |
| | KNRWLFFDSIQKVLRETIKQRLKQMRESYDEQAKTKRSKGHGRAKYEDQVRMIRR |
| | RAYSAQAHKLLDNGYITLFDYDDKEINKVCLTAINQEGFDIGGYLNSDIDNVMPPIEI |
| | SFHLKWKYNEPILNIESPFSKAKISDYLRKIREDLNLERGKEGKARSKKNVRRKVLAS |
| | KGEDGYKKIFTDFFSKWKEELEGNAMERVLSQSSGDIQWSKKKRIHYTTLVLNINLL |
| | DKKGVGNLKYYEIAEKTKILSFDKNENKFWPITIQVLLDGYEIGTEYDEIKQLNEKTS |
| | KQFTIYDPNTKIIKIPFTDSKAVPLGMLGINIATLKTVKKTERDIKVSKIFKGGLNSKIV |
| | SKIGKGIYAGYFPTVDKEILEEVEEDTLDNEFSSKSQRNIFLKSIIKNYDKMLKEQLFD |
| | FYSFLVRNDLGVRFLTDRELQNIEDESFNLEKRFFETDRDRIARWFDNTNTDDGKEK |
| | FKKLANEIVDSYKPRLIRLPVVRVIKRIQPVKQREM |
| SEQ | KYSTRDFSELNEIQVTACKQDEFFKVIQNAWREIIKKRFLENRENFIEKKIFKNKKGR |
| ID | GKRQESDKTIQRNRASVMKNFQLIENEKIILRAPSGHVACVFPVKVGLDIGGFKTDD |
| NO: | LEKNIFPPRTITINVFWKNRDRQRKGRKLEVWGIKARTKLIEKVHKWDKLEEVKKK |
| 51 | RLKSLEQKQEKSLDNWSEVNNDSFYKVQIDELQEKIDKSLKGRTMNKILDNKAKES |
| | KEAEGLYIEWEKDFEGEMLRRIEASTGGEEKWGKRRQRRHTSLLLDIKNNSRGSKEI |
| | INFYSYAKQGKKEKKIEFFPFPLTITLDAEEESPLNIKSIPIEDKNATSKYFSIPFTETRA |
| | TPLSILGDRVQKFKTKNISGAIKRNLGSSISSCKIVQNAETSAKSILSLPNVKEDNNME |
| | IFINTMSKNYFRAMMKQMESFIFEMEPKTLIDPYKEKAIKWFEVAASSRAKRKLKKL |
| | SKADIKKSELLLSNTEEFEKEKQEKLEALEKEIEEFYLPRIVRLQLTKTILETPVM |
| SEQ | KKLQLLGHKILLKEYDPNAVNAAANFETSTAELCGQCKMKPFKNKRRFQYTFGKN |
| ID | YHGCLSCIQNVYYAKKRIVQIAKEELKHQLTDSIASIPYKYTSLFSNTNSIDELYILKQ |
| NO: | ERAAFFSNTNSIDELYITGIENNIAFKVISAIWDEIIKKRRQRYAESLTDTGTVKANRG |
| 52 | HGGTAYKSNTRQEKIRALQKQTLHMVTNPYISLARYKNNYIVATLPRTIGMHIGAIK |
| | DRDPQKKLSDYAINFNVFWSDDRQLIELSTVQYTGDMVRKIEAETGENNKWGENM |
| | KRTKTSLLLEILTKKTTDELTFKDWAFSTKKEIDSVTKKTYQGFPIGIIFEGNESSVKF |
| | GSQNYFPLPFDAKITPPTAEGFRLDWLRKGSFSSQMKTSYGLAIYSNKVTNAIPAYVI |
| | KNMFYKIARAENGKQIKAKFLKKYLDIAGNNYVPFIIMQHYRVLDTFEEMPISQPKV |
| | IRLSLTKTQHIIIKKDKTDSKM |
| SEQ | NTSNLINLGKKAINISANYDANLEVGCKNCKFLSSNGNFPRQTNVKEGCHSCEKSTY |
| ID | EPSIYLVKIGERKAKYDVLDSLKKFTFQSLKYQSKKSMKSRNKKPKELKEFVIFANK |
| NO: | NKAFDVIQKSYNHLILQIKKEINRMNSKKRKKNHKRRLFRDREKQLNKLRLIESSNL |
| 53 | FLPRENKGNNHVFTYVAIHSVGRDIGVIGSYDEKLNFETELTYQLYFNDDKRLLYAY |
| | KPKQNKIIKIKEKLWNLRKEKEPLDLEYEKPLNKSITFSIKNDNLFKVSKDLMLRRAK |
| | FNIQGKEKLSKEERKINRDLIKIKGLVNSMSYGRFDELKKEKNIWSPHIYREVRQKEI |
| | KPCLIKNGDRIEIFEQLKKKMERLRRFREKRQKKISKDLIFAERIAYNFHTKSIKNTSN |
| | KINIDQEAKRGKASYMRKRIGYETFKNKYCEQCLSKGNVYRNVQKGCSCFENPFD |
| | WIKKGDENLLPKKNEDLRVKGAFRDEALEKQIVKIAFNIAKGYEDFYDNLGESTEK |
| | DLKLKFKVGTTINEQESLKL |
| SEQ | TSNPIKLGKKAINISANYDSNLQIGCKNCKFLSYNGNFPRQTNVKEGCHSCEKSTYEP |
| ID | PVYTVRIGERRSKYDVLDSLKKFIFLSLKYRQSKKMKTRSKGIRGLEEFVISANLKKA |
| NO: | MDVIQKSYRHLILNIKNEIVRMNGKKRNKNHKRLLFRDREKQLNKLRLIEGSSFFKP |
| 54 | PTVKGDNSIFTCVAIHNIGRDIGIAGDYFDKLEPKIELTYQLYYEYNPKKESEINKRLL |
| | YAYKPKQNKIIEIKEKLWNLRKEKSPLDLEYEKPLTKSITFLVKRDGVFRISKDLMLR |
| | KAKFIIQGKEKLSKEERKINRDLIKIKSNIISLTYGRFDELKKDKTIWSPHIFRDVKQG |
| | KITPCIERKGDRMDIFQQLRKKSERLRENRKKRQKKISKDLIFAERIAYNFHTKSIKNT |
| | SNLINIKHEAKRGKASYMRKRIGNETFRIKYCEQCFPKNNVYKNVQKGCSCFEDPFE |
| | YIKKGNEDLIPNKNQDLKAKGAFRDDALEKQIIKVAFNIAKGYEDFYENLKKTTEKD |
| | IRLKFKVGTIISEEM |
| SEQ | NNSINLSKKAINISANYDANLQVRCKNCKFLSSNGNFPRQTDVKEGCHSCEKSTYEP |
| ID | PVYDVKIGEIKAKYEVLDSLKKFTFQSLKYQLSKSMKFRSKKIKELKEFVIFAKESKA |
| NO: | LNVINRSYKHLILNIKNDINRMNSKKRIKNHKGRLFLDRQKQLSKLKLIEGSSFFVPA |
| 55 | KNVGNKSVFTCVAIHSIGRDIGIAGLYDSFTKPVNEITYQIFFSGERRLLYAYKPKQL |
| | KILSIKENLWSLKNEKKPLDLLYEKPLGKNLNFNVKGGDLFRVSKDLMIRNAKFNV |
| | HGRQRLSDEERLINRNFIKIKGEVVSLSYGRFEELKKDRKLWSPHIFKDVRQNKIKPC |
| | LVMQGQRIDIFEQLKRKLELLKKIRKSRQKKLSKDLIFGERIAYNFHTKSIKNTSNKIN |
| | IDSDAKRGRASYMRKRIGNETFKLKYCDVCFPKANVYRRVQNGCSCSENPYNYIKK |
| | GDKDLLPKKDEGLAIKGAFRDEKLNKQIIKVAFNIAKGYEDFYDDLKKRTEKDVDL |
| | KFKIGTTVLDQKPMEIFDGIVITWL |
| SEQ | LLTTVVETNNLAKKAINVAANFDANIDRQYYRCTPNLCRFIAQSPRETKEKDAGCSS |
| ID | CTQSTYDPKVYVIKIGKLLAKYEILKSLKRFLFMNRYFKQKKTERAQQKQKIGTELN |
| NO: | EMSIFAKATNAMEVIKRATKHCTYDIIPETKSLQMLKRRRHRVKVRSLLKILKERRM |
| 56 | KIKKIPNTFIEIPKQAKKNKSDYYVAAALKSCGIDVGLCGAYEKNAEVEAEYTYQLY |
| | YEYKGNSSTKRILYCYNNPQKNIREFWEAFYIQGSKSHVNTPGTIRLKMEKFLSPITIE |
| | SEALDFRVWNSDLKIRNGQYGFIKKRSLGKEAREIKKGMGDIKRKIGNLTYGKSPSE |
| | LKSIHVYRTERENPKKPRAARKKEDNFMEIFEMQRKKDYEVNKKRRKEATDAAKI |
| | MDFAEEPIRHYHTNNLKAVRRIDMNEQVERKKTSVFLKRIMQNGYRGNYCRKCIKA |
| | PEGSNRDENVLEKNEGCLDCIGSEFIWKKSSKEKKGLWHTNRLLRRIRLQCFTTAKA |
| | YENFYNDLFEKKESSLDIIKLKVSITTKSM |
| SEQ | ASTMNLAKQAINFAANYDSNLEIGCKGCKFMSTWSKKSNPKFYPRQNNQANKCHS |
| ID | CTYSTGEPEVPIIEIGERAAKYKIFTALKKFVFMSVAYKERRRQRFKSKKPKELKELA |
| NO: | ICSNREKAMEVIQKSVVHCYGDVKQEIPRIRKIKVLKNHKGRLFYKQKRSKIKIAKLE |
| 57 | KGSFFKTFIPKVHNNGCHSCHEASLNKPILVTTALNTIGADIGLINDYSTIAPTETDIS |
| | WQVYYEFIPNGDSEAVKKRLLYFYKPKGALIKSIRDKYFKKGHENAVNTGFFKYQG |
| | KIVKGPIKFVNNELDFARKPDLKSMKIKRAGFAIPSAKRLSKEDREINRESIKIKNKIY |
| | SLSYGRKKTLSDKDIIKHLYRPVRQKGVKPLEYRKAPDGFLEFFYSLKRKERRLRKQ |
| | KEKRQKDMSEIIDAADEFAWHRHTGSIKKTTNHINFKSEVKRGKVPIMKKRIANDSF |
| | NTRHCGKCVKQGNAINKYYIEKQKNCFDCNSIEFKWEKAALEKKGAFKLNKRLQYI |
| | VKACFNVAKAYESFYEDFRKGEEESLDLKFKIGTTTTLKQYPQNKARAM |
| SEQ | HSHNLMLTKLGKQAINFAANYDANLEIGCKNCKFLSYSPKQANPKKYPRQTDVHED |
| ID | GNIACHSCMQSTKEPPVYIVPIGERKSKYEILTSLNKFTFLALKYKEKKRQAFRAKKP |
| NO: | KELQELAIAFNKEKAIKVIDKSIQHLILNIKPEIARIQRQKRLKNRKGKLLYLHKRYAI |
| 58 | KMGLIKNGKYFKVGSPKKDGKKLLVLCALNTIGRDIGIIGNIEENNRSETEITYQLYF |
| | DCLDANPNELRIKEIEYNRLKSYERKIKRLVYAYKPKQTKILEIRSKFFSKGHENKVN |
| | TGSFNFENPLNKSISIKVKNSAFDFKIGAPFIMLRNGKFHIPTKKRLSKEEREINRTLSK |
| | IKGRVFRLTYGRNISEQGSKSLHIYRKERQHPKLSLEIRKQPDSFIDEFEKLRLKQNFIS |
| | KLKKQRQKKLADLLQFADRIAYNYHTSSLEKTSNFINYKPEVKRGRTSYIKKRIGNE |
| | GFEKLYCETCIKSNDKENAYAVEKEELCFVCKAKPFTWKKTNKDKLGIFKYPSRIKD |
| | FIRAAFTVAKSYNDFYENLKKKDLKNEIFLKFKIGLILSHEKKNHISIAKSVAEDERIS |
| | GKSIKNILNKSIKLEKNCYSCFFHKEDM |
| SEQ | SLERVIDKRNLAKKAINIAANFDANINKGFYRCETNQCMFIAQKPRKTNNTGCSSCL |
| ID | QSTYDPVIYVVKVGEMLAKYEILKSLKRFVFMNRSFKQKKTEKAKQKERIGGELNE |
| NO: | MSIFANAALAMGVIKRAIRHCHVDIRPEINRLSELKKTKHRVAAKSLVKIVKQRKTK |
| 59 | WKGIPNSFIQIPQKARNKDADFYVASALKSGGIDIGLCGTYDKKPHADPRWTYQLYF |
| | DTEDESEKRLLYCYNDPQAKIRDFWKTFYERGNPSMVNSPGTIEFRMEGFFEKMTPI |
| | SIESKDFDFRVWNKDLLIRRGLYEIKKRKNLNRKAREIKKAMGSVKRVLANMTYGK |
| | SPTDKKSIPVYRVEREKPKKPRAVRKEENELADKLENYRREDFLIRNRRKREATEIA |
| | KIIDAAEPPIRHYHTNHLRAVKRIDLSKPVARKNTSVFLKRIMQNGYRGNYCKKCIK |
| | GNIDPNKDECRLEDIKKCICCEGTQNIWAKKEKLYTGRINVLNKRIKQMKLECFNVA |
| | KAYENFYDNLAALKEGDLKVLKLKVSIPALNPEASDPEEDM |
| SEQ | NASINLGKRAINLSANYDSNLVIGCKNCKFLSFNGNFPRQTNVREGCHSCDKSTYAP |
| ID | EVYIVKIGERKAKYDVLDSLKKFTFQSLKYQIKKSMRERSKKPKELLEFVIFANKDK |
| NO: | AFNVIQKSYEHLILNIKQEINRMNGKKRIKNHKKRLFKDREKQLNKLRLIGSSSLFFP |
| 60 | RENKGDKDLFTYVAIHSVGRDIGVAGSYESHIEPISDLTYQLFINNEKRLLYAYKPKQ |
| | NKIIELKENLWNLKKEKKPLDLEFTKPLEKSITFSVKNDKLFKVSKDLMLRQAKFNI |
| | QGKEKLSKEERQINRDFSKIKSNVISLSYGRFEELKKEKNIWSPHIYREVKQKEIKPCI |
| | VRKGDRIELFEQLKRKMDKLKKFRKERQKKISKDLNFAERIAYNFHTKSIKNTSNKI |
| | NIDQEAKRGKASYMRKRIGNESFRKKYCEQCFSVGNVYHNVQNGCSCFDNPIELIK |
| | KGDEGLIPKGKEDRKYKGALRDDNLQMQIIRVAFNIAKGYEDFYNNLKEKTEKDLK |
| | LKFKIGTTISTQESNNKEM |
| SEQ | SNLIKLGKQAINFAANYDANLEVGCKNCKFLSSTNKYPRQTNVHLDNKMACRSCN |
| ID | QSTMEPAIYIVRIGEKKAKYDIYNSLTKFNFQSLKYKAKRSQRFKPKQPKELQELSIA |
| NO: | VRKEKALDIIQKSIDHLIQDIRPEIPRIKQQKRYKNHVGKLFYLQKRRKNKLNLIGKG |
| 61 | SFFKVFSPKEKKNELLVICALTNIGRDIGLIGNYNTIINPLFEVTYQLYYDYIPKKNNK |
| | NVQRRLLYAYKSKNEKILKLKEAFFKRGHENAVNLGSFSYEKPLEKSLTLKIKNDKD |
| | DFQVSPSLRIRTGRFFVPSKRNLSRQEREINRRLVKIKSKIKNMTYGKFETARDKQSV |
| | HIFRLERQKEKLPLQFRKDEKEFMEEFQKLKRRTNSLKKLRKSRQKKLADLLQLSEK |
| | VVYNNHTGTLKKTSNFLNFSSSVKRGKTAYIKELLGQEGFETLYCSNCINKGQKTRY |
| | NIETKEKCFSCKDVPFVWKKKSTDKDRKGAFLFPAKLKDVIKATFTVAKAYEDFYD |
| | NLKSIDEKKPYIKFKIGLILAHVRHEHKARAKEEAGQKNIYNKPIKIDKNCKECFFFK |
| | EEAM |
| SEQ | NTTRKKFRKRTGFPQSDNIKLAYCSAIVRAANLDADIQKKHNQCNPNLCVGIKSNEQ |
| ID | SRKYEHSDRQALLCYACNQSTGAPKVDYIQIGEIGAKYKILQMVNAYDFLSLAYNL |
| NO: | TKLRNGKSRGHQRMSQLDEVVIVADYEKATEVIKRSINHLLDDIRGQLSKLKKRTQ |
| 62 | NEHITEHKQSKIRRKLRKLSRLLKRRRWKWGTIPNPYLKNWVFTKKDPELVTVALL |
| | HKLGRDIGLVNRSKRRSKQKLLPKVGFQLYYKWESPSLNNIKKSKAKKLPKRLLIPY |
| | KNVKLFDNKQKLENAIKSLLESYQKTIKVEFDQFFQNRTEEIIAEEQQTLERGLLKQL |
| | EKKKNEFASQKKALKEEKKKIKEPRKAKLLMEESRSLGFLMANVSYALFNTTIEDL |
| | YKKSNVVSGCIPQEPVVVFPADIQNKGSLAKILFAPKDGFRIKFSGQHLTIRTAKFKIR |
| | GKEIKILTKTKREILKNIEKLRRVWYREQHYKLKLFGKEVSAKPRFLDKRKTSIERRD |
| | PNKLADQTDDRQAELRNKEYELRHKQHKMAERLDNIDTNAQNLQTLSFWVGEAD |
| | KPPKLDEKDARGFGVRTCISAWKWFMEDLLKKQEEDPLLKLKLSIM |
| SEQ | PKKPKFQKRTGFPQPDNLRKEYCLAIVRAANLDADFEKKCTKCEGIKTNKKGNIVK |
| ID | GRTYNSADKDNLLCYACNISTGAPAVDYVFVGALEAKYKILQMVKAYDFHSLAYN |
| NO: | LAKLWKGRGRGHQRMGGLNEVVIVSNNEKALDVIEKSLNHFHDEIRGELSRLKAKF |
| 63 | QNEHLHVHKESKLRRKLRKISRLLKRRRWKWDVIPNSYLRNFTFTKTRPDFISVALL |
| | HRVGRDIGLVTKTKIPKPTDLLPQFGFQIYYTWDEPKLNKLKKSRLRSEPKRLLVPY |
| | KKIELYKNKSVLEEAIRHLAEVYTEDLTICFKDFFETQKRKFVSKEKESLKRELLKEL |
| | TKLKKDFSERKTALKRDRKEIKEPKKAKLLMEESRSLGFLAANTSYALFNLIAADLY |
| | TKSKKACSTKLPRQLSTILPLEIKEHKSTTSLAIKPEEGFKIRFSNTHLSIRTPKFKMKG |
| | ADIKALTKRKREILKNATKLEKSWYGLKHYKLKLYGKEVAAKPRFLDKRNPSIDRR |
| | DPKELMEQIENRRNEVKDLEYEIRKGQHQMAKRLDNVDTNAQNLQTKSFWVGEAD |
| | KPPELDSMEAKKLGLRTCISAWKWFMKDLVLLQEKSPNLKLKLSLTEM |
| SEQ | KFSKRQEGFLIPDNIDLYKCLAIVRSANLDADVQGHKSCYGVKKNGTYRVKQNGKK |
| ID | GVKEKGRKYVFDLIAFKGNIEKIPHEAIEEKDQGRVIVLGKFNYKLILNIEKNHNDRA |
| NO: | SLEIKNKIKKLVQISSLETGEFLSDLLSGKIGIDEVYGIIEPDVFSGKELVCKACQQSTY |
| 64 | APLVEYMPVGELDAKYKILSAIKGYDFLSLAYNLSRNRANKKRGHQKLGGGELSEV |
| | VISANYDKALNVIKRSINHYHVEIKPEISKLKKKMQNEPLKVMKQARIRRELHQLSR |
| | KVKRLKWKWGMIPNPELQNIIFEKKEKDFVSYALLHTLGRDIGLFKDTSMLQVPNIS |
| | DYGFQIYYSWEDPKLNSIKKIKDLPKRLLIPYKRLDFYIDTILVAKVIKNLIELYRKSY |
| | VYETFGEEYGYAKKAEDILFDWDSINLSEGIEQKIQKIKDEFSDLLYEARESKRQNFV |
| | ESFENILGLYDKNFASDRNSYQEKIQSMIIKKQQENIEQKLKREFKEVIERGFEGMDQ |
| | NKKYYKVLSPNIKGGLLYTDTNNLGFFRSHLAFMLLSKISDDLYRKNNLVSKGGNK |
| | GILDQTPETMLTLEFGKSNLPNISIKRKFFNIKYNSSWIGIRKPKFSIKGAVIREITKKV |
| | RDEQRLIKSLEGVWHKSTHFKRWGKPRFNLPRHPDREKNNDDNLMESITSRREQIQL |
| | LLREKQKQQEKMAGRLDKIDKEIQNLQTANFQIKQIDKKPALTEKSEGKQSVRNALS |
| | AWKWFMEDLIKYQKRTPILQLKLAKM |
| SEQ | KFSKRQEGFVIPENIGLYKCLAIVRSANLDADVQGHVSCYGVKKNGTYVLKQNGKK |
| ID | SIREKGRKYASDLVAFKGDIEKIPFEVIEEKKKEQSIVLGKFNYKLVLDVMKGEKDR |
| NO: | ASLTMKNKSKKLVQVSSLGTDEFLLTLLNEKFGIEEIYGIIEPEVFSGKKLVCKACQQ |
| 65 | STYAPLVEYMPVGELDSKYKILSAIKGYDFLSLAYNLARHRSNKKRGHQKLGGGEL |
| | SEVVISANNAKALNVIKRSLNHYYSEIKPEISKLRKKMQNEPLKVGKQARMRRELH |
| | QLSRKVKRLKWKWGKIPNLELQNITFKESDRDFISYALLHTLGRDIGMFNKTEIKMP |
| | SNILGYGFQIYYDWEEPKLNTIKKSKNTPKRILIPYKKLDFYNDSILVARAIKELVGLF |
| | QESYEWEIFGNEYNYAKEAEVELIKLDEESINGNVEKKLQRIKENFSNLLEKAREKK |
| | RQNFIESFESIARLYDESFTADRNEYQREIQSFIIEKQKQSIEKKLKNEFKKIVEKKFNE |
| | QEQGKKHYRVLNPTIINEFLPKDKNNLGFLRSKIAFILLSKISDDLYKKSNAVSKGGE |
| | KGIIKQQPETILDLEFSKSKLPSINIKKKLFNIKYTSSWLGIRKPKFNIKGAKIREITRRV |
| | RDVQRTLKSAESSWYASTHFRRWGFPRFNQPRHPDKEKKSDDRLIESITLLREQIQIL |
| | LREKQKGQKEMAGRLDDVDKKIQNLQTANFQIKQTGDKPALTEKSAGKQSFRNAL |
| | SAWKWFMENLLKYQNKTPDLKLKIARTVM |
| SEQ | KWIEPNNIDFNKCLAITRSANLDADVQGHKMCYGIKTNGTYKAIGKINKKHNTGIIE |
| ID | KRRTYVYDLIVTKEKNEKIVKKTDFMAIDEEIEFDEKKEKLLKKYIKAEVLGTGELIR |
| NO: | KDLNDGEKFDDLCSIEEPQAFRRSELVCKACNQSTYASDIRYIPIGEIEAKYKILKAIK |
| 66 | GYDFLSLKYNLGRLRDSKKRGHQKMGQGELKEFVICANKEKALDVIKRSLNHYLN |
| | EVKDEISRLNKKMQNEPLKVNDQARWRRELNQISRRLKRLKWKWGEIPNPELKNLI |
| | FKSSRPEFVSYALIHTLGRDIGLINETELKPNNIQEYGFQIYYKWEDPELNHIKKVKNI |
| | PKRFIIPYKNLDLFGKYTILSRAIEGILKLYSSSFQYKSFKDPNLFAKEGEKKITNEDFE |
| | LGYDEKIKKIKDDFKSYKKALLEKKKNTLEDSLNSILSVYEQSLLTEQINNVKKWKE |
| | GLLKSKESIHKQKKIENIEDIISRIEELKNVEGWIRTKERDIVNKEETNLKREIKKELKD |
| | SYYEEVRKDFSDLKKGEESEKKPFREEPKPIVIKDYIKFDVLPGENSALGFFLSHLSFN |
| | LFDSIQYELFEKSRLSSSKHPQIPETILDL |
| SEQ | FRKFVKRSGAPQPDNLNKYKCIAIVRAANLDADIMSNESSNCVMCKGIKMNKRKTA |
| ID | KGAAKTTELGRVYAGQSGNLLCTACTKSTMGPLVDYVPIGRIRAKYTILRAVKEYD |
| NO: | FLSLAYNLARTRVSKKGGRQKMHSLSELVIAAEYEIAWNIIKSSVIHYHQETKEEISG |
| 67 | LRKKLQAEHIHKNKEARIRREMHQISRRIKRLKWKWHMIPNSELHNFLFKQQDPSFV |
| | AVALLHTLGRDIGMINKPKGSAKREFIPEYGFQIYYKWMNPKLNDINKQKYRKMPK |
| | RSLIPYKNLNVFGDRELIENAMHKLLKLYDENLEVKGSKFFKTRVVAISSKESEKLK |
| | RDLLWKGELAKIKKDFNADKNKMQELFKEVKEPKKANALMKQSRNMGFLLQNISY |
| | GALGLLANRMYEASAKQSKGDATKQPSIVIPLEMEFGNAFPKLLLRSGKFAMNVSS |
| | PWLTIRKPKFVIKGNKIKNITKLMKDEKAKLKRLETSYHRATHFRPTLRGSIDWDSP |
| | YFSSPKQPNTHRRSPDRLSADITEYRGRLKSVEAELREGQRAMAKKLDSVDMTASN |
| | LQTSNFQLEKGEDPRLTEIDEKGRSIRNCISSWKKFMEDLMKAQEANPVIKIKIALKD |
| | ESSVLSEDSM |
| SEQ | KFHPENLNKSYCLAIVRAANLDADIQGHINCIGIKSNKSDRNYENKLESLQNVELLC |
| ID | KACTKSTYKPNINSVPVGEKKAKYSILSEIKKYDFNSLVYNLKKYRKGKSRGHQKL |
| NO: | NELRELVITSEYKKALDVINKSVNHYLVNIKNKMSKLKKILQNEHIHVGTLARIRRE |
| 68 | RNRISRKLDHYRKKWKFVPNKILKNYVFKNQSPDFVSVALLHKLGRDIGLITKTAIL |
| | QKSFPEYSLQLYYKYDTPKLNYLKKSKFKSLPKRILISYKYPKFDINSNYIEESIDKLL |
| | KLYEESPIYKNNSKIIEFFKKSEDNLIKSENDSLKRGIMKEFEKVTKNFSSKKKKLKEE |
| | LKLKNEDKNSKMLAKVSRPIGFLKAYLSYMLFNIISNRIFEFSRKSSGRIPQLPSCHNL |
| | GNQFENFKNELQDSNIGSKKNYKYFCNLLLKSSGFNISYEEEHLSIKTPNFFINGRKL |
| | KEITSEKKKIRKENEQLIKQWKKLTFFKPSNLNGKKTSDKIRFKSPNNPDIERKSEDNI |
| | VENIAKVKYKLEDLLSEQRKEFNKLAKKHDGVDVEAQCLQTKSFWIDSNSPIKKSLE |
| | KKNEKVSVKKKMKAIRSCISAWKWFMADLIEAQKETPMIKLKLALM |
| SEQ | TTLVPSHLAGIEVMDETTSRNEDMIQKETSRSNEDENYLGVKNKCGINVHKSGRGSS |
| ID | KHEPNMPPEKSGEGQMPKQDSTEMQQRFDESVTGETQVSAGATASIKTDARANSGP |
| NO: | RVGTARALIVKASNLDRDIKLGCKPCEYIRSELPMGKKNGCNHCEKSSDIASVPKVE |
| 69 | SGFRKAKYELVRRFESFAADSISRHLGKEQARTRGKRGKKDKKEQMGKVNLDEIAI |
| | LKNESLIEYTENQILDARSNRIKEWLRSLRLRLRTRNKGLKKSKSIRRQLITLRRDYR |
| | KWIKPNPYRPDEDPNENSLRLHTKLGVDIGVQGGDNKRMNSDDYETSFSITWRDTA |
| | TRKICFTKPKGLLPRHMKFKLRGYPELILYNEELRIQDSQKFPLVDWERIPIFKLRGVS |
| | LGKKKVKALNRITEAPRLVVAKRIQVNIESKKKKVLTRYVYNDKSINGRLVKAEDS |
| | NKDPLLEFKKQAEEINSDAKYYENQEIAKNYLWGCEGLHKNLLEEQTKNPYLAFKY |
| | GFLNIV |
| SEQ | LDFKRTCSQELVLLPEIEGLKLSGTQGVTSLAKKLINKAANVDRDESYGCHHCIHTR |
| ID | TSLSKPVKKDCNSCNQSTNHPAVPITLKGYKIAFYELWHRFTSWAVDSISKALHRNK |
| NO: | VMGKVNLDEYAVVDNSHIVCYAVRKCYEKRQRSVRLHKRAYRCRAKHYNKSQPK |
| 70 | VGRIYKKSKRRNARNLKKEAKRYFQPNEITNGSSDALFYKIGVDLGIAKGTPETEVK |
| | VDVSICFQVYYGDARRVLRVRKMDELQSFHLDYTGKLKLKGIGNKDTFTIAKRNES |
| | LKWGSTKYEVSRAHKKFKPFGKKGSVKRKCNDYFRSIASWSCEAASQRAQSNLKN |
| | AFPYQKALVKCYKNLDYKGVKKNDMWYRLCSNRIFRYSRIAEDIAQYQSDKGKAK |
| | FEFVILAQSVAEYDISAIM |
| SEQ | VFLTDDKRKTALRKIRSAFRKTAEIALVRAQEADSLDRQAKKLTIETVSFGAPGAKN |
| ID | AFIGSLQGYNWNSHRANVPSSGSAKDVFRITELGLGIPQSAHEASIGKSFELVGNVVR |
| NO: | YTANLLSKGYKKGAVNKGAKQQREIKGKEQLSFDLISNGPISGDKLINGQKDALAW |
| 71 | WLIDKMGFHIGLAMEPLSSPNTYGITLQAFWKRHTAPRRYSRGVIRQWQLPFGRQL |
| | APLIHNFFRKKGASIPIVLTNASKKLAGKGVLLEQTALVDPKKWWQVKEQVTGPLS |
| | NIWERSVPLVLYTATFTHKHGAAHKRPLTLKVIRISSGSVFLLPLSKVTPGKLVRAW |
| | MPDINILRDGRPDEAAYKGPDLIRARERSFPLAYTCVTQIADEWQKRALESNRDSITP |
| | LEAKLVTGSDLLQIHSTVQQAVEQGIGGRISSPIQELLAKDALQLVLQQLFMTVDLL |
| | RIQWQLKQEVADGNTSEKAVGWAIRISNIHKDAYKTAIEPCTSALKQAWNPLSGFEE |
| | RTFQLDASIVRKRSTAKTPDDELVIVLRQQAAEMTVAVTQSVSKELMELAVRHSAT |
| | LHLLVGEVASKQLSRSADKDRGAMDHWKLLSQSM |
| SEQ | EDLLQKALNTATNVAAIERHSCISCLFTESEIDVKYKTPDKIGQNTAGCQSCTFRVGY |
| ID | SGNSHTLPMGNRIALDKLRETIQRYAWHSLLFNVPPAPTSKRVRAISELRVAAGRER |
| NO: | LFTVITFVQTNILSKLQKRYAANWTPKSQERLSRLREEGQHILSLLESGSWQQKEVV |
| 72 | REDQDLIVCSALTKPGLSIGAFCRPKYLKPAKHALVLRLIFVEQWPGQIWGQSKRTR |
| | RMRRRKDVERVYDISVQAWALKGKETRISECIDTMRRHQQAYIGVLPFLILSGSTVR |
| | GKGDCPILKEITRMRYCPNNEGLIPLGIFYRGSANKLLRVVKGSSFTLPMWQNIETLP |
| | HPEPFSPEGWTATGALYEKNLAYWSALNEAVDWYTGQILSSGLQYPNQNEFLARLQ |
| | NVIDSIPRKWFRPQGLKNLKPNGQEDIVPNEFVIPQNAIRAHHVIEWYHKTNDLVAK |
| | TLLGWGSQTTLNQTRPQGDLRFTYTRYYFREKEVPEV |
| SEQ | VPKKKLMRELAKKAVFEAIFNDPIPGSFGCKRCTLIDGARVTDAIEKKQGAKRCAGC |
| ID | EPCTFHTLYDSVKHALPAATGCDRTAIDTGLWEILTALRSYNWMSFRRNAVSDASQ |
| NO: | KQVWSIEELAIWADKERALRVILSALTHTIGKLKNGFSRDGVWKGGKQLYENLAQK |
| 73 | DLAKGLFANGEIFGKELVEADHDMLAWTIVPNHQFHIGLIRGNWKPAAVEASTAFD |
| | ARWLTNGAPLRDTRTHGHRGRRFNRTEKLTVLCIKRDGGVSEEFRQERDYELSVML |
| | LQPKNKLKPEPKGELNSFEDLHDHWWFLKGDEATALVGLTSDPTVGDFIQLGLYIR |
| | NPIKAHGETKRRLLICFEPPIKLPLRRAFPSEAFKTWEPTINVFRNGRRDTEAYYDIDR |
| | ARVFEFPETRVSLEHLSKQWEVLRLEPDRENTDPYEAQQNEGAELQVYSLLQEAAQ |
| | KMAPKVVIDPFGQFPLELFSTFVAQLFNAPLSDTKAKIGKPLDSGFVVESHLHLLEED |
| | FAYRDFVRVTFMGTEPTFRVIHYSNGEGYWKKTVLKGKNNIRTALIPEGAKAAVDA |
| | YKNKRCPLTLEAAILNEEKDRRLVLGNKALSLLAQTARGNLTILEALAAEVLRPLSG |
| | TEGVVHLHACVTRHSTLTESTETDNM |
| SEQ | VEKLFSERLKRAMWLKNEAGRAPPAETLTLKHKRVSGGHEKVKEELQRVLRSLSGT |
| ID | NQAAWNLGLSGGREPKSSDALKGEKSRVVLETVVFHSGHNRVLYDVIEREDQVHQ |
| NO: | RSSIMHMRRKGSNLLRLWGRSGKVRRKMREEVAEIKPVWHKDSRWLAIVEEGRQS |
| 74 | VVGISSAGLAVFAVQESQCTTAEPKPLEYVVSIWFRGSKALNPQDRYLEFKKLKTTE |
| | ALRGQQYDPIPFSLKRGAGCSLAIRGEGIKFGSRGPIKQFFGSDRSRPSHADYDGKRR |
| | LSLFSKYAGDLADLTEEQWNRTVSAFAEDEVRRATLANIQDFLSISHEKYAERLKKR |
| | IESIEEPVSASKLEAYLSAIFETFVQQREALASNFLMRLVESVALLISLEEKSPRVEFRV |
| | ARYLAESKEGFNRKAM |
| SEQ | VVITQSELYKERLLRVMEIKNDRGRKEPRESQGLVLRFTQVTGGQEKVKQKLWLIFE |
| ID | GFSGTNQASWNFGQPAGGRKPNSGDALKGPKSRVTYETVVFHFGLRLLSAVIERHN |
| NO: | LKQQRQTMAYMKRRAAARKKWARSGKKCSRMRNEVEKIKPKWHKDPRWFDIVK |
| 75 | EGEPSIVGISSAGFAIYIVEEPNFPRQDPLEIEYAISIWFRRDRSQYLTFKKIQKAEKLK |
| | ELQYNPIPFRLKQEKTSLVFESGDIKFGSRGSIEHFRDEARGKPPKADMDNNRRLTMF |
| | SVFSGNLTNLTEEQYARPVSGLLAPDEKRMPTLLKKLQDFFTPIHEKYGERIKQRLA |
| | NSEASKRPFKKLEEYLPAIYLEFRARREGLASNWVLVLINSVRTLVRIKSEDPYIEFK |
| | VSQYLLEKEDNKAL |
| SEQ | KQDALFEERLKKAIFIKRQADPLQREELSLLPPNRKIVTGGHESAKDTLKQILRAING |
| ID | TNQASWNPGTPSGKRDSKSADALAGPKSRVKLETVVFHVGHRLLKKVVEYQGHQK |
| NO: | QQHGLKAFMRTCAAMRKKWKRSGKVVGELREQLANIQPKWHYDSRPLNLCFEGK |
| 76 | PSVVGLRSAGIALYTIQKSVVPVKEPKPIEYAVSIWFRGPKAMDREDRCLEFKKLKIA |
| | TELRKLQFEPIVSTLTQGIKGFSLYIQGNSVKFGSRGPIKYFSNESVRQRPPKADPDGN |
| | KRLALFSKFSGDLSDLTEEQWNRPILAFEGIIRRATLGNIQDYLTVGHEQFAISLEQLL |
| | SEKESVLQMSIEQQRLKKNLGKKAENEWVESFGAEQARKKAQGIREYISGFFQEYCS |
| | QREQWAENWVQQLNKSVRLFLTIQDSTPFIEFRVARYLPKGEKKKGKAM |
| SEQ | ANHAERHKRLRKEANRAANRNRPLVADCDTGDPLVGICRLLRRGDKMQPNKTGCR |
| ID | SCEQVEPELRDAILVSGPGRLDNYKYELFQRGRAMAVHRLLKRVPKLNRPKKAAG |
| NO: | NDEKKAENKKSEIQKEKQKQRRMMPAVSMKQVSVADFKHVIENTVRHLFGDRRDR |
| 77 | EIAECAALRAASKYFLKSRRVRPRKLPKLANPDHGKELKGLRLREKRAKLKKEKEK |
| | QAELARSNQKGAVLHVATLKKDAPPMPYEKTQGRNDYTTFVISAAIKVGATRGTKP |
| | LLTPQPREWQCSLYWRDGQRWIRGGLLGLQAGIVLGPKLNRELLEAVLQRPIECRM |
| | SGCGNPLQVRGAAVDFFMTTNPFYVSGAAYAQKKFKPFGTKRASEDGAAAKAREK |
| | LMTQLAKVLDKVVTQAAHSPLDGIWETRPEAKLRAMIMALEHEWIFLRPGPCHNA |
| | AEEVIKCDCTGGHAILWALIDEARGALEHKEFYAVTRAHTHDCEKQKLGGRLAGFL |
| | DLLIAQDVPLDDAPAARKIKTLLEATPPAPCYKAATSIATCDCEGKFDKLWAIIDATR |
| | AGHGTEDLWARTLAYPQNVNCKCKAGKDLTHRLADFLGLLIKRDGPFRERPPHKV |
| | TGDRKLVFSGDKKCKGHQYVILAKAHNEEVVRAWISRWGLKSRTNKAGYAATELN |
| | LLLNWLSICRRRWMDMLTVQRDTPYIRMKTGRLVVDDKKERKAM |
| SEQ | AKQREALRVALERGIVRASNRTYTLVTNCTKGGPLPEQCRMIERGKARAMKWEPK |
| ID | LVGCGSCAAATVDLPAIEEYAQPGRLDVAKYKLTTQILAMATRRMMVRAAKLSRR |
| NO: | KGQWPAKVQEEKEEPPEPKKMLKAVEMRPVAIVDFNRVIQTTIEHLWAERANADE |
| 78 | AELKALKAAAAYFGPSLKIRARGPPKAAIGRELKKAHRKKAYAERKKARRKRAEL |
| | ARSQARGAAAHAAIRERDIPPMAYERTQGRNDVTTIPIAAAIKIAATRGARPLPAPKP |
| | MKWQCSLYWNEGQRWIRGGMLTAQAYAHAANIHRPMRCEMWGVGNPLKVRAFE |
| | GRVADPDGAKGRKAEFRLQTNAFYVSGAAYRNKKFKPFGTDRGGIGSARKKRERL |
| | MAQLAKILDKVVSQAAHSPLDDIWHTRPAQKLRAMIKQLEHEWMFLRPQAPTVEG |
| | TKPDVDVAGNMQRQIKALMAPDLPPIEKGSPAKRFTGDKRKKGERAVRVAEAHSD |
| | EVVTAWISRWGIQTRRNEGSYAAQELELLLNWLQICRRRWLDMTAAQRVSPYIRM |
| | KSGRMITDAADEGVAPIPLVENM |
| SEQ | KSISGRSIKHMACLKDMLKSEITEIEEKQKKESLRKWDYYSKFSDEILFRRNLNVSAN |
| ID | HDANACYGCNPCAFLKEVYGFRIERRNNERIISYRRGLAGCKSCVQSTGYPPIEFVRR |
| NO: | KFGADKAMEIVREVLHRRNWGALARNIGREKEADPILGELNELLLVDARPYFGNKS |
| 79 | AANETNLAFNVITRAAKKFRDEGMYDIHKQLDIHSEEGKVPKGRKSRLIRIERKHKA |
| | IHGLDPGETWRYPHCGKGEKYGVWLNRSRLIHIKGNEYRCLTAFGTTGRRMSLDVA |
| | CSVLGHPLVKKKRKKGKKTVDGTELWQIKKATETLPEDPIDCTFYLYAAKPTKDPFI |
| | LKVGSLKAPRWKKLHKDFFEYSDTEKTQGQEKGKRVVRRGKVPRILSLRPDAKFKV |
| | SIWDDPYNGKNKEGTLLRMELSGLDGAKKPLILKRYGEPNTKPKNFVFWRPHITPHP |
| | LTFTPKHDFGDPNKKTKRRRVFNREYYGHLNDLAKMEPNAKFFEDREVSNKKNPK |
| | AKNIRIQAKESLPNIVAKNGRWAAFDPNDSLWKLYLHWRGRRKTIKGGISQEFQEF |
| | KERLDLYKKHEDESEWKEKEKLWENHEKEWKKTLEIHGSIAEVSQRCVMQSMMGP |
| | LDGLVQKKDYVHIGQSSLKAADDAWTFSANRYKKATGPKWGKISVSNLLYDANQ |
| | ANAELISQSISKYLSKQKDNQGCEGRKMKFLIKIIEPLRENFVKHTRWLHEMTQKDC |
| | EVRAQFSRVSM |
| SEQ | FPSDVGADALKHVRMLQPRLTDEVRKVALTRAPSDRPALARFAAVAQDGLAFVRH |
| ID | LNVSANHDSNCTFPRDPRDPRRGPCEPNPCAFLREVWGFRIVARGNERALSYRRGL |
| NO: | AGCKSCVQSTGFPSVPFHRIGADDCMRKLHEILKARNWRLLARNIGREREADPLLTE |
| 80 | LSEYLLVDARTYPDGAAPNSGRLAENVIKRAAKKFRDEGMRDIHAQLRVHSREGKV |
| | PKGRLQRLRRIERKHRAIHALDPGPSWEAEGSARAEVQGVAVYRSQLLRVGHHTQQ |
| | IEPVGIVARTLFGVGRTDLDVAVSVLGAPLTKRKKGSKTLESTEDFRIAKARETRAE |
| | DKIEVAFVLYPTASLLRDEIPKDAFPAMRIDRFLLKVGSVQADREILLQDDYYRFGD |
| | AEVKAGKNKGRTVTRPVKVPRLQALRPDAKFRVNVWADPFGAGDSPGTLLRLEVS |
| | GVTRRSQPLRLLRYGQPSTQPANFLCWRPHRVPDPMTFTPRQKFGERRKNRRTRRP |
| | RVFERLYQVHIKHLAHLEPNRKWFEEARVSAQKWAKARAIRRKGAEDIPVVAPPAK |
| | RRWAALQPNAELWDLYAHDREARKRFRGGRAAEGEEFKPRLNLYLAHEPEAEWES |
| | KRDRWERYEKKWTAVLEEHSRMCAVADRTLPQFLSDPLGARMDDKDYAFVGKSA |
| | LAVAEAFVEEGTVERAQGNCSITAKKKFASNASRKRLSVANLLDVSDKADRALVFQ |
| | AVRQYVQRQAENGGVEGRRMAFLRKLLAPLRQNFVCHTRWLHM |
| SEQ | AARKKKRGKIGITVKAKEKSPPAAGPFMARKLVNVAANVDGVEVHLCVECEADAH |
| ID | GSASARLLGGCRSCTGSIGAEGRLMGSVDVDRERVIAEPVHTETERLGPDVKAFEAG |
| NO: | TAESKYAIQRGLEYWGVDLISRNRARTVRKMEEADRPESSTMEKTSWDEIAIKTYSQ |
| 81 | AYHASENHLFWERQRRVRQHALALFRRARERNRGESPLQSTQRPAPLVLAALHAEA |
| | AAISGRARAEYVLRGPSANVRAAAADIDAKPLGHYKTPSPKVARGFPVKRDLLRAR |
| | HRIVGLSRAYFKPSDVVRGTSDAIAHVAGRNIGVAGGKPKEIEKTFTLPFVAYWEDV |
| | DRVVHCSSFKADGPWVRDQRIKIRGVSSAVGTFSLYGLDVAWSKPTSFYIRCSDIRK |
| | KFHPKGFGPMKHWRQWAKELDRLTEQRASCVVRALQDDEELLQTMERGQRYYDV |
| | FSCAATHATRGEADPSGGCSRCELVSCGVAHKVTKKAKGDTGIEAVAVAGCSLCES |
| | KLVGPSKPRVHRQMAALRQSHALNYLRRLQREWEALEAVQAPTPYLRFKYARHLE |
| | VRSM |
| SEQ | AAKKKKQRGKIGISVKPKEGSAPPADGPFMARKLVNVAANVDGVEVNLCIECEAD |
| ID | AHGSAPARLLGGCKSCTGSIGAEGRLMGSVDVDRADAIAKPVNTETEKLGPDVQAF |
| NO: | EAGTAETKYALQRGLEYWGVDLISRNRSRTVRRTEEGQPESATMEKTSWDEIAIKS |
| 82 | YTRAYHASENHLFWERQRRVRQHALALFKRAKERNRGDSTLPREPGHGLVAIAAL |
| | ACEAYAVGGRNLAETVVRGPTFGTARAVRDVEIASLGRYKTPSPKVAHGSPVKRDF |
| | LRARHRIVGLARAYYRPSDVVRGTSDAIAHVAGRNIGVAGGKPRAVEAVFTLPFVA |
| | YWEDVDRVVHCSSFQVSAPWNRDQRMKIAGVTTAAGTFSLHGGELKWAKPTSFYI |
| | RCSDTRRKFRPKGFGPMKRWRQWAKDLDRLVEQRASCVVRALQDDAALLETMER |
| | GQRYYDVFACAVTHATRGEADRLAGCSRCALTPCQEAHRVTTKPRGDAGVEQVQT |
| | SDCSLCEGKLVGPSKPRLHRTLTLLRQEHGLNYLRRLQREWESLEAVQVPTPYLRFK |
| | YARHLEVRSM |
| SEQ | TDSQSESVPEVVYALTGGEVPGRVPPDGGSAEGARNAPTGLRKQRGKIKISAKPSKP |
| ID | GSPASSLARTLVNEAANVDGVQSSGCATCRMRANGSAPRALPIGCVACASSIGRAP |
| NO: | QEETVCALPTTQGPDVRLLEGGHALRKYDIQRALEYWGVDLIGRNLDRQAGRGME |
| 83 | PAEGATATMKRVSMDELAVLDFGKSYYASEQHLFAARQRRVRQHAKALKIRAKHA |
| | NRSGSVKRALDRSRKQVTALAREFFKPSDVVRGDSDALAHVVGRNLGVSRHPAREI |
| | PQTFTLPLCAYWEDVDRVISCSSLLAGEPFARDQEIRIEGVSSALGSLRLYRGAIEWH |
| | KPTSLYIRCSDTRRKFRPRGGLKKRWRQWAKDLDRLVEQRACCIVRSLQADVELLQ |
| | TMERAQRFYDVHDCAATHVGPVAVRCSPCAGKQFDWDRYRLLAALRQEHALNYL |
| | RRLQREWESLEAQQVKMPYLRFKYARKLEVSGPLIGLEVRREPSMGTAIAEM |
| SEQ | AGTAGRRHGSLGARRSINIAGVTDRHGRWGCESCVYTRDQAGNRARCAPCDQSTY |
| ID | APDVQEVTIGQRQAKYTIFLTLQSFSWTNTMRNNKRAAAGRSKRTTGKRIGQLAEIK |
| NO: | ITGVGLAHAHNVIQRSLQHNITKMWRAEKGKSKRVARLKKAKQLTKRRAYFRRRM |
| 84 | SRQSRGNGFFRTGKGGIHAVAPVKIGLDVGMIASGSSEPADEQTVTLDAIWKGRKK |
| | KIRLIGAKGELAVAACRFREQQTKGDKCIPLILQDGEVRWNQNNWQCHPKKLVPLC |
| | GLEVSRKFVSQADRLAQNKVASPLAARFDKTSVKGTLVESDFAAVLVNVTSIYQQC |
| | HAMLLRSQEPTPSLRVQRTITSM |
| SEQ | GVRFSPAQSQVFFRTVIPQSVEARFAINMAAIHDAAGAFGCSVCRFEDRTPRNAKAV |
| ID | HGCSPCTRSTNRPDVFVLPVGAIKAKYDVFMRLLGFNWTHLNRRQAKRVTVRDRIG |
| NO: | QLDELAISMLTGKAKAVLKKSICHNVDKSFKAMRGSLKKLHRKASKTGKSQLRAK |
| 85 | LSDLRERTNTTQEGSHVEGDSDVALNKIGLDVGLVGKPDYPSEESVEVVVCLYFVG |
| | KVLILDAQGRIRDMRAKQYDGFKIPIIQRGQLTVLSVKDLGKWSLVRQDYVLAGDL |
| | RFEPKISKDRKYAECVKRIALITLQASLGFKERIPYYVTKQVEIKNASHIAFVTEAIQN |
| | CAENFREMTEYLMKYQEKSPDLKVLLTQLM |
| SEQ | RAVVGKVFLEQARRALNLATNFGTNHRTGCNGCYVTPGKLSIPQDGEKNAAGCTS |
| ID | CLMKATASYVSYPKPLGEKVAKYSTLDALKGFPWYSLRLNLRPNYRGKPINGVQEV |
| NO: | APVSKFRLAEEVIQAVQRYHFTELEQSFPGGRRRLRELRAFYTKEYRRAPEQRQHVV |
| 86 | NGDRNIVVVTVLHELGFSVGMFNEVELLPKTPIECAVNVFIRGNRVLLEVRKPQFDK |
| | ERLLVESLWKKDSRRHTAKWTPPNNEGRIFTAEGWKDFQLPLLLGSTSRSLRAIEKE |
| | GFVQLAPGRDPDYNNTIDEQHSGRPFLPLYLYLQGTISQEYCVFAGTWVIPFQDGISP |
| | YSTKDTFQPDLKRKAYSLLLDAVKHRLGNKVASGLQYGRFPAIEELKRLVRMHGAT |
| | RKIPRGEKDLLKKGDPDTPEWWLLEQYPEFWRLCDAAAKRVSQNVGLLLSLKKQP |
| | LWQRRWLESRTRNEPLDNLPLSMALTLHLTNEEAL |
| SEQ | AAVYSKFYIENHFKMGIPETLSRIRGPSIIQGFSVNENYINIAGVGDRDFIFGCKKCKY |
| ID | TRGKPSSKKINKCHPCKRSTYPEPVIDVRGSISEFKYKIYNKLKQEPNQSIKQNTKGR |
| NO: | MNPSDHTSSNDGIIINGIDNRIAYNVIFSSYKHLMEKQINLLRDTTKRKARQIKKYNN |
| 87 | SGKKKHSLRSQTKGNLKNRYHMLGMFKKGSLTITNEGDFITAVRKVGLDISLYKNE |
| | SLNKQEVETELCLNIKWGRTKSYTVSGYIPLPINIDWKLYLFEKETGLTLRLFGNKYK |
| | IQSKKFLIAQLFKPKRPPCADPVVKKAQKWSALNAHVQQMAGLFSDSHLLKRELKN |
| | RMHKQLDFKSLWVGTEDYIKWFEELSRSYVEGAEKSLEFFRQDYFCFNYTKQTTM |
| SEQ | PQQQRDLMLMAANYDQDYGNGCGPCTVVASAAYRPDPQAQHGCKRHLRTLGASA |
| ID | VTHVGLGDRTATITALHRLRGPAALAARARAAQAASAPMTPDTDAPDDRRRLEAID |
| NO: | ADDVVLVGAHRALWSAVRRWADDRRAALRRRLHSEREWLLKDQIRWAELYTLIE |
| 88 | ASGTPPQGRWRNTLGALRGQSRWRRVLAPTMRATCAETHAELWDALAELVPEMA |
| | KDRRGLLRPPVEADALWRAPMIVEGWRGGHSVVVDAVAPPLDLPQPCAWTAVRLS |
| | GDPRQRWGLHLAVPPLGQVQPPDPLKATLAVSMRHRGGVRVRTLQAMAVDADAP |
| | MQRHLQVPLTLQRGGGLQWGIHSRGVRRREARSMASWEGPPIWTGLQLVNRWKG |
| | QGSALLAPDRPPDTPPYAPDAAVAPAQPDTKRARRTLKEACTVCRCAPGHMRQLQ |
| | VTLTGDGTWRRFRLRAPQGAKRKAEVLKVATQHDERIANYTAWYLKRPEHAAGC |
| | DTCDGDSRLDGACRGCRPLLVGDQCFRRYLDKIEADRDDGLAQIKPKAQEAVAAM |
| | AAKRDARAQKVAARAAKLSEATGQRTAATRDASHEARAQKELEAVATEGTTVRH |
| | DAAAVSAFGSWVARKGDEYRHQVGVLANRLEHGLRLQELMAPDSVVADQQRASG |
| | HARVGYRYVLTAM |
| SEQ | AVAHPVGRGNAGSPGARGPEELPRQLVNRASNVTRPATYGCAPCRHVRLSIPKPVL |
| ID | TGCRACEQTTHPAPKRAVRGGADAAKYDLAAFFAGWAADLEGRNRRRQVHAPLD |
| NO: | PQPDPNHEPAVTLQKIDLAEVSIEEFQRVLARSVKHRHDGRASREREKARAYAQVA |
| 89 | KKRRNSHAHGARTRRAVRRQTRAVRRAHRMGANSGEILVASGAEDPVPEAIDHAA |
| | QLRRRIRACARDLEGLRHLSRRYLKTLEKPCRRPRAPDLGRARCHALVESLQAAERE |
| | LEELRRCDSPDTAMRRLDAVLAAAASTDATFATGWTVVGMDLGVAPRGSAAPEVS |
| | PMEMAISVFWRKGSRRVIVSKPIAGMPIRRHELIRLEGLGTLRLDGNHYTGAGVTKG |
| | RGLSEGTEPDFREKSPSTLGFTLSDYRHESRWRPYGAKQGKTARQFFAAMSRELRA |
| | LVEHQVLAPMGPPLLEAHERRFETLLKGQDNKSIHAGGGGRYVWRGPPDSKKRPA |
| | ADGDWFRFGRGHADHRGWANKRHELAANYLQSAFRLWSTLAEAQEPTPYARYKY |
| | TRVTM |
| SEQ | WDFLTLQVYERHTSPEVCVAGNSTKCASGTRKSDHTHGVGVKLGAQEINVSANDD |
| ID | RDHEVGCNICVISRVSLDIKGWRYGCESCVQSTPEWRSIVRFDRNHKEAKGECLSRF |
| NO: | EYWGAQSIARSLKRNKLMGGVNLDELAIVQNENVVKTSLKHLFDKRKDRIQANLK |
| 90 | AVKVRMRERRKSGRQRKALRRQCRKLKRYLRSYDPSDIKEGNSCSAFTKLGLDIGIS |
| | PNKPPKIEPKVEVVFSLFYQGACDKIVTVSSPESPLPRSWKIKIDGIRALYVKSTKVKF |
| | GGRTFRAGQRNNRRKVRPPNVKKGKRKGSRSQFFNKFAVGLDAVSQQLPIASVQGL |
| | WGRAETKKAQTICLKQLESNKPLKESQRCLFLADNWVVRVCGFLRALSQRQGPTPY |
| | IRYRYRCNM |
| SEQ | ARNVGQRNASRQSKRESAKARSRRVTGGHASVTQGVALINAAANADRDHTTGCEP |
| ID | CTWERVNLPLQEVIHGCDSCTKSSPFWRDIKVVNKGYREAKEEIMRIASGISADHLS |
| NO: | RALSHNKVMGRLNLDEVCILDFRTVLDTSLKHLTDSRSNGIKEHIRAVHRKIRMRRK |
| 91 | SGKTARALRKQYFALRRQWKAGHKPNSIREGNSLTALRAVGFDVGVSEGTEPMPAP |
| | QTEVVLSVFYKGSATRILRISSPHPIAKRSWKVKIAGIKALKLIRREHDFSFGRETYNA |
| | SQRAEKRKFSPHAARKDFFNSFAVQLDRLAQQLCVSSVENLWVTEPQQKLLTLAKD |
| | TAPYGIREGARFADTRARLAWNWVFRVCGFTRALHQEQEPTPYCRFTWRSKM |

In some embodiments, a programmable nuclease may be a programmable nickase (e.g., a Cas14a, a Cas14b, or a Cas14e). The present disclosure provides compositions of programmable nickases, capable of introducing a break in a single strand of a double stranded DNA (dsDNA) (“nicking”). In some embodiments the programmable nickase is a programmable DNA nickase. Said programmable nickases include a nickase coupled to a guide nucleic acid that targets a particular region of interest in the dsDNA. In some embodiments, two programmable nickases are combined and delivered together to generate two strand breaks. For example, a first programmable nickase can be targeted to and nicks a first region of dsDNA and a second programmable nickase can be targeted to and nicks a second region of the same dsDNA on the opposing strand. When combined and delivered together to generate nicks on opposing strands of the dsDNA, two strand breaks in the dsDNA can be generated. The strand breaks can be repaired and rejoined by non-homologous end joining (NHEJ) or homology directed repair (HDR). Thus, two programmable nickases disclosed herein can be combined to selectively edit nucleic acid sequences. This can be useful in any genome editing used for therapeutic applications to treat a disease or disorder, or for agricultural applications.

In some embodiments, a programmable nickase can be a Cas protein capable of nicking a single strand of a dsDNA. Cas proteins consistent with the programmable nickases disclosed herein includes Cas14, which is also referred to herein as CasZ. In particular embodiments, a Cas protein consistent with the programable nickases disclosed herein includes Cas14e, which is also referred to herein as CasZe. Cas14e programmable nickases disclosed herein can be used for genome editing purposes to generate strand breaks in order to excise a region of DNA or to subsequently introduce a region of DNA.

A method of nicking a target nucleic acid may comprise contacting the target nucleic acid with a first guide nucleic acid (e.g., a guide nucleic acid comprising a first region that binds to a first programmable nickase having a length of no more than 900 amino acids) and a second guide nucleic acid (e.g., a guide nucleic acid comprising a first region that binds to a second programmable nickase having a length of no more than 900 amino acids). The first guide nucleic acid may comprise a second region that binds to the target nucleic acid, and the second guide nucleic acid may comprise a second region that binds to the target nucleic acid. The second region of the first guide nucleic acid and the second region of the second guide nucleic acid may bind opposing strands of the target nucleic acid.

In some embodiments, the programmable nickases disclosed herein (e.g., Cas14e) can be used in DNA Endonuclease Targeted CRISPR TransReporter (DETECTR) assays. A DETECTR assay utilizes the trans-cleavage abilities of some programmable nucleases and programmable nickases (e.g., CRISPR-Cas effector proteins) to achieve fast and high-fidelity detection of a target DNA in a sample. Following DNA extraction from a biological sample, crRNA that is complementary to the target DNA sequence of interest can bind to the target DNA, initiating indiscriminate ssDNase activity by the programmable nuclease or programmable nickase (e.g., a programmable nickase such as Cas14e). In some embodiments, the extracted DNA is amplified by PCR or isothermal amplification reactions before contacting the DNA to the programmable nickase complexed with a guide RNA. Upon hybridization with the target DNA, the trans-cleavage activity of the programmable nickase is activated, which can then cleave an ssDNA fluorescence-quenching (FQ) reporter molecule. Cleavage of the reporter molecule can provide a fluorescent readout indicating the presence of the target DNA in the sample. In some embodiments, the programmable nickases disclosed herein (e.g., Cas14e) can be combined, or multiplexed, with other programmable nucleases or other programmable nickases in a DETECTR assay.

The programmable nickases of the present disclosure can show enhanced activity, as measured by enhanced cleavage of an ssDNA-FQ reporter, under certain conditions in the presence of the target DNA. For example, the programmable nickases of the present disclosure can have variable levels of activity based on a buffer formulation, a pH level, temperature, or salt. Buffers consistent with the present disclosure include phosphate buffers, Tris buffers, and HEPES buffers. Programmable nickases of the present disclosure (e.g., Cas14e) can show optimal activity in phosphate buffers, Tris buffers, and HEPES buffers. Programmable nickases can also exhibit varying levels of cleavage at different pH levels. For example, enhanced cleavage can be observed between pH 7 and pH 9. In some embodiments, programmable nickases of the present disclosure exhibit enhanced cleavage at about pH 7, about pH 7.1, about pH 7.2, about pH 7.3, about pH 7.4, about pH 7.5, about pH 7.6, about pH 7.7, about pH 7.8, about pH 7.9, about pH 8, about pH 8.1, about pH 8.2, about pH 8.3, about pH 8.4, about pH 8.5, about pH 8.6, about pH 8.7, about pH 8.8, about pH 8.9, about pH 9, from pH 7 to 7.5, from pH 7.5 to 8, from pH 8 to 8.5, from pH 8.5 to 9, or from pH 7 to 8.5.

In some embodiments, the programmable nickases (e.g., Cas14e) of the present disclosure exhibits enhanced cleavage of ssDNA-FQ reporters DNA at a temperature of 25° C. to 50° C. in the presence of target DNA. For example, the programmable nickases (e.g., Cas14e) of the present disclosure can exhibit enhanced cleavage of an ssDNA-FQ reporter at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., from 30° C. to 40° C., from 35° C. to 45° C., or from 35° C. to 40° C.

The programmable nickases (e.g., Cas14e) of the present disclosure may not be sensitive to salt concentrations in a sample in the presence of the target DNA. Advantageously, said programmable nickases can be active and capable of cleaving ssDNA-FQ-reporter sequences under varying salt concentrations from 25 nM salt to 200 mM salt. Various salts are consistent with this property of the programmable nickases disclosed herein, including NaCl or KCl. The programmable nickases of the present disclosure can be active at salt concentrations of from 25 nM to 500 nM salt, from 500 nM to 1000 nM salt, from 1000 nM to 2000 nM salt, from 2000 nM to 3000 nM salt, from 3000 nM to 4000 nM salt, from 4000 nM to 5000 nM salt, from 5000 nM to 6000 nM salt, from 6000 nM to 7000 nM salt, from 7000 nM to 8000 nM salt, from 8000 nM to 9000 nM salt, from 9000 nM to 0.01 mM salt, from 0.01 mM to 0.05 mM salt, from 0.05 mM to 0.1 mM salt, from 0.1 mM to 10 mM salt, from 10 mM to 100 mM salt, or from 100 mM to 500 mM salt. Thus, the programmable nickases (e.g., Cas14e) of the present disclosure can exhibit cleavage activity independent of the salt concentration in a sample.

Programmable nickases (e.g., Cas14e) of the present disclosure can be capable of cleaving any ssDNA-FQ reporter, regardless of its sequence. The programmable nickases provided herein can, thus, be capable of cleaving a universal ssDNA FQ reporter. In some embodiments, the programmable nickases provided herein cleave homopolymer ssDNA-FQ reporter comprising 5 to 20 adenine, 5 to 20 thymines, 5 to 20 cytosines, or 5 to 20 guanines. Programmable nickases of the present disclosure, thus, are capable of cleaving ssDNA-FQ reporters also cleaved by programmable nucleases, as disclosed elsewhere herein, allowing for facile multiplexing of multiple programmable nickases and programmable nucleases in a single assay having a single ssDNA-FQ reporter.

Programmable nickases (e.g., Cas14e) of the present disclosure can bind a wild type protospacer adjacent motif (PAM) or a mutant PAM in a target DNA. In some embodiments, the programmable nickases of the present disclosure are PAM-insensitive and can bind to a target DNA irrespective of the PAM sequence in the target DNA. In some embodiments, the programmable nickases of the present disclosure are PAM-independent and can bind to a target DNA irrespective of the presence of a PAM sequence in the target DNA.

In some embodiments, the programmable nickases of the present disclosure (e.g., a Cas14) is especially useful for genome editing and use in a DETECTR assay due to its small size. The smaller nature of these proteins allows for them to be more easily packaged and delivered with higher efficiency in the context of genome editing and more readily incorporated as a reagent in an assay. In some embodiments, the programmable nickases of the present disclosure are from 400 to 800 amino acid residues long, from 400 to 420 amino acid residues long, from 420 to 440 amino acid residues long, from 440 to 460 amino acid residues long, from 460 to 480 amino acid residues long, from 480 to 500 amino acid residues long, from 500 to 520 amino acid residues long, from 520 to 540 amino acid residues long, from 540 to 560 amino acid residues long, from 560 to 580 amino acid residues long, from 580 to 600 amino acid residues long, from 600 to 620 amino acid residues long, from 620 to 640 amino acid residues long, from 640 to 660 amino acid residues long, from 660 to 680 amino acid residues long, from 680 to 700 amino acid residues long, from 700 to 720 amino acid residues long, from 720 to 740 amino acid residues long, from 740 to 760 amino acid residues long, from 760 to 780 amino acid residues long, from 780 to 800 amino acid residues long, from 400 to 500 amino acid residues long, from 500 to 600 amino acid residues long, from 600 to 700 amino acid residues long, from 700 to 800 amino acid residues long, from 450 to 550 amino acid residues long, from 550 to 650 amino acid residues long, from 650 to 750 amino acid residues long, or from 750 to 800 amino acid residues long. In some embodiments, the programmable nickases of the present disclosure are from 350 to 900 amino acid residues long. In some embodiments, the programmable nickases of the present disclosure are from 500 to 550 amino acid residues long. In preferred embodiments, the programmable Cas14 nickases of the present disclosure are from 480 to 550 amino acids in length.

The programmable nickases (e.g., a Cas14a, Cas14b, or Cas14e programmable nickase) and other reagents (e.g., a guide nucleic acid) can be formulated in a buffer disclosed herein. A wide variety of buffered solutions are compatible with the methods, compositions, reagents, enzymes, and kits disclosed herein. Buffers are compatible with different programmable nickases described herein. Any of the methods, compositions, reagents, enzymes, or kits disclosed herein may comprise a buffer. These buffers may be compatible with the other reagents, samples, and support mediums as described herein for detection of an ailment, such as a disease, cancer, or genetic disorder, or genetic information, such as for phenotyping, genotyping, or determining ancestry. A buffer, as described herein, can enhance the cis- or trans-cleavage rates of any of the programmable nickase described herein. The buffer can increase the discrimination of the programmable nickase for the target nucleic acid. The methods as described herein can be performed in the buffer.

In some embodiments, a buffer may comprise one or more of a buffering agent, a salt, a crowding agent, or a detergent, or any combination thereof. A buffer may comprise a reducing agent. A buffer may comprise a competitor. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. A buffering agent may be compatible with a programmable nickase. A buffer compatible with a programmable nickase may comprise a buffering agent at a concentration of from 1 mM to 200 mM. A buffer compatible with a programmable nickase may comprise a buffering agent at a concentration of from 10 mM to 30 mM. A buffer compatible with a programmable nickase may comprise a buffering agent at a concentration of about 20 mM. A composition (e.g., a composition comprising a programmable nickase) may have a pH of from 2.5 to 3.5. A composition (e.g., a composition comprising a programmable nickase) may have a pH of from 3 to 4. A composition (e.g., a composition comprising a programmable nickase) may have a pH of from 3.5 to 4.5. A composition (e.g., a composition comprising a programmable nickase) may have a pH of from 4 to 5. A composition (e.g., a composition comprising a programmable nickase) may have a pH of from 4.5 to 5.5. A composition (e.g., a composition comprising a programmable nickase) may have a pH of from 5 to 6. A composition (e.g., a composition comprising a programmable nickase) may have a pH of from 5.5 to 6.5. A composition (e.g., a composition comprising a programmable nickase) may have a pH of from 6 to 7. A composition (e.g., a composition comprising a programmable nickase) may have a pH of from 6.5 to 7.5. A composition (e.g., a composition comprising a programmable nickase) may have a pH of from 7 to 8. A composition (e.g., a composition comprising a programmable nickase) may have a pH of from 7.5 to 8.5. A composition (e.g., a composition comprising a programmable nickase) may have a pH of from 8 to 9. A composition (e.g., a composition comprising a programmable nickase) may have a pH of from 8.5 to 9.5. A composition (e.g., a composition comprising a programmable nickase) may have a pH of from 9 to 10. A composition (e.g., a composition comprising a programmable nickase) may have a pH of from 9.5 to 10.5.

A buffer may comprise a salt. Exemplary salts include NaCl, KCl, magnesium acetate, potassium acetate, CaCl₂ and MgCl₂. A buffer may comprise potassium acetate, magnesium acetate, sodium chloride, magnesium chloride, or any combination thereof. A buffer compatible with a programmable nickase may comprise a salt at a concentration of from 5 mM to 100 mM. A buffer compatible with a programmable nickase may comprise a salt at a concentration of from 5 mM to 10 mM. In some embodiments, a buffer compatible with a programmable nickase comprises a salt from 1 mM to 60 mM. In some embodiments, a buffer compatible with a programmable nickase comprises a salt from 1 mM to 10 mM. In some embodiments, a buffer compatible with a programmable nickase comprises a salt at about 105 mM. In some embodiments, a buffer compatible with a programmable nickase comprises a salt at about 55 mM. In some embodiments, a buffer compatible with a programmable nickase comprises a salt at about 7 mM. In some embodiments, a buffer compatible with a programmable nickase comprises a salt, wherein the salt comprises potassium acetate and magnesium acetate. In some embodiments, a buffer compatible with a programmable nickase comprises a salt, wherein the salt comprises sodium chloride and magnesium chloride. In some embodiments, a buffer compatible with a programmable nickase comprises a salt, wherein the salt comprises potassium chloride and magnesium chloride.

A buffer may comprise a crowding agent. Exemplary crowding agents include glycerol and bovine serum albumin. A buffer may comprise glycerol. A crowding agent may reduce the volume of solvent available for other molecules in the solution, thereby increasing the effective concentrations of said molecules. A buffer compatible with a programmable nickase may comprise a crowding agent at a concentration of from 0.01% (v/v) to 10% (v/v). A buffer compatible with a programmable nickase may comprise a crowding agent at a concentration of from 0.5% (v/v) to 10% (v/v).

A buffer may comprise a detergent. Exemplary detergents include Tween, Triton-X, and IGEPAL. A buffer may comprise Tween, Triton-X, or any combination thereof. A buffer compatible with a programmable nickase may comprise Triton-X. A buffer compatible with a programmable nickase may comprise IGEPAL CA-630. In some embodiments, a buffer compatible with a programmable nickase comprises a detergent at a concentration of 2% (v/v) or less. A buffer compatible with a programmable nickase may comprise a detergent at a concentration of 2% (v/v) or less. A buffer compatible with a programmable nickase may comprise a detergent at a concentration of from 0.00001% (v/v) to 0.01% (v/v). A buffer compatible with a programmable nickase may comprise a detergent at a concentration of about 0.01% (v/v).

A buffer may comprise a reducing agent. Exemplary reducing agents comprise dithiothreitol (DTT), ß-mercaptoethanol (BME), or tris(2-carboxyethyl)phosphine (TCEP). A buffer compatible with a programmable nickase may comprise DTT. A buffer compatible with a programmable nickase may comprise a reducing agent at a concentration of from 0.01 mM to 100 mM. A buffer compatible with a programmable nickase may comprise a reducing agent at a concentration of from 0.1 mM to 10 mM. A buffer compatible with a programmable nickase may comprise a reducing agent at a concentration of from 0.5 mM to 2 mM. A buffer compatible with a programmable nickase may comprise a reducing agent at a concentration of from 0.01 mM to 100 mM. A buffer compatible with a programmable nickase may comprise a reducing agent at a concentration of from 0.1 mM to 10 mM. A buffer compatible with a programmable nickase may comprise a reducing agent at a concentration of about 1 mM.

A buffer compatible with a programmable nickase may comprise a competitor. Exemplary competitors compete with the target nucleic acid or the reporter nucleic acid for cleavage by the programmable nickase. Exemplary competitors include heparin, and imidazole, and salmon sperm DNA. A buffer compatible with a programmable nickase may comprise a competitor at a concentration of from 1 μg/mL to 100 μg/mL. A buffer compatible with a programmable nickase may comprise a competitor at a concentration of from 40 μg/mL to 60 μg/mL.

### Guide Nucleic Acids

The reagents of this disclosure may comprise a guide nucleic acid. The guide nucleic acid can bind to a single stranded target nucleic acid or portion thereof as described herein. For example, the guide nucleic acid can bind to a target nucleic acid such as nucleic acid from a virus or a bacterium or other agents responsible for a disease, or an amplicon thereof, as described herein. The guide nucleic acid can bind to a target nucleic acid such as a nucleic acid from a bacterium, a virus, a parasite, a protozoa, a fungus or other agents responsible for a disease, or an amplicon thereof, as described herein and further comprising a mutation, such as a single nucleotide polymorphism (SNP), which can confer resistance to a treatment, such as antibiotic treatment. The guide nucleic acid can bind to a target nucleic acid such as a nucleic acid, preferably DNA, from a cancer gene or gene associated with a genetic disorder, or an amplicon thereof, as described herein. The guide nucleic acid comprises a segment of nucleic acids that are reverse complementary to the target nucleic acid. Often the guide nucleic acid binds specifically to the target nucleic acid. The target nucleic acid may be a reversed transcribed RNA, DNA, DNA amplicon, or synthetic nucleic acids. The target nucleic acid can be a single-stranded DNA or DNA amplicon of a nucleic acid of interest. A guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized.

A guide nucleic acid (gRNA) sequence may hybridize to a target sequence of a target nucleic acid. In some embodiments, a gRNA is a gRNA system (e.g., comprising a crRNA and a tracrRNA or a crRNA and a trancRNA). A crRNA may comprise a repeat region that hybridizes to a region of a tracrRNA. The tracrRNA may bind to a programmable nuclease (e.g., a programmable nickase of the present disclosure). In some embodiments, the repeat region may comprise mutations or truncations with respect to the repeat sequences in pre-crRNA. The repeat sequence of the crRNA may interact with tracrRNA, which may interact with the programmable nuclease (e.g., a programmable nickase), allowing for the crRNA, the tracrRNA and the programmable nuclease to form a complex. This complex may be referred to as a nucleoprotein. The crRNA may comprise a spacer sequence. The spacer sequence may hybridize to a target sequence of the target nucleic acid, where the target sequence is a segment of a target nucleic acid. The spacer sequences may be reverse complementary to the target sequence. In some cases, the spacer sequence may be sufficiently reverse complementary to a target sequence to allow for hybridization, however, may not necessarily be 100% reverse complementary. In some embodiments, a programmable nuclease (e.g., a programmable nickase) may cleave a precursor RNA (“pre-crRNA”) to produce a gRNA, also referred to as a “mature guide RNA.” A programmable nuclease (e.g., a programmable nickase) that cleaves pre-crRNA to produce a mature guide RNA is said to have pre-crRNA processing activity.

A guide nucleic acid (e.g., a crRNA of a gRNA system) can comprise a sequence that is, at least in part, reverse complementary to the sequence of a target nucleic acid. The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthezised. A guide nucleic acid can comprise a crRNA and a tracrRNA or a crRNA and a trancRNA. Sometimes, a guide nucleic acid comprises a crRNA and tracrRNA. The guide nucleic acid can bind specifically to the target nucleic acid. Specifically, the crRNA of the guide nucleic acid may comprise a repeat region and a spacer region. The repeat region hybridizes to a sequence of the tracrRNA and the spacer region hybridizes to a target sequence in a target nucleic acid.

In some embodiments, the tracrRNA sequence comprises at least 70%, at least 80%, at least 90%, at least 92%, at least 95%, at least 97%, or at least 99% sequence identity to any one of SEQ ID NO: 98-SEQ ID NO: 99, SEQ ID NO: 101, SEQ ID NO: 103, SEQ ID NO: 105-SEQ ID NO: 151. In some embodiments, the tracrRNA sequence comprises at least 70% sequence identity to SEQ ID NO: 99. In some embodiments, the tracrRNA sequence comprises at least 75% sequence identity to SEQ ID NO: 99. In some embodiments, the tracrRNA sequence comprises at least 80% sequence identity to SEQ ID NO: 99. In some embodiments, the tracrRNA sequence comprises at least 85% sequence identity to SEQ ID NO: 99. In some embodiments, the tracrRNA sequence comprises at least 90% sequence identity to SEQ ID NO: 99. In some embodiments, the tracrRNA sequence comprises at least 95% sequence identity to SEQ ID NO: 99. In some embodiments, the tracr sequence used in complex with a programmable nickase of the present disclosure comprises SEQ ID NO: 99. In some embodiments, the tracrRNA sequence comprises at least 70% sequence identity to SEQ ID NO: 101. In some embodiments, the tracrRNA sequence comprises at least 75% sequence identity to SEQ ID NO: 101. In some embodiments, the tracrRNA sequence comprises at least 80% sequence identity to SEQ ID NO: 101. In some embodiments, the tracrRNA sequence comprises at least 85% sequence identity to SEQ ID NO: 101. In some embodiments, the tracrRNA sequence comprises at least 90% sequence identity to SEQ ID NO: 101. In some embodiments, the tracr sequence comprises at least 95% sequence identity to SEQ ID NO: 101. In some embodiments, the tracr sequence used in complex with a programmable nickase of the present disclosure comprises SEQ ID NO: 101. In some embodiments, the tracrRNA sequence comprises at least 70% sequence identity to SEQ ID NO: 103. In some embodiments, the tracrRNA sequence comprises at least 75% sequence identity to SEQ ID NO: 103. In some embodiments, the tracrRNA sequence comprises at least 80% sequence identity to SEQ ID NO: 103. In some embodiments, the tracrRNA sequence comprises at least 85% sequence identity to SEQ ID NO: 103. In some embodiments, the tracrRNA sequence comprises at least 90% sequence identity to SEQ ID NO: 103. In some embodiments, the tracrRNA sequence comprises at least 95% sequence identity to SEQ ID NO: 103. In some embodiments, the tracrRNA sequence used in complex with a programmable nickase of the present disclosure comprises SEQ ID NO: 103. In some embodiments, the tracrRNA sequence comprises at least 70% sequence identity to SEQ ID NO: 119. In some embodiments, the tracrRNA sequence comprises at least 75% sequence identity to SEQ ID NO: 119. In some embodiments, the tracrRNA sequence comprises at least 80% sequence identity to SEQ ID NO: 119. In some embodiments, the tracrRNA sequence comprises at least 85% sequence identity to SEQ ID NO: 119. In some embodiments, the tracrRNA sequence comprises at least 90% sequence identity to SEQ ID NO: 119. In some embodiments, the tracrRNA sequence comprises at least 95% sequence identity to SEQ ID NO: 119. In some embodiments, the tracrRNA sequence used in complex with a programmable nickase of the present disclosure comprises SEQ ID NO: 119.

In some embodiments, the programmable nickase comprises a sequence with 70% identity to SEQ ID NO: 10. In some embodiments, the programmable nickase comprises a sequence with 75% identity to SEQ ID NO: 10. In some embodiments, the programmable nickase comprises a sequence with 80% identity to SEQ ID NO: 10. In some embodiments, the programmable nickase comprises a sequence with 85% identity to SEQ ID NO: 10. In some embodiments, the programmable nickase comprises a sequence with 90% identity to SEQ ID NO: 10. In some embodiments, the programmable nickase comprises a sequence with 95% identity to SEQ ID NO: 10. In some embodiments, the programmable nickase comprises a sequence with 70% identity to SEQ ID NO: 11. In some embodiments, the programmable nickase comprises a sequence with 75% identity to SEQ ID NO: 11. In some embodiments, the programmable nickase comprises a sequence with 80% identity to SEQ ID NO: 11. In some embodiments, the programmable nickase comprises a sequence with 85% identity to SEQ ID NO: 11. In some embodiments, the programmable nickase comprises a sequence with 90% identity to SEQ ID NO: 11. In some embodiments, the programmable nickase comprises a sequence with 95% identity to SEQ ID NO: 11. In some embodiments, the programmable nickase comprises a sequence with 70% identity to SEQ ID NO: 33. In some embodiments, the programmable nickase comprises a sequence with 75% identity to SEQ ID NO: 33. In some embodiments, the programmable nickase comprises a sequence with 80% identity to SEQ ID NO: 33. In some embodiments, the programmable nickase comprises a sequence with 85% identity to SEQ ID NO: 33. In some embodiments, the programmable nickase comprises a sequence with 90% identity to SEQ ID NO: 33. In some embodiments, the programmable nickase comprises a sequence with 95% identity to SEQ ID NO: 33.

In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of a guide nucleic acid that comprises a sequence that is reverse complementary to the target nucleic acid is 20 nucleotides in length. A guide nucleic acid can have at least 10, 11, 12, 13, 14, 15, 16, 17, 18, 19, 20, 21, 22, 23, 24, 25, 26, 27, 28, 29, or 30 nucleotides reverse complementary to a target nucleic acid. In some cases, the guide nucleic acid can be 10, 11, 12, 13, 14, 15, 16, 17, 18, 19, 20, 21, 22, 23, 24, 25, 26, 27, 28, 29, or 30 nucleotides in length. For example, a guide nucleic acid may be at least 10 bases. In some embodiments, a guide nucleic acid may be from 10 to 50 bases. In some embodiments, a guide nucleic acid may be at least 25 bases. In some cases, the guide nucleic acid has from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt reverse complementary to a target nucleic acid. In some cases, the guide nucleic acid has from about 10 nt to about 60 nt, from about 20 nt to about 50 nt, or from about 30 nt to about 40 nt reverse complementary to a target nucleic acid. It is understood that the sequence of a guide nucleic acid need not be 100% reverse complementary to that of its target nucleic acid to be specifically hybridizable, hybridizable, or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid. The guide nucleic acid can hybridize with a target nucleic acid.

The guide nucleic acid (e.g., a non-naturally occurring guide nucleic acid) can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a strain of HPV 16 or HPV18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nickase (e.g., a Cas14a, a Cas14b, or a Cas14e disclosed herein), wherein a guide nucleic acid sequence of the pool of guide nucleic acids has a sequence selected from a group of tiled guide nucleic acid that correspond to nucleic acid sequence of a target nucleic acid; and assaying for a signal produce by cleavage of at least some nucleic acids of a reporter of a population of nucleic acids of a reporter. Pooling of guide nucleic acids can ensure broad spectrum identification, or broad coverage, of a target species within a single reaction. This can be particularly helpful in diseases or indications, like sepsis, that may be caused by multiple organisms.

The compositions disclosed herein may comprise gRNA systems. A gRNA system, as described herein, may comprise a crRNA and a tracrRNA or a trancRNA. In a gRNA system, the crRNA and the tracrRNA or trancRNA may be distinct polyribonucleotides.

In some embodiments, a crRNA in a gRNA system comprises a repeat and a spacer. The repeat may hybridize to a region of a tracrRNA or a trancRNA. The spacer may hybridize to a region of a target nucleic acid.

A tracrRNA or a trancRNA in a gRNA system may comprise a region that hybridizes to a crRNA and a region that interacts with a programmable nuclease (e.g., a programmable nickase).

A programmable nickase of the present disclosure (e.g., a Cas14 protein) may be activated to exhibit cleavage activity (e.g., cis-cleavage of a target nucleic acid or trans-cleavage of a collateral nucleic acid) upon binding of a ribonucleoprotein (RNP) (a complex of a programmable nickase and gRNA) to a target nucleic acid, in which the spacer of the crRNA of the gRNA hybridizes to the target nucleic acid.

In some embodiments, a trancRNA may be used in place of a tracrRNA. Compositions and methods of the present disclosure may include a CasZ transactivating noncoding RNA (“trancRNA”; also referred to herein as a “CasZ trancRNA”). In some cases, a trancRNA forms a complex with a CasZ polypeptide of the present disclosure and a CasZ guide RNA. A trancRNA can be identified as a highly transcribed RNA encoded by a nucleotide sequence present in a CasZ locus. The sequence encoding a trancRNA may be located between the cas genes and the array of the CasZ locus (the repeats) (e.g., can be located adjacent to the repeat sequences). Examples below demonstrate detection of a CasZ trancRNA. In some cases, a CasZ trancRNA co-immunoprecipitates (forms a complex with) with a CasZ polypeptide. In some cases, the presence of a CasZ trancRNA is required for function of the system. Data related to trancRNAs (e.g., their expression and their location on naturally occurring arrays) is presented in the examples section below.

In some cases, a CasZ trancRNA has a length of from 60 nucleotides (nt) to 270 nt (e.g., 60-260, 70-270, 70-260, or 75-255 nt). In some cases, a CasZ trancRNA (e.g., a CasZa trancRNA) has a length of from 60-150 nt (e.g., 60-140, 60-130, 65-150, 65-140, 65-130, 70-150, 70-140, or 70-130 nt). In some cases, a CasZ trancRNA (e.g., a CasZa trancRNA) has a length of from 70-130 nt. In some cases, a CasZ trancRNA (e.g., a CasZa trancRNA) has a length of about 80 nt. In some cases, a CasZ trancRNA (e.g., a CasZa trancRNA) has a length of about 90 nt. In some cases, a CasZ trancRNA (e.g., a CasZa trancRNA) has a length of about 120 nt.

In some cases, a CasZ trancRNA (e.g., a CasZb trancRNA) has a length of from 85-240 nt (e.g., 85-230, 85-220, 85-150, 85-130, 95-240, 95-230, 95-220, 95-150, or 95-130 nt). In some cases, a CasZ trancRNA (e.g., a CasZb trancRNA) has a length of from 95-120 nt. In some cases, a CasZ trancRNA (e.g., a CasZb trancRNA) has a length of about 105 nt. In some cases, a CasZ trancRNA (e.g., a CasZb trancRNA) has a length of about 115 nt. In some cases, a CasZ trancRNA (e.g., a CasZb trancRNA) has a length of about 215 nt.

In some cases, a CasZ trancRNA (e.g., a CasZc trancRNA) has a length of from 80-275 nt (e.g., 85-260 nt). In some cases, a CasZ trancRNA (e.g., a CasZc trancRNA) has a length of from 80-110 nt (e.g., 85-105 nt). In some cases, a CasZ trancRNA (e.g., a CasZc trancRNA) has a length of from 235-270 nt (e.g., 240-260 nt). In some cases, a CasZ trancRNA (e.g., a CasZc trancRNA) has a length of about 95 nt. In some cases, a CasZ trancRNA (e.g., a CasZc trancRNA) has a length of about 250 nt.

Compositions and methods of the present disclosure include a Cas14 transactivating noncoding RNA (“trancRNA”; also referred to herein as a “Cas14 trancRNA”). In some cases, a trancRNA forms a complex with a Cas14 polypeptide of the present disclosure and a Cas14 guide RNA. A trancRNA can be identified as a highly transcribed RNA encoded by a nucleotide sequence present in a Cas14 locus. The sequence encoding a trancRNA is usually located between the cas genes and the array of the Cas14 locus (the repeats) (e.g., can be located adjacent to the repeat sequences). Examples below demonstrate detection of a Cas14 trancRNA. In some cases, a Cas14 trancRNA co-immunoprecipitates (forms a complex with) with a CasZ polypeptide. In some cases, the presence of a CasZ trancRNA is required for function of the system.

In some cases, a Cas14 trancRNA has a length of from 60 nucleotides (nt) to 270 nt (e.g., 60-260, 70-270, 70-260, or 75-255 nt). In some cases, a Cas14 trancRNA (e.g., a Cas14a trancRNA) has a length of from 60-150 nt (e.g., 60-140, 60-130, 65-150, 65-140, 65-130, 70-150, 70-140, or 70-130 nt). In some cases, a Cas14 trancRNA (e.g., a Cas14a trancRNA) has a length of from 70-130 nt. In some cases, a Cas14 trancRNA (e.g., a Cas14a trancRNA) has a length of about 80 nt. In some cases, a Cas14 trancRNA (e.g., a Cas14a trancRNA) has a length of about 90 nt. In some cases, a Cas14 trancRNA (e.g., a Cas14a trancRNA) has a length of about 120 nt.

In some cases, a Cas14 trancRNA (e.g., a Cas14b trancRNA) has a length of from 85-240 nt (e.g., 85-230, 85-220, 85-150, 85-130, 95-240, 95-230, 95-220, 95-150, or 95-130 nt). In some cases, a Cas14 trancRNA (e.g., a Cas14b trancRNA) has a length of from 95-120 nt. In some cases, a Cas14 trancRNA (e.g., a Cas14b trancRNA) has a length of about 105 nt. In some cases, a Cas14 trancRNA (e.g., a Cas14b trancRNA) has a length of about 115 nt. In some cases, a Cas14 trancRNA (e.g., a Cas14b trancRNA) has a length of about 215 nt.

In some cases, a Cas14 trancRNA (e.g., a Cas14c trancRNA) has a length of from 80-275 nt (e.g., 85-260 nt). In some cases, a Cas14 trancRNA (e.g., a Cas14c trancRNA) has a length of from 80-110 nt (e.g., 85-105 nt). In some cases, a Cas14 trancRNA (e.g., a Cas14c trancRNA) has a length of from 235-270 nt (e.g., 240-260 nt). In some cases, a Cas14 trancRNA (e.g., a Cas14c trancRNA) has a length of about 95 nt. In some cases, a Cas14 trancRNA (e.g., a Cas14c trancRNA) has a length of about 250 nt.

### Sample

A wide array of samples are compatible with the compositions and methods disclosed herein. The samples, as described herein, may be used in the methods of nicking a target nucleic acid disclosed herein. The samples, as described herein, may be used in the DETECTR assay methods disclosed herein. The samples, as described herein, are compatible with any of the programmable nickases disclosed herein and use of said programmable nickase in a method of detecting a target nucleic acid. The samples, as described herein, are compatible with any of the compositions comprising a programmable nickase and a buffer. Described herein are sample that contain deoxyribonucleic acid (DNA), ribonucleic acid (RNA), or both, which can be modified or detected using a programmable nickase of the present disclosure. As described herein, programmable nickases are activated upon binding to a target nucleic acid of interest in a sample upon hybridization of a guide nucleic acid to the target nucleic acid. Subsequently, the activated programmable nickases exhibit sequence-independent cleavage of a nucleic acid in a reporter. The reporter additionally includes a detectable moiety, which is released upon sequence-independent cleavage of the nucleic acid in the reporter. The detectable moiety emits a detectable signal, which can be measured by various methods (e.g., spectrophotometry, fluorescence measurements, electrochemical measurements).

Various sample types comprising a target nucleic acid of interest are consistent with the present disclosure. These samples can comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample can be obtained to test for presence of a disease, cancer, genetic disorder, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 or any of value from 1 μl to 500 preferably from 10 μL to 200 μL, or more preferably from 50 μL to 100 μL. Sometimes, the sample is contained in more than 500 μl.

In some embodiments, the target nucleic acid is single-stranded DNA. The methods, reagents, enzymes, and kits disclosed herein may enable the direct detection of a DNA encoding a sequence of interest, in particular a single-stranded DNA encoding a sequence of interest, without transcribing the DNA into RNA, for example, by using an RNA polymerase. The compositions and methods disclosed herein may enable the detection of target nucleic acid that is an amplified nucleic acid of a nucleic acid of interest. In some embodiments, the target nucleic acid is a cDNA, genomic DNA, an amplicon of genomic DNA or a DNA amplicon of an RNA. A nucleic acid can encode a sequence from a genomic locus. In some cases, the target nucleic acid that binds to the guide nucleic acid is from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. The nucleic acid can be from 10 to 90, from 20 to 80, from 30 to 70, or from 40 to 60 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can encode a sequence reverse complementary to a guide nucleic acid sequence.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample described herein may comprise at least one target nucleic acid. The target nucleic acid comprises a segment that is reverse complementary to a segment of a guide nucleic acid. Often, the sample comprises the segment of the target nucleic acid and at least one nucleic acid comprising at least 50% sequence identity to a segment of the target nucleic acid. Sometimes, the at least one nucleic acid comprises a segment comprising at least 60%, 70%, 75%, 80%, 85%, 90%, 91%, 92%, 93%, 94%, 95%, 96%, 97%, 98%, or 99% sequence identity to the segment of the target nucleic acid. Often, a sample comprises the segment of the target nucleic acid and at least one nucleic acid a segment comprising less than 100% sequence identity to the target nucleic acid but no less than 50% sequence identity to the segment of the target nucleic acid. Sometimes, a sample comprises the segment of the target nucleic acid and at least one nucleic acid a segment comprising less than 100% sequence identity to the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. For example, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid. Sometimes, the segment of the target nucleic acid comprises a mutation as compared to at least one nucleic acid comprising a segment comprising less than 100% sequence identity to the segment of the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. Often, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid. The mutation can be a mutation of 1, 2, 3, 4, 5, 6, 7, 8, 9, 10, 11, 12, 13, 14, 15, 16, 17, 18, 19, 20 or more nucleotides. Often, the mutation is a single nucleotide mutation. The single nucleotide mutation can be a single nucleotide polymorphism (SNP), which is a single base pair variation in a DNA sequence present in less than 1% of a population. Sometimes, the target nucleic acid comprises a single nucleotide mutation, wherein the single nucleotide mutation comprises the wild type variant of the SNP. The single nucleotide mutation or SNP can be associated with a phenotype of the sample or a phenotype of the organism from which the sample was taken. The SNP, in some cases, is associated with altered phenotype from wild type phenotype. Often, the segment of the target nucleic acid sequence comprises a deletion as compared to at least one nucleic acid comprising a segment comprising less than 100% sequence identity to the segment of the target nucleic acid but no less than 50% sequence identity to the segment of the target nucleic acid. The mutation can be a deletion of 1, 2, 3, 4, 5, 6, 7, 8, 9, 10, 11, 12, 13, 14, 15, 16, 17, 18, 19, 20 or more nucleotides. The mutation can be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation can be a deletion of from 1 to 5, from 5 to 10, from 10 to 15, from 15 to 20, from 20 to 25, from 25 to 30, from 30 to 35, from 35 to 40, from 40 to 45, from 45 to 50, from 50 to 55, from 55 to 60, from 60 to 65, from 65 to 70, from 70 to 75, from 75 to 80, from 80 to 85, from 85 to 90, from 90 to 95, from 95 to 100, from 100 to 200, from 200 to 300, from 300 to 400, from 400 to 500, from 500 to 600, from 600 to 700, from 700 to 800, from 800 to 900, from 900 to 1000, from 1 to 50, from 1 to 100, from 25 to 50, from 25 to 100, from 50 to 100, from 100 to 500, from 100 to 1000, or from 500 to 1000 nucleotides. The segment of the target nucleic acid that the guide nucleic acid of the methods describe herein binds to comprises the mutation, such as the SNP or the deletion. The mutation can be a single nucleotide mutation or a SNP. The SNP can be a synonymous substitution or a nonsynonymous substitution. The nonsynonymous substitution can be a missense substitution or a nonsense point mutation. The synonymous substitution can be a silent substitution. The mutation can be a deletion of one or more nucleotides. Often, the single nucleotide mutation, SNP, or deletion is associated with a disease such as cancer or a genetic disorder. The mutation, such as a single nucleotide mutation, a SNP, or a deletion, can be encoded in the sequence of a target nucleic acid from the germline of an organism or can be encoded in a target nucleic acid from a diseased cell, such as a cancer cell.

The sample used for disease testing may comprise at least one target nucleic acid that can bind to a guide nucleic acid of the reagents described herein. The sample used for disease testing may comprise at least nucleic acid of interest that is amplified to produce a target nucleic acid that can bind to a guide nucleic acid of the reagents described herein. The nucleic acid of interest can comprise DNA, RNA, or a combination thereof.

The target nucleic acid (e.g., a target DNA) may be a portion of a nucleic acid from a virus or a bacterium or other agents responsible for a disease in the sample. The target nucleic acid may be a portion of a nucleic acid from a gene expressed in a cancer or genetic disorder in the sample. In some cases, the sequence is a segment of a target nucleic acid sequence. A segment of a target nucleic acid sequence can be from a genomic locus, a transcribed mRNA, or a reverse transcribed cDNA. A segment of a target nucleic acid sequence can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A segment of a target nucleic acid sequence can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The sequence of the target nucleic acid segment can be reverse complementary to a segment of a guide nucleic acid sequence. The target nucleic acid may comprise a genetic variation (e.g., a single nucleotide polymorphism), with respect to a standard sample, associated with a disease phenotype or disease predisposition. The target nucleic acid may be an amplicon of a portion of an RNA, may be a DNA, or may be a DNA amplicon from any organism in the sample.

In some embodiments, the target nucleic acid sequence comprises a nucleic acid sequence of a virus or a bacterium or other agents responsible for a disease in the sample. In some embodiments, the target nucleic acid comprises DNA that is reverse transcribed from RNA using a reverse transcriptase prior to detection by a programmable nickase (e.g., a Cas14a, a Cas14b, or a Cas14e disclosed herein) using the compositions, systems, and methods disclosed herein. The target nucleic acid, in some cases, is a portion of a nucleic acid from a sexually transmitted infection or a contagious disease, in the sample. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), Chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment. In some cases, the mutation that confers resistance to a treatment is a deletion.

The sample used for cancer testing may comprise at least one target nucleic acid that can bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay can be used to detect “hotspots” in target nucleic acids that can be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci can be probed for a mutation or deletion using the compositions and methods disclosed herein. For example, in the EGFR gene locus, the compositions and methods for detection disclosed herein can be used to detect a single nucleotide polymorphism or a deletion. The SNP or deletion can occur in a non-coding region or a coding region. The SNP or deletion can occur in an Exon, such as Exon19.

The sample used for genetic disorder testing may comprise at least one target nucleic acid that can bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that can bind to a guide nucleic acid of the reagents described herein. The target nucleic acid, in some cases, is a nucleic acid encoding a sequence associated with a phenotypic trait.

The sample used for genotyping testing may comprise at least one target nucleic acid that can bind to a guide nucleic acid of the reagents described herein. The target nucleic acid, in some cases, is a nucleic acid encoding a sequence associated with a genotype of interest.

The sample used for ancestral testing may comprise at least one target nucleic acid that can bind to a guide nucleic acid of the reagents described herein. The target nucleic acid, in some cases, is a nucleic acid encoding a sequence associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status.

In some instances, the target nucleic acid is a single stranded nucleic acid. Alternatively or in combination, the target nucleic acid is a double stranded nucleic acid and is prepared into single stranded nucleic acids before or upon contacting the reagents. The target nucleic acid may be a reverse transcribed RNA, DNA, DNA amplicon, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is single-stranded DNA (ssDNA) or mRNA. In some cases, the target nucleic acid is from a virus, a parasite, or a bacterium described herein. In some cases, the target nucleic acid is transcribed from a gene as described herein and then reverse transcribed into a DNA amplicon.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample as from 1 to 10,000, from 100 to 8000, from 400 to 6000, from 500 to 5000, from 1000 to 4000, or from 2000 to 3000 target nucleic acids. In some cases, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. Often, the target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. The target nucleic acid can also be from 0.1% to 1% of the total nucleic acids in the sample. The target nucleic acid can be DNA or RNA. The target nucleic acid can be any amount less than 100% of the total nucleic acids in the sample. The target nucleic acid can be 100% of the total nucleic acids in the sample.

In some embodiments, the sample comprises a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of from 1 nM to 2 nM, from 2 nM to 3 nM, from 3 nM to 4 nM, from 4 nM to 5 nM, from 5 nM to 6 nM, from 6 nM to 7 nM, from 7 nM to 8 nM, from 8 nM to 9 nM, from 9 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 2 μM, from 2 μM to 3 μM, from 3 μM to 4 μM, from 4 μM to 5 μM, from 5 μM to 6 μM, from 6 μM to 7 μM, from 7 μM to 8 μM, from 8 μM to 9 μM, from 9 μM to 10 μM, from 10 μM to 100 μM, from 100 μM to 1 mM, from 1 nM to 10 nM, from 1 nM to 100 nM, from 1 nM to 1 μM, from 1 nM to 10 μM, from 1 nM to 100 μM, from 1 nM to 1 mM, from 10 nM to 100 nM, from 10 nM to 1 μM, from 10 nM to 10 μM, from 10 nM to 100 μM, from 10 nM to 1 mM, from 100 nM to 1 μM, from 100 nM to 10 μM, from 100 nM to 100 μM, from 100 nM to 1 mM, from 1 μM to 10 μM, from 1 μM to 100 μM, from 1 μM to 1 mM, from 10 μM to 100 μM, from 10 μM to 1 mM, or from 100 μM to 1 mM. In some embodiments, the sample comprisis a target nucleic acid at a concentration of from 20 nM to 200 μM, from 50 nM to 100 μM, from 200 nM to 50 μM, from 500 nM to 20 μM, or from 2 μM to 10 μM. In some embodiments, the target nucleic acid is not present in the sample.

In some embodiments, the sample comprises fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises from 10 copies to 100 copies, from 100 copies to 1000 copies, from 1000 copies to 10,000 copies, from 10,000 copies to 100,000 copies, from 100,000 copies to 1,000,000 copies, from 10 copies to 1000 copies, from 10 copies to 10,000 copies, from 10 copies to 100,000 copies, from 10 copies to 1,000,000 copies, from 100 copies to 10,000 copies, from 100 copies to 100,000 copies, from 100 copies to 1,000,000 copies, from 1,000 copies to 100,000 copies, or from 1,000 copies to 1,000,000 copies of a target nucleic acid sequence. In some embodiments, the sample comprises from 10 copies to 500,000 copies, from 200 copies to 200,000 copies, from 500 copies to 100,000 copies, from 1000 copies to 50,000 copies, from 2000 copies to 20,000 copies, from 3000 copies to 10,000 copies, or from 4000 copies to 8000 copies. In some embodiments, the target nucleic acid is not present in the sample.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

In some embodiments, the target nucleic acid as disclosed herein can activate the programmable nickase to initiate sequence-independent cleavage of a nucleic acid-based reporter (e.g., a reporter comprising a DNA sequence, a reporter comprising an RNA sequence, or a reporter comprising DNA and RNA). For example, a programmable nickase of the present disclosure is activated by a target DNA to cleave reporters having an RNA (also referred to herein as an “RNA reporter”). Alternatively, a programmable nickase of the present disclosure is activated by a target RNA to cleave reporters having an RNA. Alternatively, a programmable nickase of the present disclosure is activated by a target DNA to cleave reporters having a DNA (also referred to herein as a “DNA reporter”). The RNA reporter can comprise a single-stranded RNA labelled with a detection moiety or can be any RNA reporter as disclosed herein. The DNA reporter can comprise a single-stranded DNA labelled with a detection moiety or can be any DNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

Any of the above disclosed samples are consistent with the methods, compositions, reagents, enzymes, and kits disclosed herein and can be used as a companion diagnostic with any of the diseases disclosed herein, or can be used in reagent kits, point-of-care diagnostics, or over-the-counter diagnostics.

### Methods of Nicking of a Target Nucleic Acid

Disclosed herein are methods of introducing a break into a target nucleic acid. In some embodiments, the break may be a single stranded break (e.g., a nick). The programmable nickases (e.g., Cas14a, Cas14b, or Cas14e disclosed herein) and gRNA systems (e.g., a gRNA comprising a crRNA and a tracrRNA or a gRNA comprising a crRNA and a tracnRNA) disclosed herein may be used to introduce a break into a target nucleic acid. A method of introducing a break into a target nucleic acid may comprise contacting the target nucleic acid with a first guide nucleic acid (e.g., a guide nucleic acid comprising a first region that binds to a first programmable nickase having a length of no more than 900 amino acids) and a second guide nucleic acid (e.g., a guide nucleic acid comprising a first region that binds to a second programmable nickase having a length of no more than 900 amino acids). The first guide nucleic acid may comprise a second region that binds to the target nucleic acid, and the second guide nucleic acid may comprise a second region that binds to the target nucleic acid. The second region of the first guide nucleic acid and the second region of the second guide nucleic acid may bind opposing strands of the target nucleic acid.

The methods described herein (e.g., methods of introducing a nick into a target nucleic acid) may be used to modify a target nucleic acid. Methods of modifying a target nucleic acid may use the compositions comprising a programmable nickase (e.g., a Cas14 protein) and a gRNA system (e.g., a gRNA system comprising a crRNA and a tracrRNA or gRNA system comprising a crRNA and a trancRNA) described herein. Modifying a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid. The target nucleic acid may comprise one or more of a genome, a chromosome, a plasmid, a gene, a promoter, an untranslated region, an open reading frame, an intron, an exon, or an operator. The target nucleic acid may comprise a segment of one or more of a genome, a chromosome, a plasmid, a gene, a promoter, an untranslated region, an open reading frame, an intron, an exon, or an operator. In some embodiments, the target nucleic acid may be part of a cell or an organism. In some embodiments, the target nucleic acid may be a cell-free genetic component. In some embodiments, modifying a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may inserted into the target nucleic acid.

Modifying a target nucleic acid may comprise introducing a break (e.g., a single stranded break) in the target nucleic acid. In some embodiments, a break may be introduced by contacting a target nucleic acid with a programmable nickase (e.g., a Cas14 programmable nickase) a guide nucleic acid. The guide nucleic acid may bind to the programmable nickase and hybridize to a region of the target nucleic acid, thereby recruiting the programmable nickase to the region of the target nucleic acid. Binding of the programmable nickase to the guide nucleic acid and the region of the target nucleic acid may activate the programmable nickase, and the programmable nickase may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first programmable nickase and hybridizes to a first region of the target nucleic acid and a second guide nucleic acid that binds to a second programmable nickase and hybridizes to a second region of the target nucleic acid. The first programmable nickase may introduce a first break in a first strand at the first region of the target nucleic acid, and the second programmable nickase may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with an insert sequence), thereby modifying the target nucleic acid.

A programmable nickase for use in modifying a target nucleic acid may have greater nicking activity as compared to double stranded cleavage activity. In some embodiments, a programmable nickase may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater nicking activity as compared to double stranded cleavage activity.

In other cases, a programmable nickase for use in modifying a target nucleic acid may have greater double stranded cleavage activity as compared to nicking activity. In some embodiments, a programmable nickase may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater double stranded cleavage activity as compared to nicking activity.

In some embodiments, the nicking activity and double stranded cleavage activity of a programmable nickase depend on the conditions and species present in the sample containing the programmable nickase. In some cases, the nicking activity and double stranded cleavage activity of the programmable nickase are responsive to the sequences of the tracrRNAs present. In some cases, the ratio of nicking activity and double stranded cleavage activity can be modulated by changing the sequences of the tracrRNAs present. In some cases, the nicking activity and double stranded cleavage activity of the programmable nickase respond differently to changes in temperature, pH, osmolarity, buffer, target nucleic acid concentration, ionic strength, and inhibitor concentration. In some embodiments, the ratio of nicking activity to cleavage activity by a programmable nickase can be actively controlled by adjusting sample conditions and tracrRNA sequences.

The compositions and methods described herein may be used to treat, prevent, or inhibit an ailment in a subject. For example, a method comprising introducing a nick into a target nucleic acid by contacting the target nucleic acid to a composition comprising a programmable nickase may be used to treat, prevent, or inhibit an ailment in a subject. The ailments may include diseases, cancers, genetic disorders, neoplasias, and infections. In some cases, the ailments are associated with one or more genetic sequences, including but not limited to 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis; anauxetic dysplasia; androgen insentivity syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT1 deficiency; glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborm errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome Imag; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1 Imag; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann corneal dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

In some embodiments, treating, preventing, or inhibiting an ailment in a subject may comprise contacting a target nucleic acid associated with a particular ailment to a programmable nickase (e.g., a Cas14a, a Cas14b, or a Cas14e programmable nickase). In some aspects, the methods of treating, preventing, or inhibiting an ailment may involve removing, modifying, replacing, transposing, or affecting the regulation of a genomic sequence of a patient in need thereof. In some embodiments, the methods of treating, preventing, or inhibiting an ailment may involve modulating gene expression. In some embodiments, the methods of treating, preventing, or inhibiting an ailment may comprise targeting a nucleic acid sequence associated with a pathogen, such as a virus or bacteria, to a programmable nickase of the present disclosure.

The compositions and methods described herein may be used to treat, prevent, diagnose, or identify a cancer in a subject. For example, a method comprising introducing a nick into a target nucleic acid by contacting the target nucleic acid to a composition comprising a programmable nickase may be used to treat, prevent, diagnose, or identify a cancer in a subject. In some aspects, the methods may target cells or tissues. In some embodiments, the methods may be applied to subjects, such as humans. As used herein, the term “cancer” refers to a physiological condition that may be characterized by abnormal or unregulated cell growth or activity. In some cases, cancer may involve the spread of the cells exhibiting abnormal or unregulated growth or activity between various tissues in a subject. In some aspects, cancer may be a genetic condition. Examples of cancers include, but are not limited to Acute Lymphoblastic Leukemia, Acute Myeloid Leukemia, Adrenocortical Carcinoma, Anal Cancer, Astrocytomas, Bile Duct Cancer, Bladder Cancer, Bone Cancer, Brain Cancer, Breast Cancer, Bronchial Cancer, Burkitt Lymphoma, Carcinoma, Cardiac Tumors, Cervical Cancer, Chordoma, Chronic Lymphocytic Leukemia, Chronic Myelogenous Leukemia, Chronic Myeloproliferative Neoplasms, Colon Cancer, Colorectal Cancer, Craniopharyngioma, Cutaneous T-cell lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumors, Extragonadal Germ Cell Tumors, Fallopian Tube Cancer, Fibrous Histiocytoma, Gallbladder Cancer, Gastric Cancer, Gastrointestinal Cancer, Gastrointestinal Carcinoid Cancer, Gastrointestinal Stromal Tumors, Gestational Trophoblastic Disease, Hairy Cell Leukemia, Head and Neck Cancer, Heart Tumors, Hepatocellular Cancer, Histiocytosis, Hodgkin Lymphoma, Hypopharyngeal Cancer, Intraocular Melanoma, Islet Cell Tumors, Kaposi Sarcoma, Kidney cancer, Langerhans Cell Histiocytosis, Laryngeal Cancer, Leukemia, Lip and Oral Cavity Cancer, Liver Cancer, Lung Cancer, Lymphoma, Malignant Fibrous Histiocytoma, Melanoma, Merkel Cell Carcinoma, Mesothelioma, Metastatic Squamous Neck Cancer, Midline Tract Carcinoma, Mouth Cancer, Multiple Endocrine Neoplasia Syndromes, Multiple Myeloma, Mycosis Fungoides, Myelodysplastic Syndromes, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Osteosarcoma, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Renal Cell Cancer, Retinoblastoma, Rhabdomyosarcoma, Salivary Gland Cancer, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Stomach Cancer, T-Cell Lymphoma, Testicular Cancer, Throat Cancer, Thymoma and Thymic Carcinoma, Thyroid Cancer, Tracheobronchial Cancer, Transitional Cell Cancer of the Renal Pelvis and Ureter, Ureter Cancer, Renal Pelvis Cancer, Urethral Cancer, Uterine Cancer, Uterine Sarcoma, Vaginal Cancer, Vascular Tumors, Vulvar Cancer, and Wilms Tumor.

In some cases, a cancer is associated with a particular biomarkers. A biomarker is a chemical species or profile that indicates that may serve as an indicator of a cellular or organismal state (e.g., the presence or absence of a disease). Nonlimiting examples of biomarkers include biomolecules, nucleic acid sequences, proteins, metabolites, nucleic acids, protein modifications. A biomarker may refer to one species or to a plurality of species, such as a cell surface profile.

The methods of the present disclosure (e.g., methods of modifying a target nucleic acid) may comprise targeting a biomarker or a nucleic acid associated with a biomarker with a programmable nickase (e.g., a Cas14a, a Cas14b, or a Cas14e programmable nickase). In some cases, the biomarker is a gene associated with a cancer. Non-limiting examples of genes associated with cancers include, ABL, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CDC73, CDH1, CDK4, CDKN1B, CDKN1C, CDKN2A, CEBPA, CHEK2, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FLCN, FMS, FOS, FPS, GATA2, GLI, GPGSP, GREM1, HER2/neu, HOX11, HOXB13, HST, IL-3, INT-2, JUN, KIT, KS3, K-SAM, LBC, LCK, LMO1, LMO2, L-MYC, LYL-1, LYT-10, LYT-10/Cal, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PDGFRA, PHOX2B, PIM-1, PMS2, POLD1, POLE, POT1, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TMEM127, TP53, TSC1, TSC2, TRK, VHL, WRN, and WT1. In some cases, a gene biomarker for cancer will carry one or more mutations. In some cases, a gene biomarker for a cancer will be upregulated or downregulated relative to a patient or sample that does not have the cancer.

### Detection of a Target Nucleic Acid

The present disclosure provides methods and compositions, which enable target DNA detection by programmable nickase platforms, such as the DNA Endonuclease Targeted CRISPR TransReporter (DETECTR) platform. In some embodiments, target DNA from a sample is amplified before assaying for cleavage of reporters. Target DNA can be amplified by PCR or isothermal amplification techniques. DNA amplification methods that are compatible with the DETECTR technology can be used for programmable nucleases, such as programmable nickases. For example, ssDNA can be amplified. Amplification of ssDNA instead of dsDNA enables PAM-independent detection of nucleic acids by proteins with PAM requirements for dsDNA-activated trans-cleavage, as is the case for some Cas14 proteins.

Certain programmable nucleases (e.g., Cas14 programmable nickases) exhibit indiscriminate trans-cleavage of ssDNA, enabling their use for detection of DNA in samples. In some embodiments, target ssDNA are generated from many nucleic acid templates (RNA, ss/dsDNA) in order to achieve cleavage of the FQ reporter in the DETECTR platform. Certain programmable nickases (e.g., Cas14a1) are activated by ssDNA, upon which they can exhibit trans-cleavage of ssDNA and can, thereby, be used to cleave ssDNA FQ reporter molecules in the DETECTR system. These programmable nickases target ssDNA present in the sample, or generated and/or amplified from any number of nucleic acid templates (RNA, ssDNA, or dsDNA).

In some embodiments, the programmable nickases disclosed herein are used in conjunction with a tracrRNA. In some embodiments, the tracrRNA sequence comprises at least 70%, at least 80%, at least 90%, at least 92%, at least 95%, at least 97%, or at least 99% sequence identity to any one of SEQ ID NO: 98-SEQ ID NO: 99, SEQ ID NO: 101, SEQ ID NO: 103, SEQ ID NO: 105-SEQ ID NO: 151. In some embodiments, the tracrRNA sequence comprises at least 70% sequence identity to SEQ ID NO: 99. In some embodiments, the tracrRNA sequence comprises at least 75% sequence identity to SEQ ID NO: 99. In some embodiments, the tracrRNA sequence comprises at least 80% sequence identity to SEQ ID NO: 99. In some embodiments, the tracrRNA sequence comprises at least 85% sequence identity to SEQ ID NO: 99. In some embodiments, the tracrRNA sequence comprises at least 90% sequence identity to SEQ ID NO: 99. In some embodiments, the tracrRNA sequence comprises at least 95% sequence identity to SEQ ID NO: 99. In some embodiments, the tracrRNA sequence used in complex with a programmable nickase of the present disclosure comprises SEQ ID NO: 99. In some embodiments, the tracrRNA sequence comprises at least 70% sequence identity to SEQ ID NO: 101. In some embodiments, the tracrRNA sequence comprises at least 75% sequence identity to SEQ ID NO: 101. In some embodiments, the tracrRNA sequence comprises at least 80% sequence identity to SEQ ID NO: 101. In some embodiments, the tracrRNA sequence comprises at least 85% sequence identity to SEQ ID NO: 101. In some embodiments, the tracrRNA sequence comprises at least 90% sequence identity to SEQ ID NO: 101. In some embodiments, the tracrRNA sequence comprises at least 95% sequence identity to SEQ ID NO: 101. In some embodiments, the tracrRNA sequence used in complex with a programmable nickase of the present disclosure comprises SEQ ID NO: 101. In some embodiments, the tracrRNA sequence comprises at least 70% sequence identity to SEQ ID NO: 103. In some embodiments, the tracrRNA sequence comprises at least 75% sequence identity to SEQ ID NO: 103. In some embodiments, the tracrRNA sequence comprises at least 80% sequence identity to SEQ ID NO: 103. In some embodiments, the tracrRNA sequence comprises at least 85% sequence identity to SEQ ID NO: 103. In some embodiments, the tracrRNA sequence comprises at least 90% sequence identity to SEQ ID NO: 103. In some embodiments, the tracrRNA sequence comprises at least 95% sequence identity to SEQ ID NO: 103. In some embodiments, the tracrRNA sequence used in complex with a programmable nickase of the present disclosure comprises SEQ ID NO: 103. In some embodiments, the tracrRNA sequence comprises at least 70% sequence identity to SEQ ID NO: 119. In some embodiments, the tracrRNA sequence comprises at least 75% sequence identity to SEQ ID NO: 119. In some embodiments, the tracrRNA sequence comprises at least 80% sequence identity to SEQ ID NO: 119. In some embodiments, the tracrRNA sequence comprises at least 85% sequence identity to SEQ ID NO: 119. In some embodiments, the tracrRNA sequence comprises at least 90% sequence identity to SEQ ID NO: 119. In some embodiments, the tracrRNA sequence comprises at least 95% sequence identity to SEQ ID NO: 119. In some embodiments, the tracrRNA sequence used in complex with a programmable nickase of the present disclosure comprises SEQ ID NO: 119.

In some embodiments, the programmable nickase comprises a sequence with 70% identity to SEQ ID NO: 10. In some embodiments, the programmable nickase comprises a sequence with 75% identity to SEQ ID NO: 10. In some embodiments, the programmable nickase comprises a sequence with 80% identity to SEQ ID NO: 10. In some embodiments, the programmable nickase comprises a sequence with 85% identity to SEQ ID NO: 10. In some embodiments, the programmable nickase comprises a sequence with 90% identity to SEQ ID NO: 10. In some embodiments, the programmable nickase comprises a sequence with 95% identity to SEQ ID NO: 10. In some embodiments, the programmable nickase comprises a sequence with 70% identity to SEQ ID NO: 11. In some embodiments, the programmable nickase comprises a sequence with 75% identity to SEQ ID NO: 11. In some embodiments, the programmable nickase comprises a sequence with 80% identity to SEQ ID NO: 11. In some embodiments, the programmable nickase comprises a sequence with 85% identity to SEQ ID NO: 11. In some embodiments, the programmable nickase comprises a sequence with 90% identity to SEQ ID NO: 11. In some embodiments, the programmable nickase comprises a sequence with 95% identity to SEQ ID NO: 11. In some embodiments, the programmable nickase comprises a sequence with 70% identity to SEQ ID NO: 33. In some embodiments, the programmable nickase comprises a sequence with 75% identity to SEQ ID NO: 33. In some embodiments, the programmable nickase comprises a sequence with 80% identity to SEQ ID NO: 33. In some embodiments, the programmable nickase comprises a sequence with 85% identity to SEQ ID NO: 33. In some embodiments, the programmable nickase comprises a sequence with 90% identity to SEQ ID NO: 33. In some embodiments, the programmable nickase comprises a sequence with 95% identity to SEQ ID NO: 33.

The compositions, kits and methods disclosed herein may be implemented in methods of assaying for a target nucleic acid. In some embodiments, a method of assaying for a target nucleic acid in a sample, comprises: contacting the sample to a complex comprising a guide nucleic acid comprising a segment that is reverse complementary to a segment of the target nucleic acid and a programmable nickase (e.g., a Cas14a, a Cas14b, or a Cas14e disclosed herein) that exhibits sequence independent cleavage upon forming a complex comprising the segment of the guide nucleic acid binding to the segment of the target nucleic acid, wherein the sample comprises at least one nucleic acid comprising at least 50% sequence identity to the segment of the target nucleic acid; and assaying for cleavage of at least one reporter nucleic acids of a population of reporter nucleic acids, wherein the cleavage indicates a presence of the target nucleic acid in the sample and wherein absence of the cleavage indicates an absence of the target nucleic acid in the sample. The target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

The concentrations of the various reagents in the programmable nickase DETECTR reaction mix can vary depending on the particular scale of the reaction. For example, the final concentration of the programmable nickase can vary from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The final concentration of the sgRNA complementary to the target nucleic acid can be from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The concentration of the ssDNA-FQ reporter can be from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM.

An example of a Cas14 DETECTR reaction consists of a final concentration of 100 nM Cas14, 125 nM sgRNA, and 50 nM ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm). The fluorescence wavelength detected can vary depending on the reporter molecule.

Described herein are reagents comprising a single stranded reporter nucleic acid comprising a detection moiety, wherein the reporter nucleic acid (e.g., the ssDNA-FQ reporter described above) is capable of being cleaved by the programmable nickase, upon generation and amplification of ssDNA from a nucleic acid template using the methods disclosed herein, thereby generating a first detectable signal.

The methods disclosed herein, thus, include generation and amplification of ssDNA from a target nucleic acid template (e.g., cDNA, ssDNA, or dsDNA) of interest in a sample, incubation of the ssDNA with an ssDNA activated programmable nickase leading to indiscriminate, PAM-independent cleavage of reporter nucleic acids (also referred to as ssDNA-FQ reporters) to generate a detectable signal, and quantification of the detectable signal to detect a target nucleic acid sequence of interest.

### Reporters

Described herein are reagents comprising a reporter. The reporter can comprise a single stranded nucleic acid and a detection moiety (e.g., a labeled single stranded DNA reporter), wherein the nucleic acid is capable of being cleaved by the activated programmable nickase, releasing the detection moiety, and, generating a detectable signal. As used herein, “reporter” is used interchangeably with “reporter nucleic acid” or “reporter molecule”. The programmable nickases disclosed herein, activated upon hybridization of a guide RNA to a target nucleic acid, can cleave the reporter. Cleaving the “reporter” may be referred to herein as cleaving the “reporter nucleic acid,” the “reporter molecule,” or the “nucleic acid of the reporter.”

A major advantage of the compositions and methods disclosed herein is the design of excess reporters to total nucleic acids in an unamplified or an amplified sample, not including the nucleic acid of the reporter. Total nucleic acids can include the target nucleic acids and non-target nucleic acids, not including the nucleic acid of the reporter. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nickase may be inhibited in its ability to bind and cleave the reporter sequences. This is because the activated programmable nickases collaterally cleaves any nucleic acids. If total nucleic acids are in present in large amounts, they may outcompete reporters for the programmable nickases. The compositions and methods disclosed herein are designed to have an excess of reporter to total nucleic acids, such that the detectable signals from DETECTR reactions are particularly superior. In some embodiments, the reporter can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

A second significant advantage of the compositions and methods disclosed herein is the design of an excess volume comprising the guide nucleic acid, the programmable nickase, and the reporter, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nickase to become activated or to find and cleave the nucleic acid of the reporter. This may be due to nucleic acids that are not the reporter outcompeting the nucleic acid of the reporter, for the programmable nickase. Alternatively, various reagents in the sample may simply inhibit the activity of the programmable nickase. Thus, the compositions and methods provided herein for contacting an excess volume comprising the guide nucleic acid, the programmable nickase, and the reporter to a smaller volume comprising the sample with the target nucleic acid of interest provides for superior detection of the target nucleic acid by ensuring that the programmable nickase is able to find and cleaves the nucleic acid of the reporter. In some embodiments, the volume comprising the guide nucleic acid, the programmable nickase, and the reporter (can be referred to as “a second volume”) is 4-fold greater than a volume comprising the sample (can be referred to as “a first volume”). In some embodiments, the volume comprising the guide nucleic acid, the programmable nickase, and the reporter (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 μL μL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 μL. In some embodiments, the volume comprising the programmable nickase, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 μL μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

In some cases, the reporter nucleic acid is a single-stranded nucleic acid sequence comprising deoxyribonucleotides. In other cases, the reporter nucleic acid is a single-stranded nucleic acid sequence comprising ribonucleotides. The nucleic acid of a reporter can be a single-stranded nucleic acid sequence comprising at least one deoxyribonucleotide and at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, 3, 4, 5, 6, 7, 8, 9, or 10 ribonucleotide residues at an internal position. In some cases, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter has only deoxyribonucleotide residues. In some cases, the nucleic acid comprises nucleotides resistant to cleavage by the programmable nickase described herein. In some cases, the nucleic acid of a reporter comprises synthetic nucleotides. In some cases, the nucleic acid of a reporter comprises at least one ribonucleotide residue and at least one non-ribonucleotide residue. In some cases, the nucleic acid of a reporter is 5-20, 5-15, 5-10, 7-20, 7-15, or 7-10 nucleotides in length. In some cases, the nucleic acid of a reporter is from 3 to 20, from 4 to 10, from 5 to 10, or from 5 to 8 nucleotides in length. In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotide. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotide. In some cases, the nucleic acid of a reporter comprises at least one guanine ribonucleotide. In some cases, the nucleic acid of a reporter comprises at least two guanine ribonucleotide. A nucleic acid of a reporter can comprise only unmodified ribonucleotides, only unmodified deoxyribonucleotides, or a combination thereof. In some cases, the nucleic acid of a reporter is from 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

The single stranded nucleic acid of a reporter comprises a detection moiety capable of generating a first detectable signal. Sometimes the reporter nucleic acid comprises a protein capable of generating a signal. A signal can be a calorimetric, potentiometric, amperometric, optical (e.g., fluorescent, colorimetric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of a reporter. In some cases, the single-stranded nucleic acid of a reporter is at least one population of the single-stranded nucleic acid capable of generating a first detectable signal. In some cases, the single-stranded nucleic acid of a reporter is a population of the single stranded nucleic acid capable of generating a first detectable signal. Optionally, there is more than one population of single-stranded nucleic acid of a reporter. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded nucleic acids of a reporter capable of generating a detectable signal. In some cases, there are from 2 to 50, from 3 to 40, from 4 to 30, from 5 to 20, or from 6 to 10 different populations of single-stranded nucleic acids of a reporter capable of generating a detectable signal.

TABLE 2
Examples of Single Stranded Nucleic Acids in a Reporter

| 5′ Detection Moiety* | Sequence (SEQ ID NO) | 3′ Quencher* |
|---|---|---|
| /56-FAM/ | rUrUrUrUrU (SEQ ID NO: 153) | /3IABkFQ/ |
| /5IRD700/ | rUrUrUrUrU (SEQ ID NO: 153) | /3IRQC1N/ |
| /5TYE665/ | rUrUrUrUrU (SEQ ID NO: 153) | /3IAbRQSp/ |
| /5Alex594N/ | rUrUrUrUrU (SEQ ID NO: 153) | /3IAbRQSp/ |
| /5ATTO633N/ | rUrUrUrUrU (SEQ ID NO: 153) | /3IAbRQSp/ |
| /56-FAM/ | rUrUrUrUrUrUrUrU(SEQ ID NO: 154) | /3IABkFQ/ |
| /5IRD700/ | rUrUrUrUrUrUrUrU(SEQ ID NO: 154) | /3IRQC1N/ |
| /5TYE665/ | rUrUrUrUrUrUrUrU(SEQ ID NO: 154) | /3IAbRQSp/ |
| /5Alex594N/ | rUrUrUrUrUrUrUrU(SEQ ID NO: 154) | /3IAbRQSp/ |
| /5ATTO633N/ | rUrUrUrUrUrUrUrU(SEQ ID NO: 154) | /3IAbRQSp/ |
| /56-FAM/ | rUrUrUrUrUrUrUrUrUrU(SEQ ID NO: 155) | /3IABkFQ/ |
| /5IRD700/ | rUrUrUrUrUrUrUrUrUrU(SEQ ID NO: 155) | /3IRQC1N/ |
| /5TYE665/ | rUrUrUrUrUrUrUrUrUrU(SEQ ID NO: 155) | /3IAbRQSp/ |
| /5Alex594N/ | rUrUrUrUrUrUrUrUrUrU(SEQ ID NO: 155) | /3IAbRQSp/ |
| /5ATTO633N/ | rUrUrUrUrUrUrUrUrUrU(SEQ ID NO: 155) | /3IAbRQSp/ |
| /56-FAM/ | TTTTrUrUTTTT(SEQ ID NO: 156) | /3IABkFQ/ |
| /5IRD700/ | TTTTrUrUTTTT(SEQ ID NO: 156) | /3IRQC1N/ |
| /5TYE665/ | TTTTrUrUTTTT(SEQ ID NO: 156) | /3IAbRQSp/ |
| /5Alex594N/ | TTTTrUrUTTTT(SEQ ID NO: 156) | /3IAbRQSp/ |
| /5ATTO633N/ | TTTTrUrUTTTT(SEQ ID NO: 156) | /3IAbRQSp/ |
| /56-FAM/ | TTrUrUTT(SEQ ID NO: 157) | /3IABkFQ/ |
| /5IRD700/ | TTrUrUTT(SEQ ID NO: 157) | /3IRQC1N/ |
| /5TYE665/ | TTrUrUTT(SEQ ID NO: 157) | /3IAbRQSp/ |
| /5Alex594N/ | TTrUrUTT(SEQ ID NO: 157) | /3IAbRQSp/ |
| /5ATTO633N/ | TTrUrUTT(SEQ ID NO: 157) | /3IAbRQSp/ |
| /56-FAM/ | TArArUGC(SEQ ID NO: 158) | /3IABkFQ/ |
| /5IRD700/ | TArArUGC(SEQ ID NO: 158) | /3IRQC1N/ |
| /5TYE665/ | TArArUGC(SEQ ID NO: 158) | /3IAbRQSp/ |
| /5Alex594N/ | TArArUGC(SEQ ID NO: 158) | /3IAbRQSp/ |
| /5ATTO633N/ | TArArUGC(SEQ ID NO: 158) | /3IAbRQSp/ |
| /56-FAM/ | TArUrGGC(SEQ ID NO: 159) | /3IABkFQ/ |
| /5IRD700/ | TArUrGGC(SEQ ID NO: 159) | /3IRQC1N/ |
| /5TYE665/ | TArUrGGC(SEQ ID NO: 159) | /3IAbRQSp/ |
| /5Alex594N/ | TArUrGGC(SEQ ID NO: 159) | /3IAbRQSp/ |
| /5ATTO633N/ | TArUrGGC(SEQ ID NO: 159) | /3IAbRQSp/ |
| /56-FAM/ | rUrUrUrUrU(SEQ ID NO: 160) | /3IABkFQ/ |
| /5IRD700/ | rUrUrUrUrU(SEQ ID NO: 160) | /3IRQC1N/ |
| /5TYE665/ | rUrUrUrUrU(SEQ ID NO: 160) | /3IAbRQSp/ |
| /5Alex594N/ | rUrUrUrUrU(SEQ ID NO: 160) | /3IAbRQSp/ |
| /5ATTO633N/ | rUrUrUrUrU(SEQ ID NO: 160) | /3IAbRQSp/ |
| /56-FAM/ | TTATTATT (SEQ ID NO: 161) | /3IABkFQ/ |
| /56-FAM/ | TTATTATT (SEQ ID NO: 161) | /3IABkFQ/ |
| /5IRD700/ | TTATTATT (SEQ ID NO: 161) | /3IRQC1N/ |
| /5TYE665/ | TTATTATT (SEQ ID NO: 161) | /3IAbRQSp/ |
| /5Alex594N/ | TTATTATT (SEQ ID NO: 161) | /3IAbRQSp/ |
| /5ATTO633N/ | TTATTATT (SEQ ID NO: 161) | /3IAbRQSp/ |
| /56-FAM/ | TTTTTT (SEQ ID NO: 162) | /3IABkFQ/ |
| /56-FAM/ | TTTTTTTT (SEQ ID NO: 163) | /3IABkFQ/ |
| /56-FAM/ | TTTTTTTTTT (SEQ ID NO: 164) | /3IABkFQ/ |
| /56-FAM/ | TTTTTTTTTTTT (SEQ ID NO: 165) | /3IABkFQ/ |
| /56-FAM/ | TTTTTTTTTTTTTT(SEQ ID NO: 166) | /3IABkFQ/ |
| /56-FAM/ | AAAAAA (SEQ ID NO: 167) | /3IABkFQ/ |
| /56-FAM/ | CCCCCC (SEQ ID NO: 168) | /3IABkFQ/ |
| /56-FAM/ | GGGGGG (SEQ ID NO: 169) | /3IABkFQ/ |
| /56-FAM/ | TTATTATT (SEQ ID NO: 161) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE665 (Integrated DNA Technologies)
/5Alex594N/: 5′ Alexa Fluor 594 (NHSEster)(Integrated DNA Technologies)
/5ATTO633N/: 5′ ATTO TM633 (NHSEster)(Integrated DNA Technologies)
/3IRQC1N/: 3′ IRDye QC-1 Quencher (Li-Cor)
/3IAbRQSp/: 3′ Iowa Black RQ(Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a detectable fluorescence signal in the same range as 6-Fluorescein, IRDye 700, TYE 665, Alex Fluor, or ATTO™ 633 (NHS Ester). A detection moiety can be fluorescein amidite, 6-Fluorescein, IRDye 700, TYE 665, Alex Fluor 594, or ATTO™ 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 153 with a fluorophore that emits a fluorescence around 520 nm is used for testing in non-urine samples, and SEQ ID NO: 160 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO™ 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nickases has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal can be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal can be a colorimetric signal or a signal visible by eye. In some instances, the detectable signal can be fluorescent, electrical, chemical, electrochemical, or magnetic. In some cases, the first detection signal can be generated by binding of the detection moiety to the capture molecule in the detection region, where the first detection signal indicates that the sample contained the target nucleic acid. Sometimes the system can be capable of detecting more than one type of target nucleic acid, wherein the system comprises more than one type of guide nucleic acid and more than one type of reporter nucleic acid. In some cases, the detectable signal can be generated directly by the cleavage event. Alternatively or in combination, the detectable signal can be generated indirectly by the signal event. Sometimes the detectable signal is not a fluorescent signal. In some instances, the detectable signal can be a colorimetric or color-based signal. In some cases, the detected target nucleic acid can be identified based on its spatial location on the detection region of the support medium. In some cases, the second detectable signal can be generated in a spatially distinct location than the first generated signal.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose. A DNS reagent produces a colorimetric change when invertase converts sucrose to glucose. In some cases, it is preferred that the nucleic acid (e.g., DNA) and invertase are conjugated using a heterobifunctional linker via sulfo-SMCC chemistry. Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of nucleic acid of a reporter. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, from 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, the methods, compositions, reagents, enzymes, and kits described herein may be used to detect a target single-stranded nucleic acid in a sample where the sample is contacted with the reagents for a predetermined length of time sufficient for the trans-cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 2 hours, or from 45 minutes to 1 hour.

When a guide nucleic acid binds to a target nucleic acid, the programmable nickase's trans-cleavage activity can be initiated, and nucleic acids of a reporter can be cleaved, resulting in the detection of fluorescence. The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthezised. Nucleic acid reporters can comprise a detection moiety, wherein the nucleic acid reporter can be cleaved by the activated programmable nickase, thereby generating a signal. Some methods as described herein can a method of assaying for a target nucleic acid in a sample comprises contacting the sample to a complex comprising a guide nucleic acid comprising a segment that is reverse complementary to a segment of the target nucleic acid and a programmable nickase that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the nucleic acid of a reporter using the programmable nickase may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nickase capable of being activated when complexed with the guide nucleic acid and the target nucleic acid segment, a single stranded nucleic acid of a reporter comprising a detection moiety, wherein the nucleic acid of a reporter is capable of being cleaved by the activated programmable nickase, thereby generating a first detectable signal, cleaving the single stranded nucleic acid of a reporter using the programmable nickase that cleaves as measured by a change in color, and measuring the first detectable signal on the support medium. The cleaving of the single stranded nucleic acid of a reporter using the programmable nickase may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nickase capable of being activated when complexed with the guide nucleic acid and the target nucleic acid segment, and a single stranded nucleic acid of a reporter comprising a detection moiety, wherein the nucleic acid of a reporter is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample.

In some cases, the methods, reagents, enzymes, and kits described herein detect a target single-stranded nucleic acid with a programmable nickase and a single-stranded nucleic acid of a reporter in a sample where the sample is contacted with the reagents for a predetermined length of time sufficient for trans-cleavage of the single stranded nucleic acid of a reporter.

Some methods as described herein can be a method of detecting a target nucleic acid in a sample comprising contacting the sample comprising the target nucleic acid with a guide nucleic acid targeting a target sequence, a programmable nickase capable of being activated when complexed with the guide nucleic acid and the target sequence, a single stranded reporter nucleic acid comprising a detection moiety, wherein the reporter nucleic acid is capable of being cleaved by the activated nuclease, thereby generating a first detectable signal, cleaving the single stranded reporter nucleic acid using the programmable nickase that cleaves as measured by a change in color, and measuring the first detectable signal on the support medium. The cleaving of the single stranded reporter nucleic acid using the programmable nickase may cleave with an efficiency of 50% as measured by a change in color. In some cases, the cleavage efficiency is at least 40%, at least 50%, at least 60%, at least 70%, at least 80%, at least 90%, or at least 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target sequence, a programmable nickase capable of being activated when complexed with the guide nucleic acid and the target sequence, and a single stranded reporter nucleic acid comprising a detection moiety, wherein the reporter nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

### Multiplexing Programmable Nucleases and Programmable Nickases

Described herein are reagents comprising a programmable nuclease capable of being activated when complexed with the guide nucleic acid and the target nucleic acid molecule. Furthermore, these reagents can be used with different types of programmable nuclease, e.g., for multiplexing programmable nucleases. In some embodiments, a programmable nickase (e.g., a Cas14 programmable nickase) may be multiplexed with an additional programmable nuclease. For example, a Cas14 programmable nickase may be multiplexed with an additional programmable nuclease for modification or detection of a target nucleic acid. In some embodiments, the programmable nickase may be a Cas14a programmable nickase, a Cas14b programmable nickase, a Cas14c programmable nickase, a Cas14d programmable nickase, or a Cas14e programmable nickase. In some embodiments, a first programmable nickase (e.g., a Cas14 programmable nickase) may be multiplexed with a second programmable nickase.

In some embodiments, an additional programmable nuclease used in multiplexing is any programmable nuclease. Sometimes, the programmable nuclease is any Cas protein (also referred to as a Cas nuclease herein). In some cases, the programmable nuclease is Cas13. In some embodiments, the Cas13 is Cas13a, Cas13b, Cas13c, Cas13d, or Cas13e. In some cases, the programmable nuclease can be Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 can be also called smCms1, miCms1, obCms1, or suCms1. Sometimes CasZ can be also called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Ping), Enterococcus italicus (Ei), Lactobacillus salivarius (Ls), or Thermus thermophilus (Tt). Any combination of programmable nucleases can be used in multiplexing. In some embodiments, multiplexing of programmable nucleases takes place in one reaction volume. In other embodiments, multiplexing of programmable nucleases takes place in separate reaction volumes in a single device.

### Amplification of a Target Nucleic Acid

Disclosed herein are methods of amplifying a target nucleic acid for detection using any of the methods, reagents, kits or devices described herein. The compositions for amplification of target nucleic acids and methods of use thereof, as described herein, are compatible with the DETECTR assay methods disclosed herein. The compositions for amplification of target nucleic acids and methods of use thereof, as described herein, are compatible with any of the programmable nickases disclosed herein and use of said programmable nickase in a method of detecting a target nucleic acid. A target nucleic acid can be an amplified nucleic acid of interest. The nucleic acid of interest may be any nucleic acid disclosed herein or from any sample as disclosed herein. This amplification can be thermal amplification (e.g., using PCR) or isothermal amplification. This nucleic acid amplification of the sample can improve at least one of sensitivity, specificity, or accuracy of the detection the target nucleic acid. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

The compositions for amplification of target nucleic acids and methods of use thereof, as described herein, are compatible with any of the compositions comprising a programmable nickase and a buffer, which has been developed to improve the function of the programmable nickase and use of said compositions in a method of detecting a target nucleic acid. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence. In some cases, amplification of the target nucleic acid may increase the sensitivity of a detection reaction. In some cases, amplification of the target nucleic acid may increase the specificity of a detection reaction. Amplification of the target nucleic acid may increase the concentration of the target nucleic acid in the sample relative to the concentration of nucleic acids that do not correspond to the target nucleic acid. In some embodiments, amplification of the target nucleic acid may be used to modify the sequence of the target nucleic acid. For example, amplification may be used to insert a PAM sequence into a target nucleic acid that lacks a PAM sequence. In some cases, amplification may be used to increase the homogeneity of a target nucleic acid sequence. For example, amplification may be used to remove a nucleic acid variation that is not of interest in the target nucleic acid sequence.

An amplified target nucleic acid may be present in a DETECTR reaction in an amount relative to an amount of a programmable nickase. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nickase. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nickase. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the programmable nickase. In some embodiments, the programmable nickase is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nickase is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nickase is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

An amplified target nucleic acid may be present in a DETECTR reaction in an amount relative to an amount of a guide nucleic acid. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the guide nucleic acid. In some embodiments, the guide nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

### Kits

Disclosed herein are kits for use to detect or modify a target nucleic acid as disclosed herein using the methods as discuss above. In some embodiments, the kit comprises the programmable nickase system, reagents, and the support medium. The reagents and programmable nickase system can be provided in a reagent chamber or on the support medium. Alternatively, the reagent and programmable nickase system can be placed into the reagent chamber or the support medium by the individual using the kit. Optionally, the kit further comprises a buffer and a dropper. The reagent chamber can be a test well or container. The opening of the reagent chamber can be large enough to accommodate the support medium. The buffer can be provided in a dropper bottle for ease of dispensing. The dropper can be disposable and transfer a fixed volume. The dropper can be used to place a sample into the reagent chamber or on the support medium.

The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, an oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively, or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C. In some cases, the nucleic acid amplification reaction is performed at a temperature of from 20° C. to 45° C., from 25° C. to 40° C., from 30° C. to 40° C., or from 35° C. to 40° C.

In some embodiments, a kit for detecting a target nucleic acid comprising a support medium; a guide nucleic acid targeting a target sequence; a programmable nickase capable of being activated when complexed with the guide nucleic acid and the target sequence; and a reporter nucleic acid comprising a detection moiety, wherein the reporter nucleic acid is capable of being cleaved by the activated nuclease, thereby generating a first detectable signal. Often, the kit further comprises primers for amplifying a target nucleic acid of interest to produce a PAM target nucleic acid.

In some embodiments, a kit for detecting a target nucleic acid comprising a PCR plate; a guide nucleic acid targeting a target sequence; a programmable nickase capable of being activated when complexed with the guide nucleic acid and the target sequence; and a single stranded reporter nucleic acid comprising a detection moiety, wherein the reporter nucleic acid is capable of being cleaved by the activated nuclease, thereby generating a first detectable signal. The wells of the PCR plate can be pre-aliquoted with the guide nucleic acid targeting a target sequence, a programmable nickase capable of being activated when complexed with the guide nucleic acid and the target sequence, and at least one population of a single stranded reporter nucleic acid comprising a detection moiety. A user can thus add the biological sample of interest to a well of the pre-aliquoted PCR plate and measure for the detectable signal with a fluorescent light reader or a visible light reader.

In some embodiments, a kit for modifying a target nucleic acid comprising a support medium; a guide nucleic acid targeting a target sequence; and a programmable nickase capable of being activated when complexed with the guide nucleic acid and the target sequence.

In some embodiments, a kit for modifying a target nucleic acid comprising a PCR plate; a guide nucleic acid targeting a target sequence; and a programmable nickase capable of being activated when complexed with the guide nucleic acid and the target sequence. The wells of the PCR plate can be pre-aliquoted with the guide nucleic acid targeting a target sequence, and a programmable nickase capable of being activated when complexed with the guide nucleic acid and the target sequence. A user can thus add the biological sample of interest to a well of the pre-aliquoted PCR plate.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein the terms “individual,” “subject,” and “patient” are used interchangeably and include any member of the animal kingdom, including humans.

As used herein the term “antibody” refers to, but not limited to, a monoclonal antibody, a synthetic antibody, a polyclonal antibody, a multispecific antibody (including a bi-specific antibody), a human antibody, a humanized antibody, a chimeric antibody, a single-chain Fvs (scFv) (including bi-specific scFvs), a single chain antibody, a Fab fragment, a F(ab′) fragment, a disulfide-linked Fvs (sdFv), or an epitope-binding fragment thereof. In some cases, the antibody is an immunoglobulin molecule or an immunologically active portion of an immunoglobulin molecule. In some instances, an antibody is animal in origin including birds and mammals. Alternately, an antibody is human or a humanized monoclonal antibody.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A method of introducing a break in a target nucleic acid, the method comprising introducing the break by contacting the target nucleic acid with: (a) a first guide nucleic acid comprising a first region that binds to a first programmable nickase having a length of no more than 900 amino acids; and (b) a second guide nucleic acid comprising a first region that binds to a second programmable nickase having a length of no more than 900 amino acids, wherein the first guide nucleic acid comprises a second region that binds to the target nucleic acid and wherein the second guide nucleic acid comprises a second region that binds to the target nucleic acid and wherein the second region of the first guide nucleic acid and the second region of the second guide nucleic acid bind opposing strands of the target nucleic acid. 2. The method of embodiment 1, wherein the first programmable nickase and the second programmable nickase have a length of from 350 to 900 amino acids. 3. The method of any one of embodiments 1-2, wherein the first programmable nickase and the second programmable nickase have a length of from 480 to 550 amino acids. 4. The method of any one of embodiments 1-3, wherein the first programmable nickase and second programmable nickase are a Type V CRISPR/Cas enzyme. 5. The method of embodiment 4, wherein the Type V CRISPR/Cas enzyme comprises three partial RuvC domains. 6. The method of embodiment 5, wherein the three partial RuvC domains are RuvC-I, RuvC-II, and RuvC-III subdomains. 7. The method of any one of embodiments 1-6, wherein the first programmable nickase and the second programmable nickase are a Cas14 protein. 8. The method of embodiment 7, wherein the Cas14 protein is a Cas14a protein, a Cas14b protein, a Cas14c protein, a Cas14d protein, or a Cas14e protein. 9. The method of any one of embodiments 7-8, wherein the Cas14 protein is a Cas14a protein. 10. The method of any one of embodiments 7-8, wherein the Cas14 proteins is a Cas14b protein. 11. The method of any one of embodiments 7-8, wherein the Cas14 protein is a Cas14e protein. 12. The method of any one of embodiments 1-11, wherein the first programmable nickase, the second programmable nickase, or both have at least 80%, at least 85%, at least 90%, at least 92%, at least 95%, at least 97%, or at least 99% sequence identity with any one of SEQ ID NO: 1-SEQ ID NO: 91 or SEQ ID NO: 170. 13. The method of any one of embodiments 1-12, wherein the first programmable nickase, the second programmable nickase, or both are any one of SEQ ID NO: 1-SEQ ID NO: 91 or SEQ ID NO: 170. 14. The method of any one of embodiments 1-13, wherein the first programmable nickase, the second programmable nickase, or both have at least 80%, at least 85%, at least 90%, at least 92%, at least 95%, at least 97%, or at least 99% sequence identity with SEQ ID NO: 1. 15. The method of any one of embodiments 1-14, wherein the first programmable nickase, the second programmable nickase, or both are SEQ ID NO: 1. 16. The method of any one of embodiments 1-13, wherein the first programmable nickase, the second programmable nickase, or both have at least 80%, at least 85%, at least 90%, at least 92%, at least 95%, at least 97%, or at least 99% sequence identity with SEQ ID NO: 10. 17. The method of any one of embodiments 1-13 or 16, wherein the first programmable nickase, the second programmable nickase, or both are SEQ ID NO: 10. 18. The method of any one of embodiments 1-13, wherein the first programmable nickase, the second programmable nickase, or both have at least 80%, at least 85%, at least 90%, at least 92%, at least 95%, at least 97%, or at least 99% sequence identity with SEQ ID NO: 11. 19. The method of any one of embodiments 1-13 or 18, wherein the first programmable nickase, the second programmable nickase, or both are SEQ ID NO: 11. 20. The method of any one of embodiments 1-13, wherein the first programmable nickase, the second programmable nickase, or both have at least 80%, at least 85%, at least 90%, at least 92%, at least 95%, at least 97%, or at least 99% sequence identity with SEQ ID NO: 17. 21. The method of any one of embodiments 1-13 or 20, wherein the first programmable nickase, the second programmable nickase, or both are SEQ ID NO: 17. 22. The method of any one of embodiments 1-13, wherein the first programmable nickase, the second programmable nickase, or both have at least 80%, at least 85%, at least 90%, at least 92%, at least 95%, at least 97%, or at least 99% sequence identity with SEQ ID NO: 33. 23. The method of any one of embodiments 1-13 or 22, wherein the first programmable nickase, the second programmable nickase, or both are SEQ ID NO: 33. 24. The method of any one of embodiments 1-23, wherein the first guide nucleic acid is a first guide RNA. 25. The method of any one of embodiments 1-24, wherein the second guide nucleic acid is a second guide RNA. 26. The method of any one of embodiments 1-25, wherein the first region is a repeat sequence and wherein the second region is a spacer sequence. 27. The method of any one of embodiments 1-26, wherein the first guide nucleic acid and the second guide nucleic acid comprise a crRNA and a tracrRNA. 28. The method of any one of embodiments 1-26, wherein the first guide nucleic acid and the second guide nucleic acid comprise a crRNA and a trancRNA. 29. The method of any one of embodiments 27-28, wherein the crRNA comprises the repeat sequence and the spacer sequence. 30. The method of any one of embodiments 26-29, wherein the repeat sequence hybridizes to a segment of the tracrRNA. 31. The method of any one of embodiments 27-30, wherein the tracrRNA has at least 80%, at least 85%, at least 90%, at least 92%, at least 95%, at least 97%, or at least 99% sequence identity with any one of SEQ ID NO: 98-SEQ ID NO: 99, SEQ ID NO: 101, SEQ ID NO: 103, SEQ ID NO: 105-SEQ ID NO: 151. 32. The method of any one of embodiments 27-31, wherein the tracrRNA is any one of SEQ ID NO: 98-SEQ ID NO: 99, SEQ ID NO: 101, SEQ ID NO: 103, SEQ ID NO: 105-SEQ ID NO: 151. 33. The method of any one of embodiments 27-31, wherein the tracrRNA has at least 80%, at least 85%, at least 90%, at least 92%, at least 95%, at least 97%, or at least 99% sequence identity with SEQ ID NO: 99 34. The method of any one of embodiments 27-31 or 33, wherein the tracrRNA is SEQ ID NO: 99. 35. The method of any one of embodiments 27-31, wherein the tracrRNA has at least 80%, at least 85%, at least 90%, at least 92%, at least 95%, at least 97%, or at least 99% sequence identity with SEQ ID NO: 101. 36. The method of any one of embodiments 27-31 or 35, wherein the tracrRNA is SEQ ID NO: 101. 37. The method of any one of embodiments 27-31, wherein the tracrRNA has at least 80%, at least 85%, at least 90%, at least 92%, at least 95%, at least 97%, or at least 99% sequence identity with SEQ ID NO: 103. 38. The method of any one of embodiments 27-31 or 37, wherein the tracrRNA is SEQ ID NO: 103. 39. The method of any one of embodiments 27-31, wherein the tracrRNA has at least 80%, at least 85%, at least 90%, at least 92%, at least 95%, at least 97%, or at least 99% sequence identity with SEQ ID NO: 119. 40. The method of any one of embodiments 27-31 or 39, wherein the tracrRNA is SEQ ID NO: 119. 41. The method of any one of embodiments 1-40, wherein the first programmable nickase and the second programmable nickase exhibit 2-fold greater nicking activity as compared to double stranded cleavage activity. 42. The method of any one of embodiments 1-41, wherein the first programmable nickase and the second programmable nickase nick the target nucleic acid at two different sites. 43. The method of any one of embodiments 1-42, wherein the target nucleic acid comprises double stranded DNA. 44. The method of embodiment 43, wherein the two different sites are on opposing strands of the double stranded DNA. 45. The method of any one of embodiments 1-44, wherein the target nucleic acid comprises a mutated sequence or a sequence is associated with a disease. 46. The method of embodiment 45, wherein the disease is cancer. 47. The method of any one of embodiments 1-46, wherein the method comprises administering the first programmable nickase and the second programmable nickase to a subject in need thereof 48. The method of embodiment 45, wherein the mutated sequence is removed after the first programmable nickase and the second programmable nickase nick the target nucleic acid. 49. The method of any one of embodiments 1-48, wherein the first programmable nickase and the second programmable nickase are the same. 50. The method of any one of embodiments 1-49, wherein the first programmable nickase and the second programmable nickase are different. 1. A method of introducing a break in a target nucleic acid, the method comprising introducing the break by contacting the target nucleic acid with: (a) a first guide RNA comprising a first region that binds to a first programmable nickase; and (b) a second guide RNA comprising a first region that binds to a second programmable nickase, wherein the first guide RNA comprises a second region that binds to the target nucleic acid and wherein the second guide RNA comprises a second region that binds to the target nucleic acid and wherein the second region of the first guide RNA and the second region of the second guide RNA bind opposing strands of the target nucleic acid. 2. The method of embodiment 1, wherein the first programmable nickase and the second programmable nickase nick the target nucleic acid at two different sites. 3. The method of embodiment 1, wherein the target nucleic acid comprises double stranded DNA. 4. The method of embodiment 3, wherein the two different sites are on opposing strands of the double stranded DNA. 5. The method of embodiment 1, wherein the target nucleic acid comprises a mutated sequence or a sequence is associated with a disease. 6. The method of embodiment 5, wherein the disease is cancer. 7. The method of embodiment 1, wherein the method comprising administering the first programmable nickase and the second programmable nickase to a subject in need thereof 8. The method of embodiment 5, wherein the mutated sequence is removed after the first programmable nuclease and the second programmable nuclease nick the target nucleic acid. 9. The method of embodiment 1, wherein the first programmable nickase and the second programmable nickase comprise a Cas14 protein. 10. A method of detecting a target nucleic acid in a sample, the method comprising contacting the sample with (a) a programmable nuclease; (b) a guide RNA comprising a first region that binds to the programmable nickase and a second region that binds to the target nucleic acid; and (c) a labeled, single stranded DNA reporter that does not bind the guide RNA; cleaving the labeled single stranded DNA reporter to release a detectable label; and detecting the target nucleic acid by measuring a signal from the detectable label. 11. The method of embodiment 10, wherein the target nucleic acid is single stranded DNA. 12. The method of embodiment 10, wherein the programmable nickase comprises a Cas 14 protein. 13. The method of embodiment 1 or 10, wherein the target nucleic acid is in a sample. 14. The method of embodiment 13, wherein the sample comprises a phosphate buffer, a Tris buffer, or a HEPES buffer. 15. The method of embodiment 13, wherein the sample comprises a pH of 7 to 9. 16. The method of embodiment 13, wherein the sample comprises a pH of 7.5 to 8. 17. The method of embodiment 13, wherein the sample comprises a salt concentration of 25 nM to 200 mM. 18. The method of embodiment 10, wherein the single stranded DNA reporter comprises an ssDNA-fluorescence quenching DNA reporter. 19. The method of embodiment 18, wherein the ssDNA-fluorescence quenching DNA reporter is a universal ssDNA-fluorescence quenching DNA reporter. 20. The method of embodiment 1 or 10, wherein the programmable nickase exhibits PAM-independent nicking and cleaving. 21. The method of embodiment 9 or 12, wherein the Cas14 protein comprises a Cas14e protein. 22. The method of embodiment 9 or 12, wherein the Cas14 protein comprises from 400 to 800 amino acid residues. 23. A composition comprising a programmable nickase and a guide RNA comprising a first region that binds the programmable nickase and a second region that binds a target nucleic acid. 24. The composition of embodiment 23, wherein the target nucleic acid comprises single stranded DNA or double stranded DNA. 25. The composition of embodiment 23, wherein the programmable nickase exhibits PAM-independent nicking and cleaving. 26. The composition of embodiment 23, wherein the programmable nickase nicks a single strand of the double stranded DNA. 27. The composition of embodiment 23, wherein the programmable nickase cleaves single stranded DNA. 28. The composition of embodiment 23, wherein the programmable nickase comprises a Cas14 protein. 29. The composition of embodiment 28, wherein the Cas14 protein comprises a Cas14e protein. 30. The composition of embodiment 29, wherein the Cas14 protein comprises from 400 to 800 amino acid residues.

### EXAMPLES

The following examples are included to further describe some aspects of the present disclosure, and should not be used to limit the scope of the invention.

### Example 1

### Cas14e is a Programmable Nickase

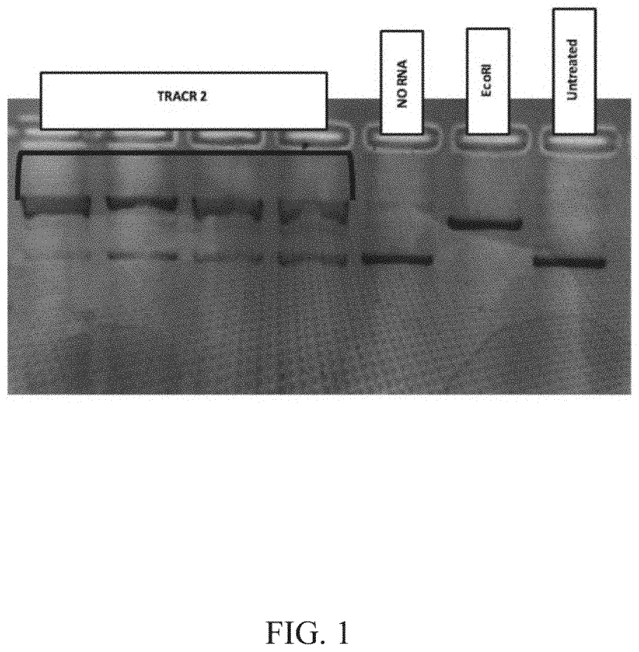

The present example shows that Cas14e is a programmable nickase. FIG. 1 shows a gel illustrating nicking of dsDNA by four different Cas14e proteins. In the gel, four different Cas14e proteins were independently added to the first four lanes along with a guide RNA (TRACR2), which formed a complex with each Cas14e protein. When the guide RNA was complexed with the Cas14e protein and when this complex bound to its target nucleic acid, the nickase activity of the Cas14e proteins was activated. This is shown in the first four lanes of the gel by the resulting two bands, in which the upper band is the nicked target dsDNA. The fifth lane was a control lane comprising a Cas14e protein, but no guide RNA, in which the target dsDNA remained intact. The sixth lane shows cleavage of dsDNA by a restriction enzyme, EcoRI, which generated a double strand break in the target dsDNA. The seventh lane shows untreated target dsDNA (e.g., no programmable nickase, guide RNA, or restriction enzyme). Therefore, Cas14e is a programmable nickase.

### Example 2

### Introducing Strand Breaks in dsDNA Using Programmable Nickases

The present example describes introducing breaks in a dsDNA using programmable nickases (e.g., a Cas14 such as Cas14e). Two programmable nickases, such as two Cas14e targeting a two different strands of a dsDNA, are co-delivered. The first Cas14e protein is bound to a first guide RNA targeting first region of dsDNA and the second Cas14e protein is bound to a second guide RNA targeting a second region that is on an opposing strand of the dsDNA. Opposing target DNA strands are nicked by the Cas14e proteins and two breaks in the dsDNA are generated. These two strand breaks are repaired and rejoined by non-homologous end joining (NHEJ) or homology directed repair (HDR). Thus, two programmable nickases are combined to selectively edit sequences of target nucleic acid molecules.

### Example 3

### Tuning Cas14e Programmable Nickase Activity with Buffer, pH, and Temperature

This example describes tuning Cas14e programmable nickase activity with buffer, pH, and temperature in a DETECTR assay. Cas14e programmable nickases were incubated with a sample containing target ssDNA, which activated the Cas14e programmable nickases to indiscriminately cleave an ssDNA-FQ reporter. Cleaved ssDNA-FW reporters released a detectable label, which was measured by fluorescence readings. This DETECTR assay was run under various buffer, pH, and temperature conditions, with on-target guide RNA and an off-target guide RNA control.

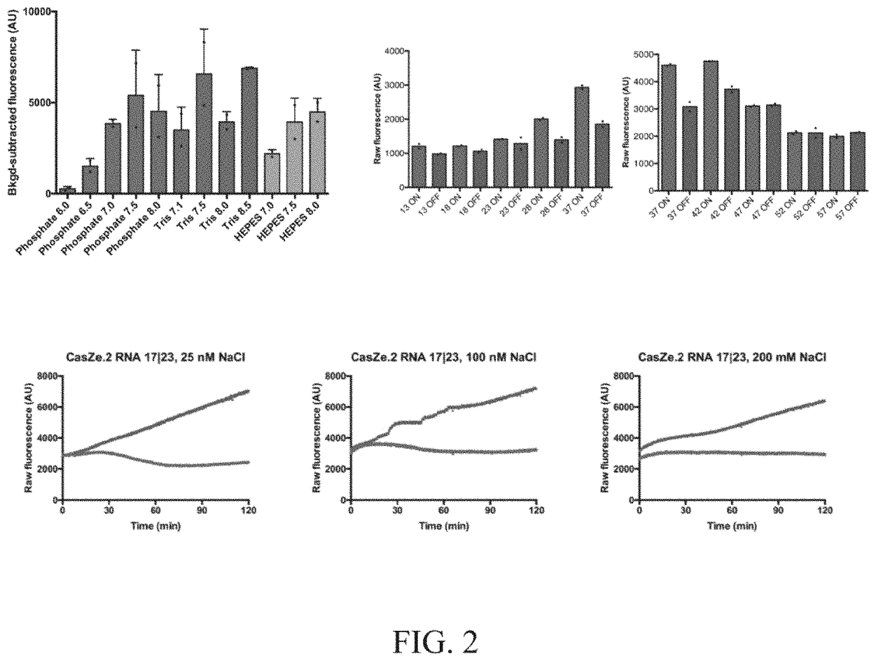

FIG. 2 shows the effect of salt, buffer, and temperature on an ssDNA DETECTR reaction using Cas14e. At the top left is a bar graph showing various buffer conditions and pH levels on the x-axis and the background subtracted fluorescence on the y-axis. Fluorescence indicates cleavage of a reporter. Greater fluorescence indicates more activity. At the top middle and top right are graphs showing temperatures on the x-axis (“ON” indicates the target ssDNA that can hybridize to the guide RNA was added; “OFF” indicates off-target ssDNA that does not hybridize to the guide RNA was added) versus raw fluorescence on the y-axis. Fluorescence indicates cleavage of a reporter.

### Example 4

### Cas14e Trans-Cleavage Activity Independent of Salt Concentration

This example shows Cas14e trans-cleavage activity is independent of salt concentration in a DETECTR assay. Cas14e proteins were incubated with a sample containing target ssDNA, which activated the Cas14e trans-cleavage activity to indiscriminately cleave an ssDNA-FQ reporter. Cleaved ssDNA-FQ reporters released a detectable label, which was measured by fluorescence readings. This DETECTR assay was run under various salt concentrations including 25 nM NaCl, 100 nM NaCl, and 200 mM NaCl, in the presence of target ssDNA and in the presence of off-target ssDNA.

The bottom three line graphs of FIG. 2 show fluorescence over time in various salt conditions (25 nM NaCl, 100 nM NaCl, and 200 mM NaCl from left to right). Fluorescence indicates cleavage of a reporter. The higher line, with increasing fluorescence over time, shows cleavage of reporters by Cas14e proteins complexed with guide RNAs in the presence of target ssDNA. The lower line shows background cleavage of reporters by Cas14e proteins complexed with guide RNAs in the presence of off-target ssDNA. Surprisingly, it was discovered that the Cas14e programmable nickases were functional even at very high salt concentrations, suggesting that these nickases function independent of salt concentration.

### Example 5

### ssDNA-FQ Reporter Sequence Independent Cas14e

This example shows ssDNA-FQ reporter sequence independent activity of Cas14e proteins in a DETECTR assay. Cas14e proteins were incubated with a sample containing target ssDNA, which activated the Cas14e proteins to indiscriminately cleave one of three different homopolymers of ssDNA-FQ reporter (T12, A12, or C12). Cleaved ssDNA-FQ reporters released a detectable label, which was measured by fluorescence readings.

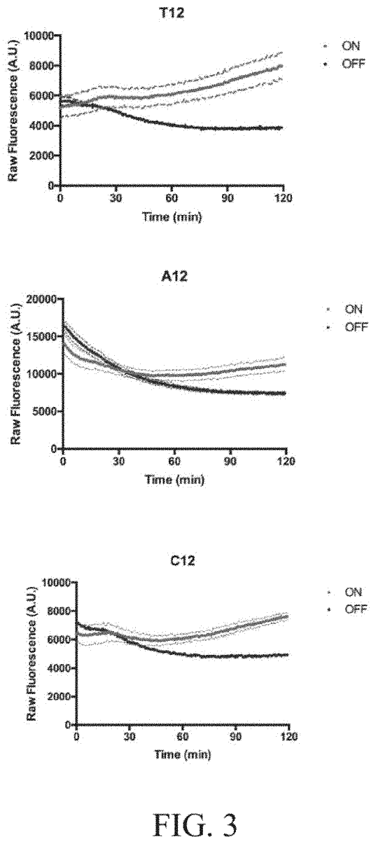

FIG. 3 shows three graphs, which from left to right assess cleavage of homopolymer fluorescence-quenching (FQ) reporters. Fluorescence indicates cleavage of a reporter. Greater fluorescence indicates more activity. The left most graph uses a T12 (12 thymine residues) ssDNA-FQ reporter, the middle graph uses an A12 (12 adenine residues) ssDNA-FQ reporter, and the right most graph uses a C12 (12 cytosine residues) ssDNA-FQ reporter. In each graph, the top lines show cleavage of reporters by Cas14e proteins complexed with guide RNAs in the presence of target ssDNA and the bottom lines show cleavage of reporters by Cas14e proteins complexed with guide RNAs in the presence of off-target ssDNA.

### Example 6

### Cas14e PAM-Insensitivity

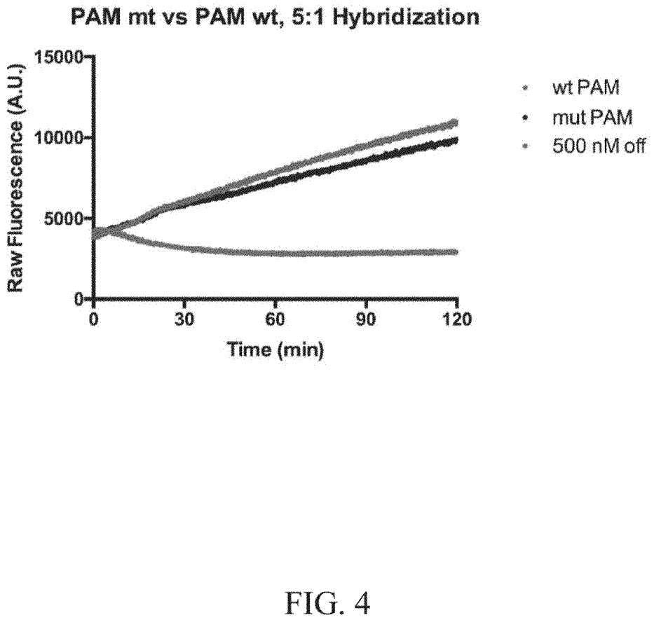

This example describes Cas14e PAM-insensitivity. FIG. 4 shows a graph of fluorescence over time for three DETECTR reactions using Cas14e proteins coupled to a guide RNA to detect target dsDNA. Fluorescence indicates cleavage of a reporter. Greater fluorescence indicates more activity. The top most line shows cleavage of reporters in the presence of a target dsDNA having a wild type (wt) PAM. The line immediately below the top most line shows cleavage of reporters in the presence of a target dsDNA having a mutant (mut) PAM. In some embodiments, the mutant PAM differs from the native PAM by a single nucleotide. In some embodiments, the mutant PAM differs from the native PAM by multiple nucleotides. In some embodiments, the mutant PAM is shorter than the native PAM. In some embodiments, the mutant PAM is longer than the native PAM. The lowest line shows cleavage of reporters in the presence of 500 nM of off-target dsDNA. The results showed that Cas14e can detect a target dsDNA without having PAM restrictions.

### Example 7

### Cas14a and Cas14b Nicking and Cleavage Activities

This example describes an assay measuring nicking and cleavage activity for a variety of programmable nickase complexed with a variety of guide nucleic acids. The effect of varying the tracr sequence and Cas14 sequence on nicking and cleaving of target nucleic acids was tested by separately measuring the activities of four different Cas14 orthologs of SEQ ID NO: 1, SEQ ID NO: 10, SEQ ID NO: 11 and SEQ ID NO: 17 complexed with distinct guide nucleic acids. The Cas14 programmable nickase were complexed with a crRNA sequence targeting SEQ ID NO: 96 and variety of tracr sequences shown in TABLE 3 (below). The sequences of the Cas14, gRNA pairs used in each assay are shown in TABLE 3.

TABLE 3
Guide Nucleic Acid, Target Nucleic Acid and Programmable
Nickase Sequences

| Species | SEQ ID NO | Sequence |
|---|---|---|
| Cas14a.3 | SEQ ID NO: 1 | MEVQKTVMKTLSLRILRPLYSQEIEKEIKEEK |
| Programmable | | ERRKQAGGTGELDGGFYKKLEKKHSEMFSF |
| Nickase | | DRLNLLLNQLQREIAKVYNHAISELYIATIAQ |
| | | GNKSNKHYISSIVYNRAYGYFYNAYIALGICS |
| | | KVEANFRSNELLTQQSALPTAKSDNFPIVLHK |
| | | QKGAEGEDGGFRISTEGSDLIFEIPIPFYEYNG |
| | | ENRKEPYKWVKKGGQKPVLKLILSTFRRQRN |
| | | KGWAKDEGTDAEIRKVTEGKYQVSQIEINRG |
| | | KKLGEHQKWFANFSIEQPIYERKPNRSIVGGL |
| | | DVGIRSPLVCAINNSFSRYSVDSNDVFKFSKQ |
| | | VFAFRRRLLSKNSLKRKGHGAAHKLEPITEM |
| | | TEKNDKFRKKIIERWAKEVTNFFVKNQVGIV |
| | | QIEDLSTMKDREDHFFNQYLRGFWPYYQMQ |
| | | TLIENKLKEYGIEVKRVQAKYTSQLCSNPNC |
| | | RYWNNYFNFEYRKVNKFPKFKCEKCNLEISA |
| | | DYNAARNLSTPDIEKFVAKATKGINLPEK |
| DNA encoding | SEQ ID NO: 92 | ATGGAAGTGCAGAAAACCGTGATGAAAAC |
| Cas 14a.3 | | CCTGAGCCTGCGGATCCTGCGGCCTCTGTA |
| Programmable | | CTCTCAAGAGATCGAGAAAGAGATCAAAG |
| Nickase | | AGGAAAAAGAGCGCCGGAAGCAGGCTGGC |
| | | GGAACAGGCGAACTTGATGGCGGCTTCTAC |
| | | AAGAAGCTGGAAAAGAAACACAGCGAGAT |
| | | GTTCAGCTTCGACCGGCTGAACCTGCTGCT |
| | | GAATCAGCTGCAGCGGGAAATCGCCAAGG |
| | | TGTACAACCACGCCATCAGCGAGCTGTATA |
| | | TCGCCACAATCGCCCAGGGCAACAAGAGC |
| | | AACAAGCACTACATCAGCAGCATCGTGTAC |
| | | AACAGAGCCTACGGCTACTTCTACAACGCC |
| | | TATATCGCCCTGGGCATCTGCAGCAAGGTG |
| | | GAAGCCAACTTCAGAAGCAACGAGCTGCT |
| | | GACCCAGCAGAGCGCACTGCCTACAGCCA |
| | | AGAGCGACAACTTCCCCATCGTGCTGCACA |
| | | AGCAGAAAGGCGCCGAAGGCGAGGATGGC |
| | | GGATTCAGAATCAGCACCGAGGGCAGCGA |
| | | CCTGATCTTCGAGATCCCCATTCCATTCTAC |
| | | GAGTACAACGGCGAGAACCGGAAAGAACC |
| | | CTACAAATGGGTCAAGAAAGGCGGGCAGA |
| | | AACCAGTGCTGAAGCTGATCCTGAGCACCT |
| | | TCCGGCGGCAGAGAAACAAAGGCTGGGCC |
| | | AAAGATGAGGGCACCGACGCCGAGATCAG |
| | | AAAAGTGACAGAGGGCAAGTACCAGGTGT |
| | | CCCAGATCGAGATCAACCGGGGCAAGAAA |
| | | CTGGGCGAGCACCAGAAGTGGTTCGCCAAT |
| | | TTCAGCATCGAGCAGCCCATCTACGAGCGG |
| | | AAGCCCAACAGATCTATCGTCGGCGGCCTG |
| | | GACGTGGGCATTAGATCTCCACTCGTGTGC |
| | | GCCATCAACAACAGCTTCTCCCGGTACAGC |
| | | GTGGACAGCAACGACGTGTTCAAGTTCAGC |
| | | AAACAGGTGTTCGCCTTCAGACGGCGGCTG |
| | | CTGAGCAAGAACAGCCTGAAGAGAAAAGG |
| | | CCACGGCGCTGCCCACAAGCTGGAACCTAT |
| | | CACCGAGATGACCGAGAAGAACGACAAGT |
| | | TCCGGAAGAAAATCATCGAGCGCTGGGCT |
| | | AAAGAAGTGACCAACTTCTTCGTGAAGAAT |
| | | CAAGTGGGCATTGTGCAGATCGAGGACCTG |
| | | TCCACCATGAAGGACAGAGAGGACCACTT |
| | | CTTCAACCAGTACCTGAGAGGCTTCTGGCC |
| | | CTACTACCAGATGCAGACCCTGATCGAGAA |
| | | CAAGCTGAAAGAATACGGCATCGAAGTGA |
| | | AGCGCGTGCAGGCCAAGTACACCAGCCAG |
| | | CTGTGCAGCAACCCCAACTGCCGGTACTGG |
| | | AACAACTACTTCAACTTCGAGTATCGGAAA |
| | | GTGAACAAGTTCCCCAAGTTCAAGTGCGAG |
| | | AAGTGCAACCTGGAAATCAGCGCCGACTA |
| | | CAATGCCGCCAGAAACCTGAGCACACCCG |
| | | ACATCGAGAAGTTCGTGGCCAAGGCCACC |
| | | AAGGGCATCAACCTGCCTGAGAAGTGA |
| Cas14b.3 | SEQ ID NO: 10 | MEESIITGVKFKLRIDKETTKKLNEYFDEYGK |
| Programmable | | AINFAVKIIQKELADDRFAGKAKLDQNKNPIL |
| Nickase | | DENGKKIYEFPDEFCSCGKQVNKYVNNKPFC |
| | | QECYKIRFTENGIRKRMYSAKGRKAEHKINIL |
| | | NSTNKISKTHFNYAIREAFILDKSIKKQRKKR |
| | | NERLRESKKRLQQFIDMRDGKREICPTIKGQK |
| | | VDRFIHPSWITKDKKLEDFRGYTLSIINSKIKIL |
| | | DRNIKREEKSLKEKGQIIFKAKRLMLDKSIRF |
| | | VGDRKVLFTISKTLPKEYELDLPSKEKRLNW |
| | | LKEKIEIIKNQKPKYAYLLRKNIESEKKPNYE |
| | | YYLQYTLEIKPELKDFYDGAIGIDRGINHIAV |
| | | CTFISNDGKVTPPKFFSSGEILRLKNLQKERD |
| | | RFLLRKHNKNRKKGNMRVIENKINLILHRYS |
| | | KQIVDMAKKLNASIVFEELGRIGKSRTKMKK |
| | | SQRYKLSLFIFKKLSDLVDYKSRREGIRVTYV |
| | | PPEYTSKECSHCGEKVNTQRPFNGNYSLFKC |
| | | NKCGIQLNSDYNASINIAKKGLKIPNST |
| DNA Encoding | SEQ ID NO: 93 | ATGGAAGAGTCCATCATCACCGGCGTGAA |
| Cas14b.3 | | GTTCAAGCTGCGGATCGACAAAGAAACCA |
| Programmable | | CCAAGAAGCTGAACGAGTACTTCGACGAG |
| Nickase | | TACGGCAAGGCCATCAACTTCGCCGTGAAG |
| | | ATCATCCAGAAAGAGCTGGCCGACGACAG |
| | | ATTCGCCGGAAAGGCCAAGCTGGACCAGA |
| | | ACAAGAACCCCATCCTGGACGAGAATGGC |
| | | AAGAAGATCTACGAGTTCCCCGACGAGTTC |
| | | TGCAGCTGCGGCAAGCAAGTGAACAAATA |
| | | CGTGAACAACAAGCCCTTCTGCCAAGAGTG |
| | | CTACAAGATCCGGTTCACCGAGAACGGCAT |
| | | CCGGAAGAGAATGTACAGCGCCAAGGGCA |
| | | GAAAGGCCGAGCACAAGATCAACATCCTG |
| | | AACAGCACCAACAAGATCAGCAAGACCCA |
| | | CTTCAACTACGCCATCAGAGAGGCCTTCAT |
| | | CCTGGATAAGAGCATCAAGAAGCAGCGGA |
| | | AGAAGCGCAACGAGCGGCTGAGAGAGAGC |
| | | AAGAAGAGACTGCAGCAGTTCATCGACAT |
| | | GCGCGACGGCAAGAGAGAGATCTGCCCTA |
| | | CCATCAAGGGCCAGAAGGTGGACCGGTTC |
| | | ATTCACCCCAGCTGGATCACCAAGGACAAG |
| | | AAGCTCGAGGACTTCCGGGGCTACACCCTG |
| | | AGCATCATCAACAGCAAGATCAAGATTCTG |
| | | GACCGGAACATCAAGCGGGAAGAGAAGTC |
| | | CCTGAAAGAGAAGGGGCAGATCATCTTCA |
| | | AGGCCAAGAGACTGATGCTGGACAAGTCC |
| | | ATCAGATTCGTGGGCGACAGAAAGGTGCT |
| | | GTTTACCATCTCCAAGACACTGCCCAAAGA |
| | | GTACGAGCTGGACCTGCCTAGCAAAGAGA |
| | | AGCGGCTGAACTGGCTGAAAGAAAAGATC |
| | | GAGATCATCAAGAACCAGAAGCCGAAGTA |
| | | CGCCTACCTGCTGCGGAAGAACATCGAGA |
| | | GCGAGAAGAAGCCCAATTACGAGTACTAC |
| | | CTGCAGTACACCCTGGAAATCAAGCCCGAG |
| | | CTGAAGGACTTCTACGACGGCGCCATCGGC |
| | | ATCGACAGAGGCATCAATCACATTGCCGTG |
| | | TGCACCTTCATCAGCAACGACGGCAAAGTG |
| | | ACCCCTCCTAAGTTCTTCAGCAGCGGCGAG |
| | | ATCCTGAGACTGAAGAACCTGCAGAAAGA |
| | | ACGCGACCGGTTCCTGCTGAGAAAGCACA |
| | | ACAAGAATCGGAAGAAAGGCAACATGCGC |
| | | GTGATCGAGAACAAGATTAACCTGATCCTG |
| | | CACCGGTACAGCAAGCAGATCGTGGACAT |
| | | GGCCAAAAAGCTGAATGCCAGCATCGTGTT |
| | | CGAGGAACTGGGCAGAATCGGCAAGAGCC |
| | | GGACCAAGATGAAGAAGTCCCAGCGGTAC |
| | | AAGCTGAGCCTGTTCATCTTTAAGAAACTG |
| | | AGCGACCTGGTGGACTACAAGTCTCGGAG |
| | | AGAAGGCATCAGAGTGACCTACGTGCCAC |
| | | CAGAGTACACCAGCAAAGAGTGCTCTCACT |
| | | GCGGAGAGAAAGTGAACACCCAGCGGCCT |
| | | TTCAACGGCAACTACTCCCTGTTCAAGTGC |
| | | AACAAGTGCGGCATCCAGCTGAACAGCGA |
| | | CTACAACGCCAGCATCAATATCGCCAAGAA |
| | | GGGCCTGAAGATCCCCAACTCCACCTGA |
| Cas14b.4 | SEQ ID NO: 11 | MPKQDLVTTGIKFKLDVDKETRKKLDDYFD |
| Programmable | | EYGKAINFAVKIIQKNLKEDRFAGKIALGEDK |
| Nickase | | KPLLDKDGKKIYNYPNESCSCGNQVRRYVN |
| | | AKPFCVDCYKLKFTENGIRKRMYSARGRKA |
| | | DSDINIKNSTNKISKTHFNYAIREGFILDKSLK |
| | | KQRSKRIKKLLELKRKLQEFIDIRQGQMVLCP |
| | | KIKNQRVDKFIHPSWLKRDKKLEEFRGYSLS |
| | | VVEGKIKIFNRNILREEDSLRQRGHVNFKANR |
| | | IMLDKSVRFLDGGKVNFNLNKGLPKEYLLDL |
| | | PKKENKLSWLNEKISLIKLQKPKYAYLLRRE |
| | | GSFFIQYTIENVPKTFSDYLGAIGIDRGISHIAV |
| | | CTFVSKNGVNKAPVFFSSGEILKLKSLQKQR |
| | | DLFLRGKHNKIRKKSNMRNIDNKINLILHKYS |
| | | RNIVNLAKSEKAFIVFEKLEKIKKSRFKMSKS |
| | | LQYKLSQFTFKKLSDLVEYKAKIEGIKVDYV |
| | | PPEYTSKECSHCGEKVDTQRPFNGNSSLFKC |
| | | NKCRVQLNADYNASINIAKKSLNISN |
| DNA Encoding | SEQ ID NO: 94 | ATGCCCAAGCAGGATCTGGTCACCACCGGC |
| Cas14b.4 | | ATCAAGTTCAAGCTGGACGTGGACAAAGA |
| Programmable | | GACACGGAAGAAACTGGACGACTACTTCG |
| Nickase | | ACGAGTACGGCAAGGCCATCAACTTCGCCG |
| | | TGAAGATCATCCAGAAGAACCTGAAAGAG |
| | | GACCGCTTCGCCGGCAAGATTGCCCTGGGC |
| | | GAAGATAAGAAGCCCCTGCTGGACAAGGA |
| | | CGGCAAGAAGATCTACAACTACCCCAACG |
| | | AGAGCTGCTCCTGCGGCAATCAAGTGCGGA |
| | | GATACGTGAACGCCAAGCCTTTCTGCGTGG |
| | | ACTGCTACAAGCTGAAGTTCACCGAGAACG |
| | | GCATCCGGAAGCGGATGTACTCTGCCAGAG |
| | | GAAGAAAGGCCGACAGCGACATCAACATC |
| | | AAGAACAGCACCAACAAGATCAGCAAGAC |
| | | CCACTTCAACTACGCCATCAGAGAGGGCTT |
| | | CATCCTGGACAAGAGCCTGAAGAAGCAGC |
| | | GGAGCAAGCGGATCAAGAAGCTGCTGGAA |
| | | CTGAAGCGGAAGCTGCAAGAGTTCATCGA |
| | | CATCCGGCAGGGCCAGATGGTGCTGTGCCC |
| | | CAAGATCAAGAACCAGAGAGTGGACAAGT |
| | | TCATTCACCCCAGCTGGCTGAAGAGAGACA |
| | | AAAAGCTGGAAGAATTCCGGGGCTACAGC |
| | | CTGAGCGTGGTGGAAGGCAAGATTAAGAT |
| | | CTTCAACCGGAACATCCTGCGCGAAGAGG |
| | | ACTCCCTGAGACAGAGGGGCCACGTGAAC |
| | | TTTAAGGCCAACCGGATCATGCTGGATAAG |
| | | AGCGTGCGGTTCCTGGACGGCGGCAAAGT |
| | | GAATTTCAACCTGAACAAGGGCCTGCCGAA |
| | | AGAGTACCTGCTGGATCTGCCCAAGAAAG |
| | | AGAACAAGCTGTCCTGGCTGAACGAAAAG |
| | | ATCAGCCTGATCAAGCTGCAGAAGCCTAAG |
| | | TACGCCTACCTGCTGAGAAGAGAGGGCAG |
| | | CTTTTTCATCCAGTACACCATCGAGAACGT |
| | | GCCCAAGACCTTCAGCGATTACCTGGGCGC |
| | | CATCGGCATCGACAGAGGCATCTCTCACAT |
| | | TGCCGTGTGCACCTTCGTGTCCAAGAACGG |
| | | CGTGAACAAGGCCCCTGTGTTCTTCAGCTC |
| | | TGGCGAGATCCTGAAGCTGAAAAGCCTGC |
| | | AGAAACAGAGGGACCTGTTCCTGCGGGGC |
| | | AAGCACAACAAAATCCGGAAGAAAAGCAA |
| | | CATGCGGAACATCGACAACAAGATTAACCT |
| | | GATCCTGCACAAGTACAGCCGCAACATCGT |
| | | GAACCTGGCCAAGAGCGAGAAGGCCTTTA |
| | | TCGTGTTCGAGAAGCTCGAGAAGATCAAA |
| | | AAGTCCCGGTTCAAGATGAGCAAGTCCCTG |
| | | CAGTATAAGCTGAGCCAGTTCACCTTCAAG |
| | | AAACTGAGCGACCTGGTCGAGTACAAGGC |
| | | CAAGATCGAGGGCATCAAGGTGGACTACG |
| | | TGCCACCTGAGTACACCAGCAAAGAGTGCT |
| | | CTCACTGCGGCGAGAAGGTGGACACCCAG |
| | | AGGCCTTTCAACGGCAACAGCAGCCTGTTC |
| | | AAGTGTAACAAGTGCCGGGTGCAGCTGAA |
| | | CGCCGACTACAACGCCAGCATCAATATCGC |
| | | CAAGAAGTCCCTGAACATCAGCAACAACT |
| | | GA |
| Cas14.b10 | SEQ ID NO: 17 | MEKNNSEQTSITTGIKFKLKLDKETKEKLNN |
| Programmable | | YFDEYGKAINFAVRIIQMQLNDDRLAGKYKR |
| Nickase | | DEKGKPILGEDGKKILEIPNDFCSCGNQVNHY |
| | | VNGVSFCQECYKKRFSENGIRKRMYSAKGR |
| | | KAEQDINIKNSTNKISKTHFNYAIREAFNLDK |
| | | SIKKQREKRFKKLKDMKRKLQEFLEIRDGKR |
| | | VICPKIEKQKVERYIHPSWINKEKKLEEFRGY |
| | | SLSIVNSKIKSFDRNIQREEKSLKEKGQINFKA |
| | | QRLMLDKSVKFLKDNKVSFTISKELPKTFELD |
| | | LPKKEKKLNWLNEKLEIIKNQKPKYAYLLRK |
| | | ENNIFLQYTLDSIPEIHSEYSGAVGIDRGVSHI |
| | | AVYTFLDKDGKNERPFFLSSSGILRLKNLQKE |
| | | RDKFLRKKHNKIRKKGNMRNIEQKINLILHE |
| | | YSKQIVNFAKDKNAFIVFELLEKPKKSRERMS |
| | | KKIQYKLSQFTFKKLSDLVDYKAKREGIKVIY |
| | | VEPAYTSKDCSHCGERVNTQRPFNGNFSLFK |
| | | CNKCGIVLNSDYNASLNIARKGLNISAN |
| DNA Encoding | SEQ ID NO: 95 | ATGGAAAAGAACAACAGCGAGCAGACCAG |
| Cas14b.10 | | CATCACCACCGGCATCAAGTTCAAGCTGAA |
| Programmable | | GCTGGACAAAGAGACAAAAGAGAAGCTGA |
| Nickase | | ACAACTACTTCGACGAGTACGGCAAGGCC |
| | | ATCAACTTCGCCGTGCGGATCATCCAGATG |
| | | CAGCTGAACGACGATAGACTGGCCGGCAA |
| | | GTACAAGCGGGACGAGAAGGGAAAGCCTA |
| | | TCCTGGGCGAAGATGGCAAGAAGATCCTG |
| | | GAAATCCCCAACGACTTCTGCAGCTGCGGC |
| | | AATCAAGTGAACCACTACGTGAACGGCGT |
| | | GTCCTTCTGCCAAGAGTGCTACAAGAAGCG |
| | | GTTCAGCGAGAACGGCATCCGGAAGAGAA |
| | | TGTACAGCGCCAAGGGCAGAAAGGCCGAG |
| | | CAGGACATCAACATCAAGAACAGCACCAA |
| | | CAAGATCAGCAAGACCCACTTCAACTACGC |
| | | CATCAGAGAGGCCTTCAACCTGGACAAGA |
| | | GCATCAAGAAGCAGAGGGAAAAGCGCTTC |
| | | AAGAAACTGAAGGACATGAAGCGGAAGCT |
| | | GCAAGAGTTCCTCGAGATCCGCGACGGCA |
| | | AGAGAGTGATCTGCCCCAAGATCGAGAAG |
| | | CAGAAGGTGGAACGGTACATTCACCCCAG |
| | | CTGGATCAACAAAGAGAAGAAGCTGGAAG |
| | | AATTCCGGGGCTACAGCCTGAGCATCGTGA |
| | | ACAGCAAGATCAAGAGCTTCGACCGGAAC |
| | | ATCCAGCGCGAGGAAAAGAGCCTGAAAGA |
| | | GAAGGGCCAGATCAACTTCAAGGCCCAGC |
| | | GGCTGATGCTGGATAAGAGCGTGAAGTTCC |
| | | TCAAGGACAACAAGGTGTCCTTCACCATCA |
| | | GCAAAGAGCTGCCCAAGACCTTCGAGCTG |
| | | GACCTGCCTAAGAAAGAGAAAAAACTGAA |
| | | CTGGCTGAACGAGAAGCTCGAGATCATTAA |
| | | GAACCAGAAGCCGAAGTACGCCTACCTGCT |
| | | GCGCAAAGAGAACAACATCTTCCTGCAGTA |
| | | CACCCTGGACAGCATCCCCGAGATCCACAG |
| | | CGAATATTCTGGCGCCGTGGGCATCGATAG |
| | | AGGCGTGTCACATATCGCCGTGTACACCTT |
| | | CCTGGATAAGGACGGAAAGAACGAGCGGC |
| | | CATTCTTCCTGAGCAGCAGCGGCATCCTGC |
| | | GGCTGAAGAACCTGCAGAAAGAGCGGGAC |
| | | AAGTTCCTGCGGAAGAAGCACAACAAAAT |
| | | CCGGAAAAAGGGCAACATGCGGAACATCG |
| | | AGCAGAAGATCAACCTGATCCTGCACGAGT |
| | | ACTCCAAGCAGATCGTGAACTTTGCCAAGG |
| | | ACAAGAACGCCTTCATCGTGTTCGAGCTGC |
| | | TGGAAAAGCCCAAGAAAAGCCGCGAGCGG |
| | | ATGAGCAAGAAAATCCAGTACAAGCTGTC |
| | | CCAGTTCACCTTCAAAAAGCTGAGCGACCT |
| | | GGTGGACTACAAGGCCAAGCGCGAGGGCA |
| | | TCAAAGTGATCTACGTGGAACCCGCCTACA |
| | | CCAGCAAGGACTGTTCTCACTGTGGCGAGA |
| | | GAGTGAACACCCAGCGGCCTTTCAACGGCA |
| | | ACTTCAGCCTGTTCAAGTGCAACAAGTGCG |
| | | GCATCGTGCTGAACAGCGACTACAACGCCA |
| | | GCCTGAATATCGCCCGGAAGGGCCTGAAC |
| | | ATCAGCGCCAATTGA |
| Super-coiled | SEQ ID NO: 96 | TATTAAATACTCGTATTGCTGTTCGATT |
| plasmid DNA | | AT |
| target sequence | | |
| Cas14a.3 crRNA | SEQ ID NO: 97 | GUUGCAGAACCCGAAUAGACGAAUGA |
| | | AGGAAUGCAACUAUUAAAUACUCGUA |
| | | UUGCUGU |
| Cas14a.3 tracr3 | SEQ ID NO: 98 | CGAUUCCUCCCUACAGUAGUUAGGUA |
| | | UAGCCGAAAGGUAGAGACUAAAUCUG |
| | | UAGUUGGAGUGGGCCGCUUGCAUCGG |
| | | CCUAAAGUUGAGAAGUGUCAGACUCU |
| | | GAUAACCCUCAACGACGAUAUUCUUU |
| | | AUUUC |
| Cas14a.3 tracr4 | SEQ ID NO: 99 | CGAUUCCUCCCUACAGUAGUUAGGUA |
| | | UAGCCGAAAGGUAGAGACUAAAUCUG |
| | | UAGUUGGAGUGGGCCGCUUGCAUCGG |
| | | CCUAAAGUUGAGAAGUGUCAGACUCU |
| | | GAUAACCCUCAACGACGAUAUUCUUU |
| | | AUUUCGGUUCAAAGUUCUGCACAAAA |
| | | CAGGUGAGUCCUUAUAAACCGGUG |
| Cas14b.3 crRNA | SEQ ID NO: 100 | CUUUCAUACUCAGAACAAAGGGAUUA |
| | | AGGAAUGCAACUAUUAAAUACUCGUA |
| | | UUGCUGU |
| Cas14b.3 tracr 2 | SEQ ID NO: 101 | GAACAGACCAAUCUUUAAUUCCGUUC |
| | | UGAUUUAAAAAAUCAGAAUCUCUUUA |
| | | UAAAUAGUAUUACAAAAAGUGUACAU |
| | | UCCAAAAUCCGAAAGCAGAAUUGACC |
| | | UUUUUAAG |
| Cas14b.3 tracr 3 | SEQ ID NO: 151 | AUGCGGAAGAUUUGGCGUUGUUGUAA |
| | | CGCAAUAAGGGGUAACCCUGAAAAGG |
| | | UUUGAAAUCAUAUAAACCUAGUUUUA |
| | | UUUGAGUUUAGGCUCAGAUAAAAUGA |
| | | ACAGACCAAUCUUUAAUUCCGUUCUG |
| | | AUUUAAAAAAUCAGAAUCUCUUUAUA |
| | | AAUAGUAUUACAAAAAGUGUACAUUC |
| | | CAAAAUCCGAAAGCAGAAUUGACC |
| Cas14b.4 crRNA | SEQ ID NO: 102 | AUUUCAUACUCAGAACAAAGGGAUUA |
| | | AGGAAUGCAACUAUUAAAUACUCGUA |
| | | UUGCUGU |
| Cas14b.4 tracr1 | SEQ ID NO: 103 | UUGGUUAAGCCAAGAUAUGGAAUGCC |
| | | AUUGUAAUAUUAUGGUGUUGACUUAG |
| | | UUUAGAUUUAAACAAUCUUCGAUGGC |
| | | UAUAUGCGGAAGGUUUGGCGUCGUUG |
| | | UAACGCAAUAGGGGGCGACCCCGAAA |
| | | AGGUUUGAUAUCAUAUCAAACUUAGU |
| | | UUUGUUUAAGUUAAGGCUUAUUCAAA |
| | | AUGAACAGACCAAUUCUUAAUACCUU |
| | | UAUCUGACU |
| Cas14b.10 | SEQ ID NO: 104 | GUUGCGCGAAUAGAAUAAAGGAAUUA |
| crRNA | | AGGAAUGCAACUAUUAAAUACUCGUA |
| | | UUGCUGU |
| Cas14b.10 tracr1 | SEQ ID NO: 105 | AGUGUAAGUUGAAGUGUGAGCUUAUG |
| | | GAUUAUUAUUUACAAAAUAAUACUGA |
| | | CUUACUAAGAUAUCUUGAGGGUAUAC |
| | | CCAAAAAGAUUGGCGUUGUUGCAACG |
| | | CAAUAAGAUGUAAAUCUGAAAAGGUU |
| | | UGAAAUCAUAUAAGUAAUUUUAUUUG |
| | | AGUUUC GGCUUGAGUAAAAUGAACAG |
| | | ACCAAUUUUUAAUUUCGUUCAUAUCA |
| | | UCGCAACUA |

Cas14-guide RNA ribonucleoprotein complexes were incubated for 60 minutes at 37° C. in Tris, pH 7.9 buffer (50 mM potassium acetate, 20 mM tris-acetate, 10 mM magnesium acetate, 100 μg/ml BSA) in the presence of super-coiled plasmid DNA containing the target sequence of SEQ ID NO: 96 immediately 3′ of TTTA PAM sites. The sequence of the super-coiled plasmid DNA is

GTGTAGATAACTACGATACGGGAGGGCTTACCATCTGGCCCCAGTGCTGC
AATGATACCGCGAGACCCACGCTCACCGGCTCCAGATTTATCAGCAATAA
ACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCAC
TCATGGTTATGGCAGCACTGCATAATTCTCTTACTGTCATGCCATCCGTA
AGATGCTTTTCTGTGACTGGTGAGTACTCAACCAAGTCATTCTGAGAATA
GTGTATGCGGCGACCGAGTTGCTCTTGCCCGGCGTCAATACGGGATAATA
CCGCGCCACATAGCAGAACTTTAAAAGTGCTCATCATTGGAAAACGTTCT
TCGGGGCGAAAACTCTCAAGGATCTTACCGCTGTTGAGATCCAGTTCGAT
GTAACCCACTCGTGCACCCAACTGATCTTCAGCATCTTTTACTTTCACCA
GCGTTTCTGGGTGAGCAAAAACAGGAAGGCAAAATGCCGCAAAAAAGGGA
ATAAGGGCGACACGGAAATGTTGAATACTCATACTCTTCCTTTTTCAATA
TTATTGAAGCATTTATCAGGGTTATTGTCTCATGAGCGGATACATATTTG
AATGTATTTAGAAAAATAAACAAATAGGGGTTCCGCGCACATTTCCCCGA
AAAGTGCCACCTGACGTCTAAGAAACCATTATTATCATGACATTAACCTA
TAAAAATAGGCGTATCACGAGGCCCTTTCGTCTCGCGCGTTTCGGTGATG
ACGGTGAAAACCTCTGACACATGCAGCTCCCGGAGACGGTCACAGCTTGT
CTGCCATGGACATGTTTATATTAAATACTCGTATTGCTGTTCGATTATGA
CCGAATTCCCTGTCGTGCCAGCTGCATTAATGAATCGGCCAACGCGCGGG
GAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAG
GCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGA
CGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTAT
CCGGTAACTATCGTCTTGAGTCCAACCCGGTAAGACACGACTTATCGCCA
CTGGCAGCAGCCACTGGTAACAGGATTAGCAGAGCGAGGTATGTAGGCGG
TGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCA
CGTTAAGGGATTTTGGTCATGAGATTATCAAAAAGGATCTTCACCTAGAT
CCTTTTAAATTAAAAATGAAGTTTTAAATCAATCTAAAGTATATATGAGT
AAACTTGGTCTGACAGTTACCAATGCTTAATCAGTGAGGCACCTATCTCA
GCGATCTGTCTATTTCGTTCATCCATAGTTGCCTGACTCCCCGTC
(SEQ ID NO: 152; target sequence is bolded and
underlined).

During the 60-minute incubations, the ribonucleoproteins performed nicking and cleavage on the plasmids. After 60 minutes, the reactions were quenched with 1 mg/ml proteinase K, 0.08% SDS and 15 mM EDTA.

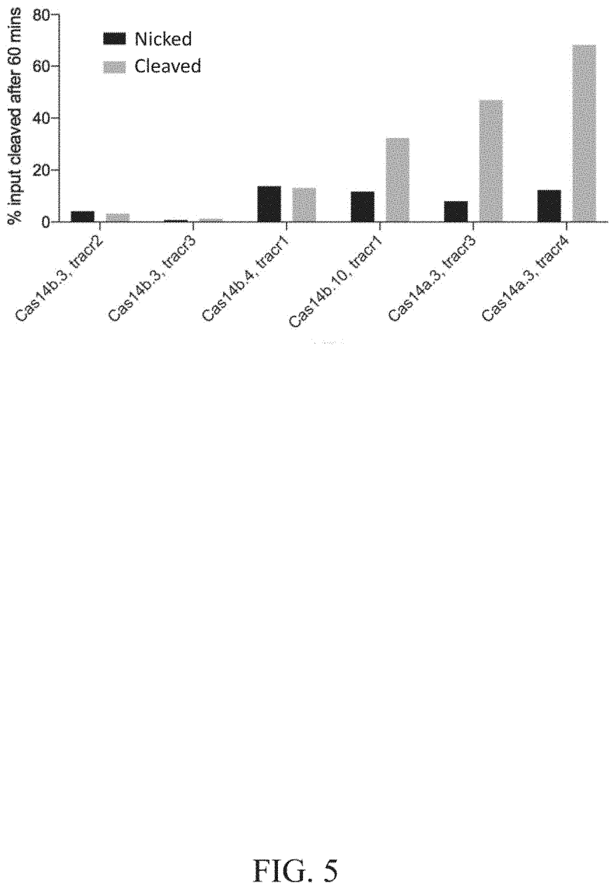

The plasmids were then analyzed for nicking and cleavage by gel-electrophoresis on agarose gel. The percentage of plasmids that underwent cis- and trans-cleavage in each assay are shown in FIG. 5. The assays show that nicking and cleavage activity vary between Cas14a and Cas14b orthologs, and that programmable nickase nicking and cleavage activity are dependent on tracrRNA sequence.

### Example 8

### Dependence of Cas14a and Cas14b Nicking and Cleavage Activity on tracrRNA Sequence

This example shows the dependence of the nicking cleavage activity of different programmable nickases on tracrRNA sequence. SEQ ID NO: 1, SEQ ID NO: 11, and SEQ ID NO: 17 were separately complexed with eighteen distinct guide RNAs. The guide RNAs contained identical spacer sequences targeting SEQ ID NO: 96 and distinct tracr sequences. The sequences of the programmable nickases and guide RNAs used in the assays are summarized in TABLE 4.

TABLE 4
Guide Nucleic Acid, Target Nucleic Acid and Programmable Nickase
Sequences

| Species | SEQ ID NO | Sequence |
|---|---|---|
| Cas14a.3 | SEQ ID NO: 1 | MEVQKTVMKTLSLRILRPLYSQEIEKEIKE |
| Programmable | | EKERRKQAGGTGELDGGFYKKLEKKHSE |
| Nickase | | MFSFDRLNLLLNQLQREIAKVYNHAISEL |
| | | YIATIAQGNKSNKHYISSIVYNRAYGYFYN |
| | | AYIALGICSKVEANFRSNELLTQQSALPTA |
| | | KSDNFPIVLHKQKGAEGEDGGFRISTEGSD |
| | | LIFEIPIPFYEYNGENRKEPYKWVKKGGQK |
| | | PVLKLILSTFRRQRNKGWAKDEGTDAEIR |
| | | KVTEGKYQVSQIEINRGKKLGEHQKWFA |
| | | NFSIEQPIYERKPNRSIVGGLDVGIRSPLVC |
| | | AINNSFSRYSVDSNDVFKFSKQVFAFRRRL |
| | | LSKNSLKRKGHGAAHKLEPITEMTEKNDK |
| | | FRKKIIERWAKEVTNFFVKNQVGIVQIEDL |
| | | STMKDREDHFFNQYLRGFWPYYQMQTLI |
| | | ENKLKEYGIEVKRVQAKYTSQLCSNPNCR |
| | | YWNNYFNFEYRKVNKFPKFKCEKCNLEIS |
| | | ADYNAARNLSTPDIEKFVAKATKGINLPE |
| | | K |
| Cas14.b4 | SEQ ID NO: 11 | MPKQDLVTTGIKFKLDVDKETRKKLDDY |
| Programmable | | FDEYGKAINFAVKIIQKNLKEDRFAGKIAL |
| Nickase | | GEDKKPLLDKDGKKIYNYPNESCSCGNQV |
| | | RRYVNAKPFCVDCYKLKFTENGIRKRMY |
| | | SARGRKADSDINIKNSTNKISKTHFNYAIR |
| | | EGFILDKSLKKQRSKRIKKLLELKRKLQEF |
| | | IDIRQGQMVLCPKIKNQRVDKFIHPSWLK |
| | | RDKKLEEFRGYSLSVVEGKIKIFNRNILRE |
| | | EDSLRQRGHVNFKANRIMLDKSVRFLDG |
| | | GKVNFNLNKGLPKEYLLDLPKKENKLSW |
| | | LNEKISLIKLQKPKYAYLLRREGSFFIQYTI |
| | | ENVPKTFSDYLGAIGIDRGISHIAVCTFVSK |
| | | NGVNKAPVFFSSGEILKLKSLQKQRDLFLR |
| | | GKHNKIRKKSNMRNIDNKINLILHKYSRNI |
| | | VNLAKSEKAFIVFEKLEKIKKSRFKMSKSL |
| | | QYKLSQFTFKKLSDLVEYKAKIEGIKVDY |
| | | VPPEYTSKECSHCGEKVDTQRPFNGNSSLF |
| | | KCNKCRVQLNADYNASINIAKKSLNISN |
| Cas14.b10 | SEQ ID NO: 17 | MEKNNSEQTSITTGIKFKLKLDKETKEKLN |
| Programmable | | NYFDEYGKAINFAVRIIQMQLNDDRLAGK |
| Nickase | | YKRDEKGKPILGEDGKKILEIPNDFCSCGN |
| | | QVNHYVNGVSFCQECYKKRFSENGIRKR |
| | | MYSAKGRKAEQDINIKNSTNKISKTHFNY |
| | | AIREAFNLDKSIKKQREKRFKKLKDMKRK |
| | | LQEFLEIRDGKRVICPKIEKQKVERYIHPS |
| | | WINKEKKLEEFRGYSLSIVNSKIKSFDRNI |
| | | QREEKSLKEKGQINFKAQRLMLDKSVKFL |
| | | KDNKVSFTISKELPKTFELDLPKKEKKLN |
| | | WLNEKLEIIKNQKPKYAYLLRKENNIFLQ |
| | | YTLDSIPEIHSEYSGAVGIDRGVSHIAVYTF |
| | | LDKDGKNERPFFLSSSGILRLKNLQKERDK |
| | | FLRKKHNKIRKKGNMRNIEQKINLILHEYS |
| | | KQIVNFAKDKNAFIVFELLEKPKKSRERM |
| | | SKKIQYKLSQFTFKKLSDLVDYKAKREGI |
| | | KVIYVEPAYTSKDCSHCGERVNTQRPFNG |
| | | NFSLFKCNKCGIVLNSDYNASLNIARKGL |
| | | NISAN |
| Super-coiled | SEQ ID NO: 96 | TATTAAATACTCGTATTGCTGTTCGA |
| Plasmid DNA | | TTAT |
| target sequence | | |
| Cas14a.3 crRNA | SEQ ID NO: 97 | GUUGCAGAACCCGAAUAGACGAAU |
| | | GAAGGAAUGCAACUAUUAAAUACU |
| | | CGUAUUGCUGU |
| Cas14a.3 tracr1 | SEQ ID NO: 106 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGU |
| Cas14a.3 tracr2 | SEQ ID NO: 107 | CGAUUCCUCCCUACAGUAGUUAGGU |
| | | AUAGCCGAAAGGUAGAGACUAAAU |
| | | C |
| Cas14a.3 tracr3 | SEQ ID NO: 98 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUC |
| Cas14a.3 tracr4 | SEQ ID NO: 99 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUCGGUUCAAAGUU |
| | | CUGCACAAAACAGGUGAGUCCUUAU |
| | | AAACCGGUG |
| Cas14a.3 tracr5 | SEQ ID NO: 108 | GUUGGAGUGGGCCGCUUGCAUCGGC |
| | | CUAAAGUUGAGAAGUGUCAGACUC |
| | | UGAUAACCCUCAACGACGAUAUUCU |
| | | UUAUUUCGGUUC |
| Cas14a.3 tracr6 | SEQ ID NO: 109 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| Cas14a.3 tracr7 | SEQ ID NO: 110 | CAUCGGCCUAAAGUUGAGAAGUGU |
| | | CAGACUCUGAUAACCCUCAACGACG |
| | | AUAUUCUUUAUUUCGGUUCAAAGU |
| | | UCUGCACAAAACAGGUGAGUCCUUA |
| | | UAAACCGGUGUGCAGAACGCCGGCU |
| | | CACCUUUUUCCUUCAUC |
| Cas14a.3 tracr8 | SEQ ID NO: 111 | UAGGUAUAGCCGAAAGGUAGAGAC |
| | | UAAAUCUGUAGUUGGAGUGGGCCG |
| | | CUUGCAUCGGCCUAAAGUUGAGAA |
| | | GUGUCAGACUCUGAUAACCCUCAAC |
| | | GACGAUAUUCUUUAUUUC |
| Cas14a.3 tracr9 | SEQ ID NO: 112 | CUACAGUAGUUAGGUAUAGCCGAA |
| | | AGGUAGAGACUAAAUCUGUAGUUG |
| | | GAGUGGGCCGCUUGCAUCGGCCUAA |
| | | AGUUGAGAAGUGUCAGACUCUGAU |
| | | AACCCUCAACGACGAUAUUCUUUAU |
| | | UUC |
| Cas14a.3 tracr10 | SEQ ID NO: 113 | CUCCCUACAGUAGUUAGGUAUAGCC |
| | | GAAAGGUAGAGACUAAAUCUGUAG |
| | | UUGGAGUGGGCCGCUUGCAUCGGCC |
| | | UAAAGUUGAGAAGUGUCAGACUCU |
| | | GAUAACCCUCAACGACGAUAUUCUU |
| | | UAUUUC |
| Cas14a.3 tracr11 | SEQ ID NO: 114 | GCACACGAUUCCUCCCUACAGUAGU |
| | | UAGGUAUAGCCGAAAGGUAGAGAC |
| | | UAAAUCUGUAGUUGGAGUGGGCCG |
| | | CUUGCAUCGGCCUAAAGUUGAGAA |
| | | GUGUCAGACUCUGAUAACCCUCAAC |
| | | GACGAUAUUCUUUAUUUC |
| Cas14a.3 tracr12 | SEQ ID NO: 115 | UUGAUGCACACGAUUCCUCCCUACA |
| | | GUAGUUAGGUAUAGCCGAAAGGUA |
| | | GAGACUAAAUCUGUAGUUGGAGUG |
| | | GGCCGCUUGCAUCGGCCUAAAGUUG |
| | | AGAAGUGUCAGACUCUGAUAACCCU |
| | | CAACGACGAUAUUCUUUAUUUC |
| Cas14a.3 tracr13 | SEQ ID NO: 116 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUU |
| Cas14a.3 tracr14 | SEQ ID NO: 117 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUCGGUUC |
| Cas14a.3 tracr15 | SEQ ID NO: 118 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUCGGUUCAAAGUU |
| | | CUGCACAAA |
| Cas14a.3 tracr16 | SEQ ID NO: 119 | GUUGGAGUGGGCCGCUUGCAUCGGC |
| | | CUAAAGUUGAGAAGUGUCAGACUC |
| | | UGAUAACCCUCAACGACGAUAUUCU |
| | | UUAUUUC |
| Cas14a.3 tracr17 | SEQ ID NO: 120 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUCGGUUCAAAGUU |
| | | CUGCACAAAACAGGUGAGUCCUUA |
| Cas14a.3 tracr18 | SEQ ID NO: 121 | CGAUUCCUCCCUACAGUAGUUAGGU |
| | | AUAGCCGAAAGGUAGAGACUAAAU |
| | | CUGUAGUUGGAGUGGGCCGCUUGC |
| | | AUCGGCCUAAAGUUGAGAAGUGUC |
| | | AGACUCUGAUAACCCUCAACGACGA |
| | | UAUUCUUUAUUUCGGUUCAAAGUU |
| | | CUGCACAAAACAGGUGAGUCCUUAU |
| | | AAACCGGUGUGCAGAACG |
| Cas14b.4 crRNA | SEQ ID NO: 102 | AUUUCAUACUCAGAACAAAGGGAU |
| | | UAAGGAAUGCAACUAUUAAAUACU |
| | | CGUAUUGCUGU |
| Cas14b.4 tracr 1 | SEQ ID NO: 103 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCUUUAUCUGACU |
| Cas14b.4 tracr 2 | SEQ ID NO: 122 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AA |
| Cas14b.4 tracr 3 | SEQ ID NO: 123 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAAC |
| Cas14b.4 tracr 4 | SEQ ID NO: 124 | AUUGUAAUAUUAUGGUGUUGACUU |
| | | AGUUUAGAUUUAAACAAUCUUCGA |
| | | UGGCUAUAUGCGGAAGGUUUGGCG |
| | | UCGUUGUAACGCAAUAGGGGGCGA |
| | | CCCCGAAAAGGUUUGAUAUCAUAUC |
| | | AAAC |
| Cas14b.4 tracr 5 | SEQ ID NO: 125 | AGCUCUUUUGUUGGUUAAGCCAAG |
| | | AUAUGGAAUGCCAUUGUAAUAUUA |
| | | UGGUGUUGACUUAGUUUAGAUUUA |
| | | AACAAUCUUCGAUGGCUAUAUGCG |
| | | GAAGGUUUGGCGUCGUUGUAACGC |
| | | AAUAGGGGGCGACCCCGAAAAGGU |
| | | UUGAUAUCAUAUCAAACUUAGUUU |
| | | UGUUUAAGUUAAGGCUUAUUCAAA |
| | | AUGAACAGACCAAUUCUUAAUACCU |
| | | UUAUCUGACU |
| Cas14b.4 tracr 6 | SEQ ID NO: 126 | UCCAAUAACUAGCUCUUUUGUUGG |
| | | UUAAGCCAAGAUAUGGAAUGCCAU |
| | | UGUAAUAUUAUGGUGUUGACUUAG |
| | | UUUAGAUUUAAACAAUCUUCGAUG |
| | | GCUAUAUGCGGAAGGUUUGGCGUC |
| | | GUUGUAACGCAAUAGGGGGCGACCC |
| | | CGAAAAGGUUUGAUAUCAUAUCAA |
| | | ACUUAGUUUUGUUUAAGUUAAGGC |
| | | UUAUUCAAAAUGAACAGACCAAUU |
| | | CUUAAUACCUUUAUCUGACU |
| Cas14b.4 tracr 7 | SEQ ID NO: 127 | CAAGAUAUGGAAUGCCAUUGUAAU |
| | | AUUAUGGUGUUGACUUAGUUUAGA |
| | | UUUAAACAAUCUUCGAUGGCUAUA |
| | | UGCGGAAGGUUUGGCGUCGUUGUA |
| | | ACGCAAUAGGGGGCGACCCCGAAAA |
| | | GGUUUGAUAUCAUAUCAAACUUAG |
| | | UUUUGUUUAAGUUAAGGCUUAUUC |
| | | AAAAUGAACAGACCAAUUCUUAAU |
| | | ACCUUUAUCUGACU |
| Cas14b.4 tracr 8 | SEQ ID NO: 128 | AAUGCCAUUGUAAUAUUAUGGUGU |
| | | UGACUUAGUUUAGAUUUAAACAAU |
| | | CUUCGAUGGCUAUAUGCGGAAGGU |
| | | UUGGCGUCGUUGUAACGCAAUAGG |
| | | GGGCGACCCCGAAAAGGUUUGAUA |
| | | UCAUAUCAAACUUAGUUUUGUUUA |
| | | AGUUAAGGCUUAUUCAAAAUGAAC |
| | | AGACCAAUUCUUAAUACCUUUAUCU |
| | | GACU |
| Cas14b.4 tracr 9 | SEQ ID NO: 129 | UAAUAUUAUGGUGUUGACUUAGUU |
| | | UAGAUUUAAACAAUCUUCGAUGGC |
| | | UAUAUGCGGAAGGUUUGGCGUCGU |
| | | UGUAACGCAAUAGGGGGCGACCCCG |
| | | AAAAGGUUUGAUAUCAUAUCAAAC |
| | | UUAGUUUUGUUUAAGUUAAGGCUU |
| | | AUUCAAAAUGAACAGACCAAUUCU |
| | | UAAUACCUUUAUCUGACU |
| Cas14b.4 tracr 10 | SEQ ID NO: 130 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCUUUAUCUGACU |
| | | AAACGUCAGA |
| Cas14b.4 tracr 11 | SEQ ID NO: 131 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCUUUAUCUGACU |
| | | AAACGUCAGAACAUCUUUAU |
| Cas14b.4 tracr 12 | SEQ ID NO: 132 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCUUUAUCUGACU |
| | | AAACGUCAGAACAUCUUUAUAAAU |
| | | CAAUUU |
| Cas14b.4 tracr 13 | SEQ ID NO: 133 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCUUUAUCUGACU |
| | | AAACGUCAGAACAUCUUUAUAAAU |
| | | CAAUUUACAAAAAUGA |
| Cas14b.4 tracr 14 | SEQ ID NO: 134 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUUCUUAAUACCU |
| Cas14b.4 tracr 15 | SEQ ID NO: 135 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAAAUGAACAGAC |
| | | CAAUU |
| Cas14b.4 tracr 16 | SEQ ID NO: 136 | UUGGUUAAGCCAAGAUAUGGAAUG |
| | | CCAUUGUAAUAUUAUGGUGUUGAC |
| | | UUAGUUUAGAUUUAAACAAUCUUC |
| | | GAUGGCUAUAUGCGGAAGGUUUGG |
| | | CGUCGUUGUAACGCAAUAGGGGGC |
| | | GACCCCGAAAAGGUUUGAUAUCAU |
| | | AUCAAACUUAGUUUUGUUUAAGUU |
| | | AAGGCUUAUUCAAA |
| Cas14b.10 crRNA | SEQ ID NO: 104 | GUUGCGCGAAUAGAAUAAAGGAAU |
| | | UAAGGAAUGCAACUAUUAAAUACU |
| | | CGUAUUGCUGU |
| Cas14b.10 tracr 1 | SEQ ID NO: 105 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUCAUCGCAACUA |
| Cas14b.10 tracr 2 | SEQ ID NO: 137 | AUUUUAUUUGAGUUUCGGCUUGAG |
| | | UAAAAUGAACAGACCAAUUUUUAA |
| | | UUUCGUUCAUAUCAUCGCAACUAAG |
| | | AAUUCCUUUAAAAAGGAAAUGCAG |
| | | AAAAUGGACAUUCCAAAAUUCAAA |
| | | ACAAAAUUCGGUUUUUUAAGACCA |
| | | GAAAG |
| Cas14b.10 tracr 3 | SEQ ID NO: 138 | ACUGACUUACUAAGAUAUCUUGAG |
| | | GGUAUACCCAAAAAGAUUGGCGUU |
| | | GUUGCAACGCAAUAAGAUGUAAAU |
| | | CUGAAAAGGUUUGAAAUCAUAUAA |
| | | GUAAUUUUAUUUGAGUUUCGGCUU |
| | | GAGUAAAAUGAACAGACCAAUUUU |
| | | UAAUUUCGUUCAUAUCAUCGCAACU |
| | | A |
| Cas14b.10 tracr 4 | SEQ ID NO: 139 | GAAGUGUGAGCUUAUGGAUUAUUA |
| | | UUUACAAAAUAAUACUGACUUACU |
| | | AAGAUAUCUUGAGGGUAUACCCAA |
| | | AAAGAUUGGCGUUGUUGCAACGCA |
| | | AUAAGAUGUAAAUCUGAAAAGGUU |
| | | UGAAAUCAUAUAAGUAAUUUUAUU |
| | | UGAGUUUCGGCUUGAGUAAAAUGA |
| | | ACAGACCAAUUUUUAAUUUCGUUC |
| | | AUAUCAUCGCAACUA |
| Cas14b.10 tracr 5 | SEQ ID NO: 140 | CUUAUGGAUUAUUAUUUACAAAAU |
| | | AAUACUGACUUACUAAGAUAUCUU |
| | | GAGGGUAUACCCAAAAAGAUUGGC |
| | | GUUGUUGCAACGCAAUAAGAUGUA |
| | | AAUCUGAAAAGGUUUGAAAUCAUA |
| | | UAAGUAAUUUUAUUUGAGUUUCGG |
| | | CUUGAGUAAAAUGAACAGACCAAU |
| | | UUUUAAUUUCGUUCAUAUCAUCGC |
| | | AACUA |
| Cas14b.10 tracr 6 | SEQ ID NO: 141 | AUUAUUUACAAAAUAAUACUGACU |
| | | UACUAAGAUAUCUUGAGGGUAUAC |
| | | CCAAAAAGAUUGGCGUUGUUGCAA |
| | | CGCAAUAAGAUGUAAAUCUGAAAA |
| | | GGUUUGAAAUCAUAUAAGUAAUUU |
| | | UAUUUGAGUUUCGGCUUGAGUAAA |
| | | AUGAACAGACCAAUUUUUAAUUUC |
| | | GUUCAUAUCAUCGCAACUA |
| Cas14b.10 tracr 7 | SEQ ID NO: 142 | AAAUAAUACUGACUUACUAAGAUA |
| | | UCUUGAGGGUAUACCCAAAAAGAU |
| | | UGGCGUUGUUGCAACGCAAUAAGA |
| | | UGUAAAUCUGAAAAGGUUUGAAAU |
| | | CAUAUAAGUAAUUUUAUUUGAGUU |
| | | UCGGCUUGAGUAAAAUGAACAGAC |
| | | CAAUUUUUAAUUUCGUUCAUAUCA |
| | | UCGCAACUA |
| Cas14b.10 tracr 8 | SEQ ID NO: 143 | UGAACAUCAGCGCCAAUUGAAGUG |
| | | UAAGUUGAAGUGUGAGCUUAUGGA |
| | | UUAUUAUUUACAAAAUAAUACUGA |
| | | CUUACUAAGAUAUCUUGAGGGUAU |
| | | ACCCAAAAAGAUUGGCGUUGUUGC |
| | | AACGCAAUAAGAUGUAAAUCUGAA |
| | | AAGGUUUGAAAUCAUAUAAGUAAU |
| | | UUUAUUUGAGUUUCGGCUUGAGUA |
| | | AAAUGAACAGACCAAUUUUUAAUU |
| | | UCGUUCAUAUCAUCGCAACUA |
| Cas14b.10 tracr 9 | SEQ ID NO: 144 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUCAUCGCAACUA |
| | | AGAAUUCCUU |
| Cas14b.10 tracr 10 | SEQ ID NO: 145 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUCAUCGCAACUA |
| | | AGAAUUCCUUUAAAAAGGAA |
| Cas14b.10 tracr 11 | SEQ ID NO: 146 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUCAUCGCAACUA |
| | | AGAAUUCCUUUAAAAAGGAAAUGC |
| | | AGAAAA |
| Cas14b.10 tracr 12 | SEQ ID NO: 147 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUCAUCGCAACUA |
| | | AGAAUUCCUUUAAAAAGGAAAUGC |
| | | AGAAAAUGGACAUUCCAAAAUUCA |
| | | AAACAAAAUUCGGUUUUUUAAGAC |
| | | CAGAAAG |
| Cas14b.10 tracr 13 | SEQ ID NO: 148 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUUCGUUCAUAUC |
| Cas14b.10 tracr 14 | SEQ ID NO: 149 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAAUUUUU |
| | | AAUUU |
| Cas14b.10 tracr 15 | SEQ ID NO: 150 | AGUGUAAGUUGAAGUGUGAGCUUA |
| | | UGGAUUAUUAUUUACAAAAUAAUA |
| | | CUGACUUACUAAGAUAUCUUGAGG |
| | | GUAUACCCAAAAAGAUUGGCGUUG |
| | | UUGCAACGCAAUAAGAUGUAAAUC |
| | | UGAAAAGGUUUGAAAUCAUAUAAG |
| | | UAAUUUUAUUUGAGUUUCGGCUUG |
| | | AGUAAAAUGAACAGACCAA |

The Cas14-guide RNA complexes were then incubated at 37° C. in Tris, pH 7.9 buffer (50 mM potassium acetate, 20 mM tris-acetate, 10 mM magnesium acetate, 100 pg/ml BSA) in the presence of super-coiled plasmid DNA containing the target sequence of SEQ ID NO: 96 immediately 3′ of TTTA PAM sites. The sequence of the super-coiled plasmid DNA is (SEQ ID NO: 152; target sequence is shown in bold and underlining). The programmable nickases performed nicking and cleavage on the plasmids during the period. After 10 minutes, the reactions were quenched with 1 mg/ml proteinase K, 0.08% SDS and 15 mM EDTA.

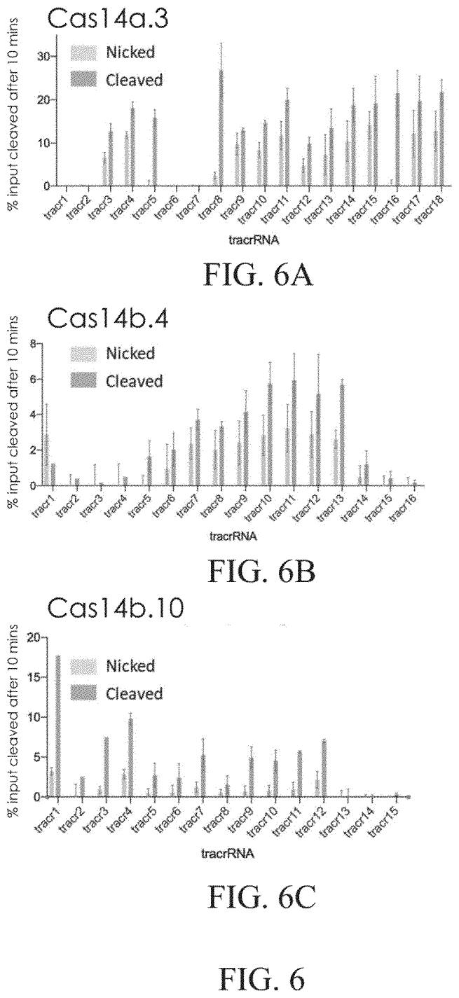

The plasmids were then analyzed for nicking and cleavage by gel-electrophoresis on agarose gel. The percentage of plasmids that were nicked and the percentage of plasmids that were cleaved in each assay are shown in FIG. 6. The results for Cas14a.3 are shown in FIG. 6A. The results for Cas14b.4 are shown in FIG. 6B. The results for Cas14b.10 are shown in FIG. 6C. The results show that nicking and cleavage activity varies between types of programmable nickases, and that the rates of nicking and cleavage performed by a programmable nickase can be controlled by optimization of the tracr sequence.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of introducing a break in a target nucleic acid, the method comprising introducing the break by contacting the target nucleic acid with:
(a) a first guide nucleic acid comprising a first region that binds to a first programmable nickase having a length of no more than 900 amino acids; and
(b) a second guide nucleic acid comprising a first region that binds to a second programmable nickase having a length of no more than 900 amino acids,
wherein the first guide nucleic acid comprises a second region that binds to the target nucleic acid and wherein the second guide nucleic acid comprises a second region that binds to the target nucleic acid and wherein the second region of the first guide nucleic acid and the second region of the second guide nucleic acid bind opposing strands of the target nucleic acid.

2. The method of claim 1, wherein the first programmable nickase and the second programmable nickase have a length of from 350 to 900 amino acids.

3. The method of any one of claims 1-2, wherein the first programmable nickase and the second programmable nickase have a length of from 480 to 550 amino acids.

4. The method of any one of claims 1-3, wherein the first programmable nickase and second programmable nickase are a Type V CRISPR/Cas enzyme.

5. The method of claim 4, wherein the Type V CRISPR/Cas enzyme comprises three partial RuvC domains.

6. The method of claim 5, wherein the three partial RuvC domains are RuvC-I, RuvC-II, and RuvC-III subdomains.

7. The method of any one of claims 1-6, wherein the first programmable nickase and the second programmable nickase are a Cas14 protein.

8. The method of claim 7, wherein the Cas14 protein is a Cas14a protein, a Cas14b protein, a Cas14c protein, a Cas14d protein, or a Cas14e protein.

9. The method of any one of claims 7-8, wherein the Cas14 protein is a Cas14a protein.

10. The method of any one of claims 7-8, wherein the Cas14 proteins is a Cas14b protein.

11. The method of any one of claims 7-8, wherein the Cas14 protein is a Cas14e protein.

12. The method of any one of claims 1-11, wherein the first programmable nickase, the second programmable nickase, or both have at least 80%, at least 85%, at least 90%, at least 92%, at least 95%, at least 97%, or at least 99% sequence identity with any one of SEQ ID NO: 1-SEQ ID NO: 91 or SEQ ID NO: 170.

13. The method of any one of claims 1-12, wherein the first programmable nickase, the second programmable nickase, or both are any one of SEQ ID NO: 1-SEQ ID NO: 91 or SEQ ID NO: 170.

14. The method of any one of claims 1-13, wherein the first programmable nickase, the second programmable nickase, or both have at least 80%, at least 85%, at least 90%, at least 92%, at least 95%, at least 97%, or at least 99% sequence identity with SEQ ID NO: 1.

15. The method of any one of claims 1-14, wherein the first programmable nickase, the second programmable nickase, or both are SEQ ID NO: 1.

16. The method of any one of claims 1-13, wherein the first programmable nickase, the second programmable nickase, or both have at least 80%, at least 85%, at least 90%, at least 92%, at least 95%, at least 97%, or at least 99% sequence identity with SEQ ID NO: 10.

17. The method of any one of claim 1-13 or 16, wherein the first programmable nickase, the second programmable nickase, or both are SEQ ID NO: 10.

18. The method of any one of claims 1-13, wherein the first programmable nickase, the second programmable nickase, or both have at least 80%, at least 85%, at least 90%, at least 92%, at least 95%, at least 97%, or at least 99% sequence identity with SEQ ID NO: 11.

19. The method of any one of claim 1-13 or 18, wherein the first programmable nickase, the second programmable nickase, or both are SEQ ID NO: 11.

20. The method of any one of claims 1-13, wherein the first programmable nickase, the second programmable nickase, or both have at least 80%, at least 85%, at least 90%, at least 92%, at least 95%, at least 97%, or at least 99% sequence identity with SEQ ID NO: 17.

21. The method of any one of claim 1-13 or 20, wherein the first programmable nickase, the second programmable nickase, or both are SEQ ID NO: 17.

22. The method of any one of claims 1-13, wherein the first programmable nickase, the second programmable nickase, or both have at least 80%, at least 85%, at least 90%, at least 92%, at least 95%, at least 97%, or at least 99% sequence identity with SEQ ID NO: 33.

23. The method of any one of claim 1-13 or 22, wherein the first programmable nickase, the second programmable nickase, or both are SEQ ID NO: 33.

24. The method of any one of claims 1-23, wherein the first guide nucleic acid is a first guide RNA.

25. The method of any one of claims 1-24, wherein the second guide nucleic acid is a second guide RNA.

26. The method of any one of claims 1-25, wherein the first region is a repeat sequence and wherein the second region is a spacer sequence.

27. The method of any one of claims 1-26, wherein the first guide nucleic acid and the second guide nucleic acid comprise a crRNA and a tracrRNA.

28. The method of any one of claims 1-26, wherein the first guide nucleic acid and the second guide nucleic acid comprise a crRNA and a trancRNA.

29. The method of any one of claims 27-28, wherein the crRNA comprises the repeat sequence and the spacer sequence.

30. The method of any one of claims 26-29, wherein the repeat sequence hybridizes to a segment of the tracrRNA.

31. The method of any one of claims 27-30, wherein the tracrRNA has at least 80%, at least 85%, at least 90%, at least 92%, at least 95%, at least 97%, or at least 99% sequence identity with any one of SEQ ID NO: 98-SEQ ID NO: 99, SEQ ID NO: 101, SEQ ID NO: 103, SEQ ID NO: 105-SEQ ID NO: 151.

32. The method of any one of claims 27-31, wherein the tracrRNA is any one of SEQ ID NO: 98-SEQ ID NO: 99, SEQ ID NO: 101, SEQ ID NO: 103, SEQ ID NO: 105-SEQ ID NO: 151.

33. The method of any one of claims 27-31, wherein the tracrRNA has at least 80%, at least 85%, at least 90%, at least 92%, at least 95%, at least 97%, or at least 99% sequence identity with SEQ ID NO: 99

34. The method of any one of claim 27-31 or 33, wherein the tracrRNA is SEQ ID NO: 99.

35. The method of any one of claims 27-31, wherein the tracrRNA has at least 80%, at least 85%, at least 90%, at least 92%, at least 95%, at least 97%, or at least 99% sequence identity with SEQ ID NO: 101.

36. The method of any one of claim 27-31 or 35, wherein the tracrRNA is SEQ ID NO: 101.

37. The method of any one of claims 27-31, wherein the tracrRNA has at least 80%, at least 85%, at least 90%, at least 92%, at least 95%, at least 97%, or at least 99% sequence identity with SEQ ID NO: 103.

38. The method of any one of claim 27-31 or 37, wherein the tracrRNA is SEQ ID NO: 103.

39. The method of any one of claims 27-31, wherein the tracrRNA has at least 80%, at least 85%, at least 90%, at least 92%, at least 95%, at least 97%, or at least 99% sequence identity with SEQ ID NO: 119.

40. The method of any one of claim 27-31 or 39, wherein the tracrRNA is SEQ ID NO: 119.

41. The method of any one of claims 1-40, wherein the first programmable nickase and the second programmable nickase exhibit 2-fold greater nicking activity as compared to double stranded cleavage activity.

42. The method of any one of claims 1-41, wherein the first programmable nickase and the second programmable nickase nick the target nucleic acid at two different sites.

43. The method of any one of claims 1-42, wherein the target nucleic acid comprises double stranded DNA.

44. The method of claim 43, wherein the two different sites are on opposing strands of the double stranded DNA.

45. The method of any one of claims 1-44, wherein the target nucleic acid comprises a mutated sequence or a sequence is associated with a disease.

46. The method of claim 45, wherein the disease is cancer.

47. The method of any one of claims 1-46, wherein the method comprises administering the first programmable nickase and the second programmable nickase to a subject in need thereof.

48. The method of claim 45, wherein the mutated sequence is removed after the first programmable nickase and the second programmable nickase nick the target nucleic acid.

49. The method of any one of claims 1-48, wherein the first programmable nickase and the second programmable nickase are the same.

50. The method of any one of claims 1-49, wherein the first programmable nickase and the second programmable nickase are different.
